(19) United States
(12) Patent Application Publication
(10) Pub. No.: US 2026/0224648 A1
(43) Pub. Date: Aug. 6, 2026

(54) VAGINAL MICROBIOTA COMPOSITIONS FOR TREATING RECURRENT PREGNANCY LOSS

(71) Applicant: Freya Biosciences ApS, Copenhagen Ø (DK)

(72) Inventors: Elleke Fenna BOSMA, Copenhagen Ø (DK); Brynjulf MORTENSEN, Virum (DK); Johan E.T. VAN HYLCKAMA VLIEG, Belmont, MA (US); Kevin DELONG, Baltimore, MD (US); Henriette Svarre NIELSEN, Copenhagen Ø (DK); Tine WRONDING, Copenhagen Ø (DK)

(21) Appl. No.: 19/152,713

(22) PCT Filed: Feb. 1, 2024
(86) PCT No.: PCT/IB2024/050951

§ 371 Date: Jul. 31, 2025

Related U.S. Application Data
(60) Provisional application No. 63/483,003, filed on Feb. 2, 2023.

(51) Int. Cl. A61K 35/747; A61K 9/00; A61K 31/616; A61K 31/727; A61K 35/00; A61K 35/24; A61K 35/74; A61P 15/02

(52) U.S. Cl. CPC A61K 35/747; A61K 9/0034; A61K 31/616; A61K 31/727; A61K 35/24; A61K 35/74; A61P 15/02; A61K 2035/11; A61K 2035/115

(57) ABSTRACT

The invention relates to vaginal compositions for treating recurrent pregnancy loss (RPL) and therapeutic methods to do so.

### RELATED APPLICATIONS

This application claims the benefit under 35 U.S.C. § 119(e) of U.S. Provisional Patent Application No. 63/483,003, filed Feb. 2, 2023, the content of which is incorporated by reference herein in its entirety.

### BACKGROUND OF THE INVENTION

Spontaneous pregnancy loss is a surprisingly common occurrence. Recurrent pregnancy loss (RPL) is defined by the American Society of Reproductive Medicine (ASRM) as two or more failed pregnancies, and is one of the most challenging issues in reproductive medicine. Known causes for RPL include uterine problems, autoimmune diseases, hormonal disorders and genetic abnormalities. However, for a majority of recurrent pregnancy loss cases (RPL) the underlying cause for repeated miscarriages remains unknown. Because of the often unknown etiology and lack of evidence-based diagnostic and treatment strategies, treatment of RPL remains one of the most difficult areas in reproductive medicine. In view of the high degree of emotional stress associated with stillbirth or neonatal death, there exists a high need for a treatment method for RPL.

### SUMMARY OF THE INVENTION

The present invention addresses this unmet need by providing therapeutic methods using vaginal compositions or pharmaceutical compositions comprising the same for use in treating recurrent pregnancy loss (RPL), e.g., by preventing a failed pregnancy, and increasing the chance of a live birth.

In one aspect, the invention provides a vaginal composition for use in a method of treating recurrent pregnancy loss (RPL) in a subject in need thereof, wherein the subject exhibits a dysbiotic microbiota in the genitourinary tract, said method comprising administering an effective amount of the composition to the genitourinary tract of the subject. In embodiments, the vaginal composition comprises:
(i) one, two, three or four bacterial species from the genus Lactobacillus which comprise about 80-99.9% of all detectable bacterial species of the vaginal composition;
(ii) less than 5% of Gardnerella spp., Atopobium spp., and Prevotella spp. of all detectable bacterial species of the vaginal composition; and optionally,
(iii) vaginal transudate and/or mucus, optionally wherein the mucus is cervicovaginal mucus.

In embodiments, the one, two, three or four bacterial species from the genus Lactobacillus are selected from Lactobacillus crispatus, Lactobacillus iners, Lactobacillus jensenii, and/or Lactobacillus gasseri.

In one embodiment, the vaginal composition of the invention is administered to the vaginal cavity of the subject.

The administration of the vaginal composition of the invention reduces the severity of dysbiosis of the dysbiotic microbiota in the genitourinary tract of the subject.

The subject or recipient subject is characterized by a dysbiotic microbiota in the genitourinary tract (e.g., a vaginal dysbiosis or a dysbiosis of the cervicovaginal tract) and further has RPL, i.e., the subject has experienced two or more failed pregnancies.

In one embodiment, the administration of the vaginal composition reverts the dysbiotic microbiota in the genitourinary tract to a Lactobacillus-dominated microbiota.

In one embodiment, the administration of the vaginal composition reduces the risk of miscarriage, loss of the fetus during pregnancy.

The subject may be pregnant or not pregnant. As an example, the subject suffering from RPL may not be pregnant at the first administration of a first dose of the vaginal composition of the invention. Alternatively, the subject suffering from RPL may already be pregnant at the first administration of a first dose of the vaginal composition of the invention. In embodiments, only one dose is administered.

In embodiments, the subject suffering from RPL may not be pregnant at the first administration of a first dose of the vaginal composition of the invention. but may be pregnant at the administration of a second third or fourth dose of the vaginal composition of the invention.

In some embodiments, once pregnancy is diagnosed, the administration of the vaginal composition is maintained for at least 1 month, 2 months or 3 months. In embodiments, the vaginal composition is administered at regular intervals, e.g., weekly, biweekly or monthly, two, three, four or more times. In embodiments, the vaginal composition is administered only once, either in a subject that is pregnant or not pregnant.

In some embodiments, the administration of the vaginal composition promotes and/or results in live birth. Live birth refers to the birth of a viable, live neonate.

In one embodiment, the recurrent pregnancy loss of the subject comprises two or more pregnancy losses. The subject suffering from RPL may have had two failed pregnancies or miscarriages. The subject suffering from RPL may have had three failed pregnancies or miscarriages. The subject suffering from RPL may have had four failed pregnancies or miscarriages. The subject suffering from RPL may have had five failed pregnancies or miscarriages. The subject suffering from RPL may have had six failed pregnancies or miscarriages.

The vaginal composition can be administered to the subject in combination with assisted reproductive treatment. Such assisted reproductive treatment can comprise intrauterine insemination (IUI) or in vitro fertilization (IVF).

In some embodiments, the vaginal composition for use in treating RPL is administered as part of a combination therapy. The vaginal composition can thus be administered in combination with a second therapeutic agent.

The second therapeutic agent may comprise one or more of an anticoagulant, acetyl-salicylic acid, heparin, progesterone, thyroid hormone replacement, metformin, insulin, or an antibiotic agent. In some embodiments, the anticoagulant is a therapeutic anticoagulant or prophylactic anticoagulant. A therapeutic anticoagulant may be administered to treat or alleviate thrombosis in the subject. A prophylactic anticoagulant may be administered in a subject not suffering from thrombosis or has been already treated successfully from thrombosis. In some embodiments, the anticoagulant comprises acetyl salicylic acid and/or heparin. Acetyl salicylic acid may be administered at a daily dose of about 75 mg to 85 mg, e.g., a daily dose of about 75 mg, about 80 mg, or about 85 mg. Heparin may be administered at a daily dose of about 4000 to 5000 Units, such as 4000, 4500, or 5000 Units.

The second therapeutic agent can be administered at the same time as the vaginal composition.

The second therapeutic agent can also be administered at a different time point than the vaginal composition, e.g., prior to or after administration of the vaginal composition.

In some embodiments, the subject having a dysbiotic microbiota in the genitourinary tract is further characterized by having antiphospholipid syndrome (APS), thrombophilia, coeliac disease, uterine abnormalities, hyperprolactinemia, increased inflammation, thyroid malfunction, polycystic ovary syndrome (PCOS), or aberrant insulin metabolism.

The therapeutic method using the vaginal composition of the invention increases the rate of full term births in a population of subjects having dysbiotic microbiota in the genitourinary tract and RPL, relative to a control population, optionally wherein the population of subjects with RPL comprises at least 10, at least 20 or at least 40 subjects.

The vaginal composition of the invention may be formulated into a dosage form. The dosage form can comprise an effective amount of the vaginal composition in one or more discrete units, wherein the effective amount is predetermined.

In some embodiments, the subject is not treated with an antibiotic prior to administration of the vaginal composition.

The vaginal composition can be administered to the vaginal cavity of the subject.

In some embodiments, prior to administering the vaginal composition, a vaginal wash is performed. In some embodiments, the vaginal wash is subsequently rinsed with saline and/or lactic acid. The vaginal wash can be performed with saline, lactic acid or an antiseptic agent, optionally wherein the antiseptic agent is povidone-iodine or chlorohexidine.

In some embodiments, the subject treated for RPL with the vaginal composition of the invention has been diagnosed with bacterial vaginosis or a vaginal infection and is pre-treated with an antibiotic prior to administration of the vaginal composition. In some embodiments, the subject treated for RPL with the vaginal composition of the invention has not been diagnosed with bacterial vaginosis or a vaginal infection and is not pre-treated with an antibiotic prior to administration of the vaginal composition.

In some embodiments, the subject exhibits a symptomatic vaginal dysbiosis. In some embodiments, the subject exhibits an asymptomatic vaginal dysbiosis. In some embodiments, the subject has endometrial and/or vaginal inflammation. The inflammation may be associated with or characterized by elevated vaginal levels of pro-inflammatory or inflammasome-associated cytokines. Pro-inflammatory or inflammasome-associated cytokines can comprise at least one of IL-1α, IL-1β, IL-2, IL-5, IL-6, IL-8, IL-12, IL-15, IL-17, IL-18, IP-10, MCP-1, TNFα, HIF-1α, COX-2 and VEGF, optionally wherein the pro-inflammatory or inflammasome-associated cytokines comprise at least one of IL-1, IL-1α, IL-1β, IL-6, or IL-18, or any combination thereof.

In some embodiments, the subject has elevated vaginal levels of Th1 cytokines. The vaginal composition for use of claim 34, wherein the Th1 cytokines comprise at least one of IFNγ, IL-12, STAMBP or TNF. The subject can be further characterized by reduced vaginal levels of IGF-1, IL-4, IL-10, TGFβ and/or CCL5.

In some embodiments, after administration of the vaginal composition to the subject: the elevated vaginal levels of pro-inflammatory or inflammasome-associated cytokines in the subject are reduced; the elevated vaginal levels of Th1 cytokines in the subject are reduced; and/or the reduced vaginal levels of IGF-1, IL-4, IL-10, TGFβ and/or CCL5 in the subject are increased.

In some embodiments, the vaginal composition of the invention was obtained from a donor subject, wherein the vaginal composition comprises substantially all detectable bacterial, viral, fungal species, and/or metabolites, that are present in the genitourinary tract, e.g., the vagina, cervix and/or uterus, of the donor subject. The vaginal composition of the invention that is administered to the recipient subject in need thereof thus comprises the entirety of the ecological niche present in the genitourinary tract, e.g., the vaginal tract or cervicovaginal tract, of the donor subject.

In some embodiments, the vaginal composition may comprise a pharmaceutically acceptable carrier or diluent, optionally wherein the diluent is saline.

The vaginal composition may be administered in a single dose or in multiple doses to the subject.

In some embodiments, the subject receives a single dose of the vaginal composition of the invention. In some embodiments, the subject receives multiple doses of the vaginal composition of the invention. The multiple doses comprise a first dose and one or more subsequent doses.

In case of multiple doses, the doses can be administered on consecutive days or non-consecutive days. In some embodiments, the subsequent doses are administered if the genitourinary tract microbiota is determined to be dysbiotic. In some embodiments, two to five doses of the vaginal composition are administered to the subject, wherein each dose is administered to the subject on each of two to five consecutive days. One dose of the vaginal composition may be administered to the subject each time the subject reverts to a dysbiotic microbiota of the genitourinary tract. If the subject stably maintains a Lactobacilli-dominant microbiota, multiple doses of the vaginal compositions may not be required.

The vaginal composition for the claimed therapeutic uses comprises at least 1×10⁴ colony forming units (CFUs). Further, a dose of the vaginal composition may comprise 1×10³ to 1×10² colony forming units (CFUs).

The present invention provides the vaginal composition for use of treating RPL In a subject in need thereof by reverting the dysbiotic genitourinary tract, e.g., a dysbiotic vaginal or cervicovaginal microbiota, to a healthy, lactobacillus-dominant microbiota.

The vaginal composition for the treatment of RPL therefore comprises administering the vaginal composition repeatedly until the subject's dysbiotic vaginal microbiota is lactobacillus-dominant, e.g., comprises at least 60%, 70%, 80%, 90% or 95% lactobacillus species. In embodiments, the lactobacillus species are selected from Lactobacillus crispatus, Lactobacillus iners, Lactobacillus jensenii, and or Lactobacillus gasseri.

The Lactobacilli comprised in the vaginal composition of the invention are capable of stably engrafting (e.g., colonizing) the subject's vaginal or uterine cavity for a time period of at least 1 month, 2 months, 3 months, 4 months, 5 months, 6 months, 12 months, 18 months, 24 months or longer.

The Lactobacilli comprised in the vaginal composition of the invention are capable of stably engrafting (e.g., colonizing) the subject's vaginal or uterine cavity for a time period of at least the entire pregnancy. In a preferred embodiment, the “entire pregnancy” comprises conception and/or live birth.

In some embodiments, about 80-99.9% of all detectable bacterial species of the vaginal composition consist of Lactobacillus crispatus.

In some embodiments, about 80-99.9% of all detectable bacterial species of the vaginal composition consist of
(a) Lactobacillus crispatus;
(b) Lactobacillus iners;
(c) Lactobacillus crispatus and Lactobacillus iners;
(d) Lactobacillus crispatus and Lactobacillus jensenii;
(e) Lactobacillus crispatus, Lactobacillus iners and Lactobacillus jensenii;
(f) Lactobacillus crispatus, Lactobacillus gasseri and Lactobacillus jensenii;
(g) Lactobacillus iners and Lactobacillus jensenii;
(h) Lactobacillus iners, Lactobacillus jensenii, and Lactobacillus gasseri;
(i) Lactobacillus iners and Lactobacillus gasseri; or
(j) Lactobacillus crispatus, Lactobacillus iners, Lactobacillus jensenii, and Lactobacillus gasseri.

In some embodiments of (c) to (j), Lactobacillus crispatus, Lactobacillus iners, Lactobacillus jensenii, Lactobacillus gasseri may be present in greater relative quantity than one or more of the other vaginal lactobacilli present in the vaginal composition.

In some embodiments, less than 10% of all detectable bacterial species of the dysbiotic vaginal microbiota in the genitourinary tract of the subject belong to the genus Lactobacillus, and at least 20%, 30%, 40%, 50%, 60%, 70% or more of all detectable bacterial species of the dysbiotic vaginal microbiota are pathogens or pathobionts comprising Gardnerella spp., Atopobium spp., and/or Prevotella spp.

The vaginal composition of the invention can be present in any form, including but not limited to a form of a suspension, spray, gel, cream, ointment, semi-solid foam, film, powder, capsule, solution for lavages or douches, ovules, a vaginal insert, tampon, tablets or a microencapsulated product.

The vaginal composition comprises an effective amount to treat RPL by reverting the dysbiotic vaginal microbiota in the genitourinary tract of the subject. The effective amount may comprise at least about 10⁶ viable bacterial cells, preferably at least about 10⁷ viable bacterial cells.

The vaginal composition may comprise further lactobacilli other than Lactobacillus crispatus, Lactobacillus iners, Lactobacillus jensenii, or Lactobacillus gasseri, wherein the further lactobacilli are present in a concentration of about 0.01-1% of all detectable bacterial species of the vaginal composition, optionally wherein the further lactobacilli are Atopobium spp., Bifidobacterium vaginale, Fannyhessea vaginae, and/or Prevotella spp.

The vaginal composition can be obtained by a culture-independent method.

In a further aspect, the invention provides a method of producing a vaginal composition. The method may comprise
a. Processing a microbiota sample from a donor genitourinary tract, e.g. a vaginal mucosal sample or cervicovaginal secretion, comprising urogenital microbes and vaginal mucosal fluid from the vaginal cavity of a healthy donor subject in a centralized processing facility,
b. Assessing the absence of one or more pathogens,
c. Assessing viability and/or quantity of the urogenital microbes, and
d. Releasing the vaginal composition comprising the processed vaginal mucosal sample from quarantine for use in an assisted reproductive procedure, if a predetermined level is obtained in step (b) and (c).

The method of obtaining the vaginal composition may further comprise one, two, three, four or all of
i. Adding at least one pharmaceutically acceptable diluent, excipient or carrier;
ii. Adjusting the pH, osmolarity and/or viscosity of the vaginal mucosal fluid;
iii. Adding one or more cryoprotectants (e.g., for freezing) and/or one or more lyoprotectants (e.g., for drying);
iv. Formulating the processed mucosal fluid into a dosage form comprising a powder, a solid, a semi-solid, or a liquid:
v. Partitioning the vaginal mucosal fluid into discrete units, each unit comprising an effective dose of urogenital microbes, wherein the effective dose of urogenital microbes comprises at least 10⁵ colony forming units (CFU), preferably at least 10⁶ colony forming units (CFU), more preferably at least 10⁷ colony forming units (CFU);
vi. Storing the refrigerated, frozen or dried vaginal mucosal fluid sample or processed vaginal composition under quarantine;
vii. Holding the refrigerated, frozen or dried vaginal mucosal fluid sample or processed vaginal composition under quarantine until any completion of any combination of (a) testing the donor to exclude the substantial presence of one or more transmissible pathogens, (b) confirming the composition and viability of the urogenital microbes comprised, or (c) further confirming the health of the donor by a plurality of post-screening tests;
viii. Standardizing the cell count and/or the quantity or concentration of the urogenital microbes comprised in the composition, optionally by adding an inert filler; and
ix. Releasing the refrigerated, frozen or dried mucosal fluid sample or processed vaginal composition from quarantine to define the vaginal composition.

The resulting vaginal composition may comprise
(i) One, two, three or four bacterial species from the genus Lactobacillus which comprise >80-99.9% of all detectable bacterial species of the vaginal composition, optionally selected from Lactobacillus crispatus, Lactobacillus iners, Lactobacillus jensenii, Lactobacillus gasseri;
(ii) Less than 5% of Gardnerella spp., Atopobium spp., and Prevotella spp.; and optionally,
(iii) Vaginal transudate and/or mucus, optionally wherein the mucus is cervicovaginal mucus.

In one example, the therapeutic methods using vaginal compositions or pharmaceutical compositions comprising the same, can be used to treat RPL in women who are trying to successfully give birth to a viable, live neonate, and who have previously lost two or more pregnancies (or suffered two or more miscarriages).

In another example, the therapeutic methods using vaginal compositions or pharmaceutical compositions comprising the same can be used to treat RPL in women who have been diagnosed with antiphospholipid syndrome (APS), thrombophilia, coeliac disease, uterine abnormalities, hyperprolactinaemia, increased inflammation, thyroid malfunction, polycystic ovary syndrome (PCOS), or aberrant insulin metabolism. Administration of the vaginal composition of the invention or pharmaceutical composition comprising the same increases pregnancy outcomes, such as live births.

In another example, the therapeutic methods using vaginal compositions or pharmaceutical compositions comprising the same can be used to treat RPL in women exhibiting endometriosis undergoing an IVF procedure.

In another example, the therapeutic methods using vaginal compositions or pharmaceutical compositions comprising the same can be used to treat RPL in women who exhibit one or more of endometriosis or another condition associated with RPL and increased risk of failed pregnancies, such as, e.g., antiphospholipid syndrome (APS), thrombophilia, coeliac disease, uterine abnormalities, hyperprolactinaemia, increased inflammation, thyroid malfunction, polycystic ovary syndrome (PCOS), or aberrant insulin metabolism. In embodiments, RPL is associated with PCOS. In embodiments, RPL is associated with APS. In embodiments, RPL is associated with thrombophilia. In embodiments, RPL is associated with coeliac disease. In embodiments, RPL is associated with uterine abnormalities. In embodiments, RPL is associated with hyperprolactinemia. In embodiments, RPL is associated with increased inflammation. In embodiments, RPL is associated with thyroid malfunction. In embodiments, RPL is associated with PCOS. In embodiments, RPL is idiopathic.

In embodiments, the therapeutic methods using vaginal compositions or pharmaceutical compositions comprising the same can be used to treat microbial dysbiosis in the reproductive (e.g., uterine and vaginal) tract of a woman who has experienced repeated pregnancy loss, such as, two or more failed pregnancies.

It has been suggested that the uterine and/or vaginal microbiome may be associated with poor pregnancy outcomes, e.g., in RPL patients (Reprod Biomed Online. 2022 November; 45(5):1021-1031. doi: 10.1016/j.rbmo.2022.06.008.). In embodiments, dysbiosis is associated with a pro-inflammatory tone of the reproductive (e.g., uterine and vaginal) tract, e.g., with the presence of (local) inflammation in the reproductive tract, e.g., as can be measured by one or more pro-inflammatory biomarkers. The pro-inflammatory (local) environment (e.g., the vaginal and/or endometrial epithelium) may render the endometrial surface less receptive and supportive of promoting embryonal growth and viability during pregnancy. Thus, the vaginal and endometrial microbiome might impact the environment for embryo growth and viability, and may contribute to pregnancy success (e.g., healthy microbiome) or failure, e.g., miscarriage (dysbiotic microbiome, e.g., promoting inflammation).

In embodiments, the therapeutic methods using vaginal compositions or pharmaceutical compositions comprising the same can be used to treat RPL, e.g., by promoting embryo viability in a subject exhibiting a dysbiosis in the reproductive tract, e.g., a subject harboring a dysbiotic microbiome that is, e.g., associated with—or causative of—a pro-inflammatory host response in the reproductive tract. In embodiments, the subject undergoes (or is planning to undergo) in vitro fertilization (IVF). In embodiments, the vaginal compositions are effective in modulating the uterus, thereby increasing pregnancy success. In embodiments, the rate of a successful pregnancy is increased, e.g., an increased rate of live birth and/or reduced rate of miscarriages. This reduces the associated cost of specialized care and the emotional burden that is caused by repeated stillbirth. In embodiments, the number of pregnancies required to achieve live birth of a neonate is reduced when compared to a control group not receiving the vaginal compositions of the invention. This can be assessed over a population of subjects, e.g., a plurality of subjects with RPL trying to give live birth. A reduction in the number of required care associated with stillbirths significantly affects health care costs, but more importantly reduces the emotional strain on the people attempting to build a family. In embodiments, the therapeutic methods using vaginal compositions or pharmaceutical compositions comprising the same can be used to decreases the likelihood of an undesirable reproductive outcome, including miscarriages and stillbirths or neonatal death, which are hallmarks of RPL. It is thought that healthy vaginal microbiota balance pro-inflammatory processes that are triggered by the fetus' implantation, and anti-inflammatory, tolerogenic processes that cause the embryo to implant into the uterine wall and allow pregnancy to proceed and succeed. In embodiments, the therapeutic methods using vaginal compositions or pharmaceutical compositions comprising the same can be used to reverse the dysbiosis and provide a supportive environment for the growth of the embryo.

The present invention discloses a vaginal composition for use in a method of treating RPL in a subject in need thereof, wherein vaginal dysbiosis in the genitourinary tract of the subject is reversed to a lactobacilli-dominant microbiota. In some embodiments, a vaginal composition for use in a method of treating dysbiosis in the genitourinary tract of a subject having RPL and exhibiting a dysbiotic microbiota in the genitourinary tract comprises administering to the subject an effective amount of the vaginal composition to treat the dysbiosis in the genitourinary tract, wherein the vaginal composition comprises: (i) one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) less than 5% of Gardnerella spp., Atopobium spp., and Prevotella spp., and (iii) vaginal transudate and/or mucus, optionally wherein the mucus is cervicovaginal mucus.

Some aspects of the present invention disclose a composition for use in a method of promoting embryo growth, prolonged embryo survival and/or prolonged pregnancy in a population of subjects having RPL, wherein each of the subjects in the population exhibits a dysbiotic microbiota in the genitourinary tract. In some embodiments, a vaginal composition for use in a method of promoting embryo growth and survival in a population of subjects having RPL, wherein each of the subjects in the population exhibits a dysbiotic microbiota in the genitourinary tract comprises administering to each of the subjects an effective amount of the vaginal composition to the subject's genitourinary tract to promote successful pregnancy outcome (e.g., live birth), wherein the vaginal composition comprises: (i) one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; (ii) less than 5% of Gardnerella spp., Atopobium spp., and Prevotella spp.; and (iii) vaginal transudate and/or mucus, optionally wherein the mucus is cervicovaginal mucus; and wherein embryo growth, embryo survival and/or the rate of live birth is increased in the population of subjects being administered the vaginal composition relative to a control population.

In some embodiments, the subject having RPL is exhibiting one or more pathological conditions, or the population of subjects having RPL is a population of subjects exhibiting one or more conditions associated with RPL. The one or more conditions associated with RPL may be selected from the group consisting of: antiphospholipid syndrome (APS), thrombophilia, coeliac disease, uterine abnormalities, hyperprolactinemia, increased inflammation, thyroid malfunction, polycystic ovary syndrome (PCOS), and aberrant insulin metabolism. In some embodiments, the one or more conditions associated with RPL is antiphospholipid syndrome (APS). In some embodiments, the one or more conditions associated with RPL is thrombophilia. In some embodiments, the one or more conditions associated with RPL is coeliac disease. In some embodiments, the one or more conditions associated with RPL is uterine abnormalities. In some embodiments, the one or more conditions associated with RPL is hyperprolactinemia. In some embodiments, the one or more conditions associated with RPL is increased inflammation. In some embodiments, the one or more conditions associated with RPL is aberrant insulin metabolism. In some embodiments, the one or more conditions associated with RPL is polycystic ovary syndrome (PCOS).

In some embodiments, the method increases the rate of successful pregnancies in a population of subjects having RPL, relative to a control population. In some embodiments, the population of subjects comprises at least 10, at least 20 or at least 40 subjects.

In some embodiments, the subject is an asymptomatic dysbiotic subject, or the population of subjects is a population of asymptomatic dysbiotic subjects.

The subject may be 15-34 years of age. In some embodiments, a subject who is 15-34 years of age has been unable to carry a successful pregnancy resulting in live birth. The subject may be 35-50 years of age or 51-75 years of age. In some embodiments, a subject who is 34-75 years of age has been unable to carry a successful pregnancy resulting in live birth.

The subject may be 15-44 years of age, 35-44 years of age, or 45 or more years of age. The subject may be 25-37 years of age or 38-45 years of age.

In some embodiments, the subject is a pre-menopausal subject.

In some embodiments, the subject (e.g., a subject having RPL) is undergoing an assisted reproductive procedure, or the population of subjects is a population of subjects undergoing an assisted reproductive procedure. In some embodiments, the assisted reproductive procedure is in vitro fertilization. In some embodiments, the subject is undergoing intrauterine insemination or treatments to stimulate egg production, or the population of subjects is a population of subjects undergoing intrauterine insemination or treatments to stimulate egg production.

In some embodiments, the vaginal composition is formulated into a dosage form; wherein the dosage form comprises an effective amount of the composition in one or more discrete units, wherein the effective amount is predetermined.

In some embodiments, the subject is not treated with an antibiotic prior to administration of the composition. In some embodiments, the composition is administered to the vaginal cavity of the subject. In some embodiments, prior to administering the composition, a vaginal wash is performed and/or wherein the vaginal wash is subsequently rinsed with saline and/or lactic acid.

The vaginal wash may be performed with saline, lactic acid or an antiseptic agent, optionally wherein the antiseptic agent is povidone-iodine or chlorohexidine.

In some embodiments, the subject has been diagnosed with bacterial vaginosis or a vaginal infection, and the subject is pre-treated with an antibiotic prior to administration of the composition. In some embodiments, the subject has not been diagnosed with bacterial vaginosis or a vaginal infection, wherein the subject is not pre-treated with an antibiotic prior to administration of the composition.

In some embodiments, the subject exhibits endometrial and/or vaginal inflammation. In some embodiments, the subject has elevated vaginal levels of pro-inflammatory or inflammasome-associated cytokines. The pro-inflammatory or inflammasome-associated cytokines may comprise at least one of IL-1α, IL-1β, IL-2, IL-5, IL-6, IL-8, IL-12, IL-15, IL-17, IL-18, IP-10, MCP-1, TNFα, HIF-1α, COX-2 and VEGF, optionally wherein the pro-inflammatory or inflammasome-associated cytokines comprise at least one of IL-1, IL-1α, IL-1β, IL-6, or IL-18, or any combination thereof. In some embodiments, the subject has elevated vaginal levels of Th1 cytokines. The Th1 cytokines may comprise at least one of IFNγ, IL-12, STAMBP or TNF. In some embodiments, the subject is further characterized by reduced vaginal levels of IGF-1, IL-4, IL-10, TGFβ and/or CCL5.

In some embodiments, after administration of the composition to the subject one or more of: (i) the elevated vaginal levels of pro-inflammatory or inflammasome-associated cytokines in the subject are reduced; (ii) the elevated vaginal levels of Th1 cytokines in the subject are reduced; and/or (iii) the reduced vaginal levels of IGF-1, IL-4, IL-10, TGFβ and/or CCL5 in the subject are increased.

In some embodiments, the vaginal composition comprises vaginal transudate and/or mucus, optionally wherein the mucus is cervicovaginal mucus. In some embodiments, the vaginal composition was obtained from a donor subject and further wherein the vaginal composition comprises substantially all detectable bacterial, viral, fungal species, and/or metabolites, that are present in the genitourinary tract, e.g. the vagina, cervix and/or uterus, of the donor subject.

In some embodiments, the composition further comprises a pharmaceutically acceptable carrier or diluent, optionally wherein the diluent is saline.

In some embodiments, the method further comprises transferring of a human embryo to the subject. In some embodiments, the human embryo has not previously been frozen. In some embodiments, the human embryo has previously been frozen. In some embodiments, the human embryo is transferred after 1 week, 2 weeks, 3 weeks, or up to 12 weeks after administering the composition.

In some embodiments, the method further comprises one or more of (a) determining a partial or complete reversal of the subject's dysbiotic vaginal microbiota after administration of the vaginal composition such that the subject's vaginal microbiota comprises (i) one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the vaginal composition; and (ii) less than 5% of Gardnerella spp., Atopobium spp., and Prevotella spp.; (b) determining a reduction of pro-inflammatory or inflammasome-associated cytokines (e.g., IL-1, IL-1α, IL-1β, IL-6, or IL-18); and/or (c) determining a reduction of Th1 cytokines (e.g., IFNγ, IL-12, STAMBP or TNF).

In some embodiments, a single dose is administered to the subject or multiple doses of the vaginal composition are administered to the subject having RPL. In some embodiments, multiple doses of the vaginal composition are administered to the subject over consecutive days. In some embodiments, three doses of the vaginal composition are administered to the subject, wherein one dose is administered to the subject on each of three consecutive days. In some embodiments, 1-3 doses of the vaginal composition are administered to the subject on each of three consecutive days.

In some embodiments, a dose of the vaginal composition is at least 1×10⁴ colony forming units (CFUs). In some embodiments, a dose of the vaginal composition is 1×10³ to 1×10¹² colony forming units (CFUs).

In some embodiments, the vaginal composition is administered repeatedly until the subject's dysbiotic vaginal microbiota is lactobacillus-dominant, e.g., comprises at least 60%, 70%, 80%, 90% or 95% lactobacillus species, wherein the species are selected from Lactobacillus crispatus, Lactobacillus iners, Lactobacillus jensenii, and or Lactobacillus gasseri.

In some embodiments, the Lactobacilli comprised in the vaginal composition are capable of stably engrafting (e.g., colonizing) the subject's vaginal or uterine cavity for a time period of at least 1 month, 2 months, 3 months, 4 months, 5 months, 6 months, 12 months, 18 months, 24 months or longer.

In some embodiments, about 80-99.9% of all detectable bacterial species of the vaginal composition of the invention consist of Lactobacillus crispatus. In some embodiments, about 80-99.9% of all detectable bacterial species of the vaginal composition consist of
(a) Lactobacillus crispatus;
(b) Lactobacillus iners;
(c) Lactobacillus crispatus and Lactobacillus iners;
(d) Lactobacillus crispatus and Lactobacillus jensenii;
(e) Lactobacillus crispatus, Lactobacillus iners and Lactobacillus jensenii;
(f) Lactobacillus crispatus, Lactobacillus gasseri and Lactobacillus jensenii;
(g) Lactobacillus iners and Lactobacillus jensenii;
(h) Lactobacillus iners, Lactobacillus jensenii, and Lactobacillus gasseri;
(i) Lactobacillus iners and Lactobacillus gasseri; or
(j) Lactobacillus crispatus, Lactobacillus iners, Lactobacillus jensenii, and Lactobacillus gasseri.

In some embodiments, Lactobacillus crispatus, Lactobacillus iners, Lactobacillus jensenii, Lactobacillus gasseri may be present in greater relative quantity than one or more of the other vaginal lactobacilli present in the preparation.

In some embodiments, less than 10% of all detectable bacterial species of the dysbiotic vaginal microbiota in the genitourinary tract of the subject belong to the genus Lactobacillus, and at least 20%, 30%, 40%, 50%, 60%, 70% or more of all detectable bacterial species of the dysbiotic vaginal microbiota are pathogens or pathobionts comprising Gardnerella spp., Atopobium spp., and/or Prevotella spp.

In some embodiments, the vaginal composition is in the form of a suspension, spray, gel, cream, ointment, semi-solid foam, film, powder, capsule, solution for lavages or douches, ovules, a vaginal insert, tampon, tablets or a microencapsulated product.

In some embodiments, the vaginal composition comprises at least about 10⁶ viable bacterial cells, preferably at least about 10⁷ viable bacterial cells.

In some embodiments, the vaginal composition further comprises further lactobacilli other than Lactobacillus crispatus, Lactobacillus iners, Lactobacillus jensenii, or Lactobacillus gasseri, wherein the further lactobacilli are present in a concentration of about 0.01-1% of all detectable bacterial species of the preparation, optionally wherein the further lactobacilli are Lactobacillus helveticus, Lactobacillus amylovorus, Lactobacillus johnsonii, Atopobium spp., Bifidobacterium vaginale, Fannyhessea vaginae, and/or Prevotella spp.

In some embodiments, the vaginal composition is obtained by a culture-independent method.

Some aspects of the present invention disclose a method of producing the vaginal composition of the invention, said method comprising processing a microbiota sample from a donor genitourinary tract, e.g. a vaginal mucosal sample or cervicovaginal secretion, comprising urogenital microbes and vaginal mucosal fluid from the vaginal cavity of a healthy donor subject in a centralized processing facility,
a. Assessing the absence of one or more pathogens,
b. Assessing viability and/or quantity of the urogenital microbes, and
c. Releasing the vaginal composition comprising the processed vaginal mucosal sample from quarantine for use in an assisted reproductive procedure, if a predetermined level is obtained in step (b) and (c).

In some embodiments, the method of obtaining the vaginal composition further comprises one, two, three, four or all of
e. Adding at least one pharmaceutically acceptable diluent, excipient or carrier;
f. Adjusting the pH, osmolarity and/or viscosity of the vaginal mucosal fluid;
g. Adding one or more cryoprotectants (e.g., for freezing) and/or one or more lyoprotectants (e.g., for drying);
h. Formulating the processed mucosal fluid into a dosage form comprising a powder, a solid, a semi-solid, or a liquid:
i. Partitioning the vaginal mucosal fluid into discrete units, each unit comprising an effective dose of urogenital microbes, wherein the effective dose of urogenital microbes comprises at least 10⁵ colony forming units (CFU), preferably at least 10⁶ colony forming units (CFU), more preferably at least 10⁷ colony forming units (CFU);
j. Storing the refrigerated, frozen or dried vaginal mucosal fluid sample or processed vaginal composition under quarantine;
k. Holding the refrigerated, frozen or dried vaginal mucosal fluid sample or processed vaginal composition under quarantine until any completion of any combination of (a) testing the donor to exclude the substantial presence of one or more transmissible pathogens, (b) confirming the composition and viability of the urogenital microbes comprised, or (c) further confirming the health of the donor by a plurality of post-screening tests;
l. Standardizing the cell count and/or the quantity or concentration of the urogenital microbes comprised in the preparation, optionally by adding an inert filler; and
m. Releasing the refrigerated, frozen or dried mucosal fluid sample or processed vaginal composition from quarantine to define the vaginal composition.

In some embodiments, wherein the vaginal composition comprises (i) one, two, three or four bacterial species from the genus Lactobacillus, selected from Lactobacillus crispatus, Lactobacillus iners, Lactobacillus jensenii, Lactobacillus gasseri, which comprise >80-99.9% of all detectable bacterial species of the preparation; and (ii) less than 5% of Gardnerella spp., Atopobium spp., and Prevotella spp.; and (iii) vaginal transudate and/or mucus, optionally wherein the mucus is cervicovaginal mucus.

### BRIEF DESCRIPTION OF THE FIGURES

FIG. 1 is a schematic representation of the donor program to produce vaginal compositions that are used in the therapeutic methods of the invention. Donors were selected based on microbiome sequencing, a medical examination, and the absence of certain diseases. Testing was performed both before and after the donation visits. All samples provided by the donor were subjected to quality control (see, e.g., FIG. 5). Samples were released only when donor and samples passed all requirements.

FIG. 2 shows the distribution of vaginal microbiomes from a cohort of 96 subjects that were assessed by shotgun DNA sequencing to identify suitable donors from which to obtain cervicovaginal secretions. Relative abundance of bacteria was measured, and subjects were classified as “healthy”, “dysbiotic” and “undefined” using the classification parameters indicated.

FIG. 3 shows stacked bar graphs of relative bacterial abundance in the donors from the cohort of 96 subjects (see, FIG. 2) with a healthy vaginal microbiome (n=61), as assessed by shotgun sequencing. All donor microbiomes in this graph contain at least 80% of vaginal Lactobacillus species (Lactobacillus crispatus, Lactobacillus gasseri, Lactobacillus jensenii, and Lactobacillus iners) or mixtures thereof, and less than 5% selected pathogens (Atopobium spp., Prevotella spp., B. vaginale, and F. vaginae) or mixtures thereof.

FIG. 4 shows stacked bar graphs of relative bacterial abundance in the donors from the cohort of 96 subjects (see, FIG. 2) with a dysbiotic vaginal microbiome (left, n=27) or undefined microbiome (right, n=8) as assessed by shotgun sequencing. Dysbiotic microbiomes contain at least 20% species from selected vaginal pathogens (Atopobium spp., Prevotella spp., B. vaginale, and F. vaginae) or mixtures thereof, and less than 10% of vaginal Lactobacillus species (Lactobacillus crispatus, Lactobacillus gasseri, Lactobacillus jensenii, and Lactobacillus iners) or mixtures thereof.

FIG. 5 shows a flowchart depicting an exemplary procedure to obtain cervicovaginal secretions (CVS) and processing thereof for preparing the vaginal composition of the invention. The secretions were split into samples for characterization and quality control (including, e.g., viability count, DNA sequencing and pathogen screening) and processing into a vaginal composition for administration.

FIGS. 6A-6I shows stacked bar graphs of four main Lactobacillus species present in the vaginal compositions from 9 donors (6A-6I) as assessed by shotgun sequencing. The results from eight representative donation visits are shown for each donor. Where present, the vertical line indicates a new round of visits as recurring donor.

FIGS. 7A and 7B show stacked bar graphs of relative bacterial abundance in two exemplary recipients (7A-7B) of a vaginal composition and their respective donors, as measured by shotgun sequencing. All recipient subjects were enrolled based on their microbiome at screening; a baseline microbiome sample was taken just prior to administration of the vaginal compositions. The reversal or dissolution of the dysbiotic vaginal microbiome was stable for at least 6 months (final data for recipient 1, FIG. 7A) and at least 3 months (interim data for recipient 2, FIG. 7B).

FIGS. 8A-8D show three different PCA plots for inflammatory markers comparing women with a dysbiotic and healthy microbiome by proteomics (FIG. 8A), transcriptomics (FIG. 8B) and metabolomics (FIG. 8C). A. PCA plot for inflammatory proteins (healthy: n=41, dysbiotic: n=19), where each dot corresponds to one woman; 92 inflammation panel markers were used as variables. The asterisk (*) indicates a woman with a healthy microbiome who might have an unrecognized infection such as, e.g., Candida. B. PCA plot for inflammatory mRNA molecules in dysbiotic and healthy subjects (healthy: n=4, dysbiotic: n=6) using Nanostring analysis. 579 inflammatory markers were used as variables. C. PCA plot for small molecules associated with inflammation in dysbiotic and healthy subjects (healthy: n=5, dysbiotic: n=7) using MS-omics. 879 inflammatory markers were used as variables. D. Box and whisker plot of selected inflammatory markers, which are associated with bacterial vaginosis.

FIG. 9 shows plots of inflammatory markers from the inflammation panel described in FIG. 8A in dysbiotic recipient pre- and post-administration with a vaginal composition. PCA plot shows the shift in inflammation state in the recipient pre- and post-treatment with the vaginal composition, relative to the healthy donor samples and with a subset of the screening cohort for comparison. The 92 inflammation panel markers were used as variables.

FIG. 10 shows stacked bar graphs of relative bacterial abundance. The two donor samples (both obtained from same donor subject) used for administration to the recipient subject (indicated by black triangle) are shown on the left. All remaining bar graphs depict the relative bacterial abundance of the recipient subject prior to and after administration of the donor samples. ‘Baseline’ indicates the recipient's relative bacterial abundance prior to receiving the first donor sample.

FIG. 11 is a schematic representation of the recipient's data, showing the relative abundance of L. crispatus in [%](top graph), vaginal pH (middle graph), and cervical length (bottom graph).

FIG. 12 shows a heatmap illustrating pairwise Manhattan distances between SNV (single nucleotide variant) profiles generated from metagenomic sequencing of CVS samples. The colorimetric scale indicates close to distant profiles from black to white respectively. The recipient material labels refer to the time since the first administration, at which samples were collected for metagenomic sequencing. The donor materials include pairwise distances with the two samples obtained from the same donor used for samples 1 and 2, as well as three other samples from three separate donors that were not used for administration but served as controls.

FIG. 13 shows a line graph illustrating specific longitudinal trends of the same pairwise Manhattan distances between SNV profiles used to generate the heatmap (FIG. 3). The horizontal axis labels refer to the time since the first administration. The vertical axis indicates close to distant profiles from low to high respectively. Each line illustrates the pairwise distance between the recipient samples and different individual donor samples over time. Samples obtained from the same donor that were used for administration are labeled Administration 1 and 2, and three other samples from three separate donors that were not used for administration are labeled as Control 1, 2 and 3.

### DETAILED DESCRIPTION

The present invention relates to compositions, devices, and kits comprising vaginal compositions and methods of making and using the same. Provided herein are vaginal compositions and compositions for modulation of the vaginal microbiota for treatment of RPL The applicant has developed a safe and efficacious process to produce vaginal compositions and provides devices and methods for administering and using same, e.g., to restore or maintain a healthy human vaginal microbiota balance in the genitourinary tract, to treat dysbiosis and/or inflammatory conditions, e.g., in women exhibiting RPL, thereby treating the RPL and leading to increased live birth pregnancy outcomes.

### Microbiome in the Reproductive Tract

The reproductive tract of placental mammals, including humans, comprises unique structures such as the vagina and the uterus. Distinct bacterial communities exist throughout the reproductive tract. The vaginal bacterial community is the most well described, however, bacteria have been detected in the upper reproductive tract as well, forming a continuum of microbiotas changing from the vagina to the ovaries. This continuum may exist despite the barrier properties of cervicovaginal mucus that is capable of “trapping” or slowing the diffusion of microbes or microbe-sized particles through steric and adhesive interactions. This microbial continuity along the reproductive tract (vagina, cervix, uterus, and fallopian tubes) is present in women of reproductive age.

A healthy vaginal flora is characterized by an acidic environment inhabited predominantly by lactic acid bacteria, primarily species of Lactobacillus (residing in the vaginal microbial niche).

The microbial composition in healthy women can differ, though it is typically dominated by one of four Lactobacillus species: L. crispatus, L. iners, L. gasseri, L. jensenii, and mixtures thereof.

A healthy vagina of a women of child-bearing age is estimated to be dominated by 10⁷-10¹ colony forming units of lactic acid producing bacteria (e.g., Lactobacillus) per gram of fluid. The species distribution differs between women of different geographical background, race (e.g., Asian, white women, black, Hispanic), age, lifestyle and the like. The composition of the vaginal flora is also influenced by which specific strains and/or species the woman has inherited from her mother and/or which strains and/or species have migrated from her digestive tract to the urogenital tract. Healthy, fertile women present with a pH of about 3 to 5.5 (more specifically between pH 3.5 and 4.5) in the vagina, primarily as a result of lactic acid production. Vaginal pH undergoes physiological changes from birth to menopause. The increase of vaginal pH above 4.0-4.5 is detrimental for the survival of Lactobacillus bacteria, but not for other microorganisms. The vaginal lactobacilli are believed to have a protective effect against vaginal colonization by pathogenic microorganisms (e.g., yeast (Candida albicans), Trichomonas vaginalis, Neisseria gonorrhoeae, and Chlamydia trachomatis, and viruses, e.g., HV, HSV-2, and various anaerobes) and prevent the vaginal establishment of, for instance, bacteria that are present in the colon, such as Gardnerella vaginalis, Mobiluncus, Bacteroides, Prevotella and Escherichia coli.

Several factors may contribute to the disturbance of the vaginal flora. Factors may include, a) use of antibiotics to kill pathogenic bacteria which can lead to significantly reduced levels of lactobacilli in the vagina; b) hormonal changes, in particular changes in estrogen levels, which are observed in several phases of a woman's life (e.g., puberty, pregnancy, childbearing age, pre- and post-menopause); estrogen levels are thought to be associated with Lactobacillus levels (dominance) in the vagina; c) sexual intercourse, which can be associated with pH increases (semen generally is alkaline) that may disturb the vaginal flora, because bacteria other than lactobacilli may start to flourish once the vaginal pH increases; d) use of medications, e.g., chemotherapeutics or antimycotics; e) use of birth control products; f) during menstruation; g) insufficient hygiene (e.g., promoting undesirable spread of the microorganisms from rectum to the urogenital area); h) general health status, such as, e.g., being diabetic.

Disturbance of the vaginal flora may lead to vaginal dysbiosis and vaginal disorders, e.g., candidiasis and bacterial vaginosis, which are two common vaginal disorders that affect women worldwide. Bacterial vaginosis is believed to be the result of displaced vaginal lactic acid producing bacteria which are replaced by a range of unwanted species such as Gardnerella vaginalis, Bacteroides, Mobiluncus, Prevotella, and Mycoplasma hominis. Vaginal infections are most often associated with one or more of: Escherichia, Enterococcus, Pseudomonas, Proteus, Klebsiella, Streptococcus, Staphylococcus, Gardnerella, Ureaplasma, Bacteroides, Peptococcus, Neisseria, Serratia, Corynebacterium, Clostridium, and Candida.

Vaginal lactobacilli predominance is thought to play an important role in resistance to infection via production of lactic acid and acidification of the vagina and by production of other antimicrobial products, such as, e.g., hydrogen peroxide. The presence of lactobacilli in the vagina has been linked to decreased frequencies of bacterial vaginosis, yeast vaginitis and sexually transmitted pathogens, including Neisseria gonorrhea, Chlamydia trachomatis, and Trichomonas vaginalis. Lactobacillus dominance varies among ethnic groups (they are thought to be very predominant in Asian and white women but less so in black and Hispanic women, though they still represent the majority).

Studies have shown that Lactobacillus-depleted communities can be transient, lasting just a few days, while in other instances the depleted communities persist for many weeks. Some women with Lactobacillus-depleted communities remain asymptomatic and healthy. However, such women may be at higher risk for infections and sexually transmitted diseases (STDs), and face challenges getting pregnant, e.g., naturally and/or assisted, e.g., with IVF procedure. In case of IVF, these women often undergo multiple IVF cycles with elevated rates of embryo implantation failures.

### Immune State and Implantation Success

Successful pregnancy is thought to require tightly orchestrated immune interactions between maternal and fetal cells to prevent fetus rejection, while at the same time maintaining effective immune responses in order to protect the mother from harmful pathogens. Several regulatory mechanisms are activated to create a favorable immunological environment for the developing fetus, which can be triggered or suppressed by the microbiota in the vaginal niche of the reproductive tract.

It is thought that a successful pregnancy is dependent on the maternal immune system to establish an implantation window by shifting an initial pro-inflammatory response to a tolerogenic state. Initially, pro-inflammatory cytokines and recruited immune cells regulate the adhesive and non-adhesive molecules involved in trophoblast adhesion to the uterine wall, which is the requisite first step of implantation and, subsequently placentation (Marwood et al., Interleukin-11 and leukemia inhibitory factor regulate the adhesion of endometrial epithelial cells: implications in fertility regulation. Endocrinology. 150, 2915-2923 (2009); McEwan et al., Cytokine regulation during the formation of the fetal-maternal interface: focus on cell-cell adhesion and remodeling of the extra-cellular matrix. Cytokine Growth Factor Rev. 20, 241-249 (2009)). This early inflammation is characterized by an increase in pro-inflammatory cytokines such as, e.g., TNF-α, IL-6, MIP-1β, CX3CL-1, osteopontin, IL-15 and IL-10, secreted by immune and endometrial cells (Boomsma et al., Ovarian stimulation for in vitro fertilization alters the intrauterine cytokine, chemokine, and growth factor milieu encountered by the embryo. Fertil. Steril. 94, 1764-1768 (2010); Gnainsky et al., Local injury of the endometrium induces an inflammatory response that promotes successful implantation. Fertil. Steril. 94, 2030-2036 (2010); Granot et al., Endometrial inflammation and effect on implantation improvement and pregnancy outcome. Reproduction 144, 661-668 (2012); Van Sinderen et al., Preimplantation human blastocyst-endometrial interactions: the role of inflammatory mediators. Am. J. Reprod. Immunol. 69, 427-440 (2013)). The subsequent transition into an anti-inflammatory and pro-tolerogenic immune environment is characterized by an expansion and recruitment of regulatory T cells (Robertson et al., Immunological determinants of implantation success. Int J Dev Biol (2014) 58(2-4):205-17; Robertson et al., Regulatory T cells in embryo implantation and the immune response to pregnancy. J Clin Invest (2018) 128(10):4224-35; Erlebacher et al., Mechanisms of T cell tolerance towards the allogeneic fetus. Nat Rev Immunol (2013) 13(1):23-33)). T-regulatory cells interact with dendritic cells and macrophages to promote decidualization of uterine stromal cells, suppress inflammation, and inhibit effector immunity towards fetal antigens through secretion of IL-10 and TGF-β and a Th-2 anti-inflammatory profile in the endometrium. Uterine natural killer (uNK) cells mediate structural changes in the decidual vasculature that support placental invasion and development. Furthermore, it is thought that uNKs stimulate protolerogenic M2 macrophages and dendritic cells through secretion of IFN-γ and IL-10 (Gonzales et al., Immunogenic Functions Compatible with Pregnancy Progression. PLoS ONE 7(10): e46755 (2012)). It is thought that a Lactobacillus-dominated (healthy) vaginal microbiome may help to modulate the inflammatory response needed for implantation. A dysbiotic microbiome creates a strong pro-inflammatory response which has been shown to be associated with implantation failure. This includes altered antigen presentation and T cell differentiation, proliferation and activation of regulatory cells, as well as the action of hormones, cytokines and other soluble factors. In embodiments, elevated cervico-vaginal cytokines in dysbiotic vaginal niches comprise one or more of IL-1α, IL-1β, IL-2, IL-5, IL-6, IL-8, IL-12, IL-15, IL-17, IL-18, IP-10, MCP-1, TNFα, HIF-1α, COX-2 and VEGF. Dysbiosis is also associated with elevated levels of one or more of, e.g., Eotaxin, IL-10, IL-12p40, IL-17, IL-1RA, sIL-2rα, IL-1a, IL-1β, IL-2, IL-6, IP-10, MCP-1, MIP-1α, MIP-1β, TNFα, HIF-1α, COX-2 and VEGF.

These biomarkers may be produced in response to inflammation in the vagina which can in turn result from the dysbiosis. For example, the cytokine IL-10, which is generally considered to be an anti-inflammatory, regulatory cytokine, can be produced in response to inflammation, e.g., induced by dysbiosis, as a compensatory mechanism. In embodiments, a healthy vaginal microbiome may be associated with one or more of the cervico-vaginal cytokines IGF-1, IL-4, IL-10, TGFβ and CCL5.

The vaginal compositions described herein can be used to treat the (local) inflammation (e.g., of the reproductive tract) associated with the dysbiosis (dysbiotic microbiota). In embodiments, no pre-treatment with antibiotics is necessary to substantially revert the dysbiosis (e.g., to healthy) and/or reduce the inflammatory tone in the reproductive tract, which was not previously possible.

The preparations provided herein comprise not only a substantially complete (e.g., entire) ecosystem of the vaginal microbiota but also sufficient concentrations/doses and/or amount of the vaginal microbiota in the preparation, and may also, in embodiments, include additional substances, such as, e.g., one or more (microbial) metabolites of the healthy vaginal niche, and mucus.

The liberal use or overuse of antibiotics is directly associated with the problem of increasing antibiotic resistance. The present preparations, in embodiments, are not reliant on using antibiotics prior to or during treatment, which is thus a further advancement and advantageous effect. In subjects seeking to undergo IVF procedure that have bacterial vaginosis (BV), a subject may be pretreated with antibiotics in accordance with standard care, which would be prior to administration of the pharmaceutical composition comprising the vaginal composition provided herein.

Successful colonization and engraftment of the lactobacilli comprised in the vaginal compositions may be indicated by one or more of: decreased pH, increased lactic acid content, lower abundance of antibiotic resistance genes, decreased amount of fungal DNA, decreased toxin content, decreased pathogenicity factors, decreased inflammatory cytokines and chemokines, decreased immune cell infiltrates, decreased total bacterial DNA load, decreased total pathogenic DNA load, increased viscoelasticity, increased sialoglycan content, decreased relative or absolute abundance of pathobionts or pathogens, or any combination thereof in the vaginal cavity and the vaginal microbial niche (e.g., when compared to baseline of the same subject (e.g., prior to administration and engraftment) or a non-treated control subject).

In embodiments, the vaginal composition promotes a receptive endometrial lining by affecting a change of the microbiota in the vagina and uterus (e.g., from dysbiotic to healthy), and modulating the inflammation status therein, which in turn increases the chances of successful embryo implantation. Thus, in some embodiments, the vaginal composition promotes embryo implantation in a single subject (e.g., a subject experiencing RPL and/or undergoing an assisted reproductive procedure (e.g., IVF), intrauterine insemination or treatments to stimulate egg production) or in a population of subjects.

Embryo implantation may be determined using any method that is known to skilled persons in the field of fertility and reproductive health. The measure of embryo implantation is typically a binary measurement—the embryo either successfully implants into the uterine tract or the embryo does not implant (e.g., no ‘partial’ implantation). In some embodiments, embryo implantation may be measured using a Beta-Human Chorionic Gonadotropin (beta-CG) test (e.g., at two weeks after embryo transfer or conception). In some embodiments, embryo implantation is determined by identifying whether there is at least one gestational sac (e.g., intrauterine gestational sac) at a defined time point (e.g., 5-6 weeks after embryo transfer or conception). In some embodiments, embryo implantation is determined by identifying whether there is a viable pregnancy (e.g., fetal heartbeat present at a defined time point, e.g., 5-6 weeks after embryo transfer or conception).

### Vaginal Microbiota and Fertility Treatments

In healthy human subjects, the vaginal, cervical and endometrial microbiota harbor microbiota that is dominated by lactobacilli. Women with a healthy, lactobacilli-dominated vaginal microbiota are thought to have a higher success rate for fertility treatments (e.g., in vitro fertilization (IVF), intrauterine insemination or treatments to stimulate egg production) than those with dysbiotic vaginal microbiota (See, e.g., Haahr et al., Abnormal vaginal microbiota may be associated with poor reproductive outcomes: a prospective study in IVF patients. Hum Reprod. 2016 April; 31(4):795-803; Koedooder et al., The vaginal microbiome as a predictor for outcome of in vitro fertilization with or without intracytoplasmic sperm injection: a prospective study. Hum Reprod. 2019 Jun. 4; 34(6):1042-1054; Moreno et al., Endometrial microbiota composition is associated with reproductive outcome in infertile patients. Microbiome. 2022 Jan. 4; 10(1):1). Further, women suffering from bacterial vaginosis (BV) and vaginal dysbiosis have an increased prevalence of diseases or disorders that are associated with inflammation and reduced RPL, such as pelvic inflammatory disease (PID)/endometritis (Wiesenfeld, et al, Lower genital tract infection and endometritis: insight into subclinical pelvic inflammatory disease. Obstet Gynecol. 2002 September; 100(3):456-63; Haggerty, et al, Evaluation and Clinical Health study investigators. Bacterial vaginosis and anaerobic bacteria are associated with endometritis. Clin Infect Dis. 2004 Oct. 1; 39(7):990-5), idiopathic RPL (Campisciano, et al Subclinical alteration of the cervical-vaginal microbiome in women with idiopathic RPL. J Cell Physiol. 2017 July; 232(7):1681-1688; Spandorfer et al., Relationship of abnormal vaginal flora, proinflammatory cytokines and idiopathic RPL in women undergoing IVF. J Reprod Med. 2001 September; 46(9):806-10), secondary RPL (Characterization of the Vaginal Microbiome in Women with RPL and Its Potential Correlation with Hormone Stimulation during In Vitro Fertilization Surgery. mSystems. 2020 Jul. 14; 5(4):e00450-20), tubal factor RPL (Wilson, et al, Rates of bacterial vaginosis in women undergoing in vitro fertilisation for different types of RPL. BJOG. 2002 June; 109(6):714-7), endometriosis (Jiang, et al, Intricate Connections between the Microbiota and Endometriosis. Int J Mol Sci. 2021 May 26; 22(11):5644; Wei, et al, Microbiota composition and distribution along the reproductive tract of women with endometriosis. Ann Clin Microbiol Antimicrob. 2020 Apr. 16; 19(1):15; Perrotta, et al, 2020, Reprod Sci),), and polycystic ovary syndrome (PCOS) (Salah, et al, 2012, Eur J Obst & Gyn and Rep Bio).

Previous attempts to address dysbiosis and BV in the reproductive tract have largely been unsuccessful. Use of antibiotics does not have lasting effects and treatment shows high rates of dysbiosis (and pathogen) recurrence. Various probiotic products have been suggested and tried.

These approaches have had limited success. One major limitation of past approaches has been the use of species typically found in the gut (rhamnosus, acidophilus, reuteri), whereas it was recently demonstrated that in healthy women, typically only 4 species of Lactobacillus (crispatus, iners, gasseri, jensenii) dominate in the human vagina (Gajer et al., 2012; Ravel et al., 2011). One product in development that contains a vaginal-specific Lactobacillus crispatus strain showed only limited therapeutic potential (Hemmerling et al., 2010; Ngugi et al., 2011).

The compositions (e.g., the vaginal composition) and uses described herein overcome these deficiencies by providing and using pharmaceutical compositions comprising a vaginal composition to treat RPL. In embodiments, the compositions and uses described herein improve the rate of successful pregnancies. In embodiments, the rate of embryonic implantation is increased. In embodiments, women undergoing IVF procedure (e.g., women with asymptomatic dysbiosis in the reproductive tract) thus require fewer IVF cycles to become pregnant, e.g., compared to women (e.g., women with asymptomatic dysbiosis in the reproductive tract) not receiving the treatments described herein. In embodiments, the compositions and uses described herein substantially revert a dysbiotic microbiome to a healthy microbiome. In embodiments, the compositions and uses described herein substantially decrease a local (e.g., in the reproductive tract) inflammatory tone (e.g., as measured by pro-inflammatory biomarkers). The vaginal composition comprises substantially all microbe species that are present in a healthy vaginal or urogenital tract (e.g., the reproductive tract of a healthy donor). In embodiments, the pharmaceutical compositions provided herein improve the rate of successful pregnancies, e.g., by reverting vaginal dysbiosis and the associated pro-inflammatory environment.

In some embodiments, the dysbiotic vaginal, cervical or uterine flora is restored or reverted by replenishing or supplementing the disturbed vaginal, cervical or uterine flora with the vaginal composition or a pharmaceutical composition comprising the same described herein.

A subject undergoing fertility treatment (e.g., IVF) may suffer from RPL or sub-fertility. Thus, in some embodiments, the subject undergoing fertility treatments (e.g., IVF) is an infertile subject. In some embodiments, the subject undergoing fertility treatments (e.g., IVF) may have an inflammation-associated RPL condition. An inflammation-associated RPL condition may comprise bacterial vaginosis (BV), pelvic inflammatory disease (PID)/endometritis, tubal factor RPL, endometriosis, or polycystic ovary syndrome (PCOS). In some embodiments, the subject has one or more of the inflammation-associated RPL condition. In some embodiments, the subject has bacterial vaginosis (BV). In some embodiments, the subject has pelvic inflammatory disease (PID). In some embodiments, the subject has tubal factor RPL. In some embodiments, the subject has endometriosis. In some embodiments, the subject has polycystic ovary syndrome (PCOS). In some embodiments, the subject has idiopathic (unexplained) RPL. In some embodiments, the subject has endometrial RPL, which results from endometriosis.

Endometriosis is a chronic inflammatory disease that can cause tissue similar to the lining of the uterus to grow outside of the uterus.

In some embodiments, the subject undergoing fertility treatments (e.g., IVF) is a fertile subject.

A subject who is “undergoing fertility treatments” or “undergoing IVF” may be a subject who is preparing to begin fertility treatments or IVF. For example, a subject exhibiting a dysbiotic microbiota in the genitourinary tract is said to be undergoing IVF if they are administered a vaginal composition prior to beginning IVF treatments.

A subject in need of a vaginal composition as described herein may be an asymptomatic dysbiotic subject. An asymptomatic dysbiotic subject is generally described as a subject that has a dysbiotic vaginal microbiome but is not exhibiting symptoms (e.g., clinical symptoms) of bacterial vaginosis or a vaginal infection (e.g., a vaginal yeast infection). Thus, in some embodiments, the dysbiotic vaginal microbiome of an asymptomatic dysbiotic subject does not comprise detectable or substantial amounts of pathogenic microbes. An asymptomatic dysbiotic subject may have endometriosis, tubal factor RPL or PCOS.

In some embodiments, the subject is an asymptomatic dysbiotic subject, or the population of subjects is a population of asymptomatic dysbiotic subjects.

The subject may be 15-34 years of age. In some embodiments, a subject who is 15-34 years of age has been unable to conceive after 12 months of unprotected sexual intercourse with one or more men. The subject may be 35-50 years of age or 51-75 years of age. In some embodiments, a subject who is 34-75 years of age has been unable to conceive after 6 months of unprotected sexual intercourse with one or more men.

The subject may be 15-44 years of age, 35-44 years of age, or 45 or more years of age. The subject may be 25-37 years of age or 38-45 years of age.

In some embodiments, the subject is a pre-menopausal subject.

In some embodiments, the subject is suffering from recurrent implantation failure (RIF).

In some embodiments, the subject (e.g., an infertile subject) is undergoing an assisted reproductive procedure, or the population of subjects is a population of subjects undergoing an assisted reproductive procedure. In some embodiments, the assisted reproductive procedure is in vitro fertilization. In some embodiments, the subject is undergoing intrauterine insemination or treatments to stimulate egg production, or the population of subjects is a population of subjects undergoing intrauterine insemination or treatments to stimulate egg production.

### Pharmaceutical Composition Comprising a Vaginal Composition

Provided are vaginal compositions, methods of making and using the same, and pharmaceutical compositions comprising the same. The vaginal compositions described herein provide a solution for problems encountered in the past with using isolated and culture-propagated defined or single strain compositions or those containing multiple isolated and culture-propagated strains. Problems include lack of vaginal colonization and engraftment in the urogenital tract, including the vaginal cavity, of the strains that have been administered. The vaginal compositions described herein can be, e.g., administered to the vaginal cavity to modulate the vaginal microbial niche for maintenance of a healthy vaginal microbiota and to help restore an unbalanced vaginal microbiota, e.g., a dysbiotic vaginal microbiota. The administration of a vaginal composition is preferable and advantageous to composition containing single and culture-propagated strains in that it is capable of efficiently colonizing the vaginal microbial niche upon administration to promote embryo implantation in a subject exhibiting RPL, e.g., in a subject undergoing in vitro fertilization (IVF).

The vaginal composition, in embodiments, includes bacteria, viruses, fungi, and microbial metabolites. In embodiments, the vaginal composition further comprises mucus, e.g., vaginal or cervicovaginal mucus, and human donor-derived cells and molecules. In embodiments, one or more of mucus, e.g., cervicovaginal mucus, and human donor-derived cells and molecules is substantially removed. In embodiments, the vaginal composition is isolated, and thus separated, from the host, e.g., a healthy donor subject. The vaginal composition is obtained from a donor subject, e.g., a healthy donor subject, by collecting the mucosal fluid from the genitourinary tract comprising vaginal or cervicovaginal microbes. In embodiments, the vaginal composition further comprises the bacteria, fungi/yeasts and phages including their metabolites and mucus that are found in the genitourinary tract, e.g. the vagina, cervix and/or uterus, of the donor subject.

The urogenital (also known as genital-urinary) tract consists of interconnected biogeographical niches. Bacteria from the vaginal microbial community can migrate through the cervix to remote sites of the urogenital tract. A dysbiosis in the vaginal microbial community can result in a dysbiosis in remote sites, including the uterus. Dysbiosis at these sites has been associated with a range of disease and conditions, including urinary tract infection (UTI), pelvic inflammatory disease (PID), and endometrial receptivity.

In some embodiments, administration of a vaginal composition to the vagina includes resolving dysbiosis in remote sites of the urogenital tract, e.g., in the uterus.

In some embodiments, the vaginal compositions comprise one, two, three, four or five different bacterial species from the genus Lactobacillus. In some embodiments, the vaginal compositions comprise one, two, three, or four different bacterial species from the genus Lactobacillus. In some embodiments, the bacterial species comprise about 80%, about 85%, about 90%, about 91%, about 92%, about 93%, about 94%, about 95%, about 96%, about 97%, about 98%, about 99%, about 99.5%, about 99.9%, 80-99%, 75%-95%, 85%-95%, 85%-99%, or 90%-99% of the preparation (of the total of all detectable bacterial taxa (e.g., species) of the preparation).

In some embodiments, the preparation comprises at least one of (a) to (o) Lactobacillus species and species combinations: a) Lactobacillus crispatus; (b) Lactobacillus iners; (c) Lactobacillus jensenii; (d) Lactobacillus gasseri; (e) Lactobacillus crispatus and Lactobacillus iners; (f) Lactobacillus crispatus and Lactobacillus jensenii; (g) Lactobacillus crispatus and Lactobacillus gasseri; (h) Lactobacillus iners and Lactobacillus jensenii; (i) Lactobacillus iners and Lactobacillus gasseri; (j) Lactobacillus jensenii and Lactobacillus gasseri; (k) Lactobacillus crispatus, Lactobacillus iners and Lactobacillus jensenii; (1) Lactobacillus crispatus, Lactobacillus iners and Lactobacillus gasseri; (m) Lactobacillus crispatus, Lactobacillus jensenii and Lactobacillus gasseri; (n) Lactobacillus iners, Lactobacillus jensenii and Lactobacillus gasseri; (o) Lactobacillus crispatus, Lactobacillus iners, Lactobacillus jensenii and Lactobacillus gasseri.

In one aspect, the vaginal composition (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises less than 5% of Gardnerella spp., Atopobium spp., and Prevotella spp.; wherein the pharmaceutical composition comprises a pharmaceutically acceptable carrier or diluent.

In some embodiments, about 80-99.9% of all detectable bacterial species of the preparation comprise one bacterial species from the genus Lactobacillus, selected from Lactobacillus crispatus, Lactobacillus iners, Lactobacillus jensenii, and Lactobacillus gasseri. In some embodiments, about 80-99.9% of all detectable bacterial species of the preparation comprise Lactobacillus crispatus. In some embodiments, about 80-99.9% of all detectable bacterial species of the preparation comprise Lactobacillus iners. In some embodiments, about 80-99.9% of all detectable bacterial species of the preparation comprise Lactobacillus jensenii. In some embodiments, about 80-99.9% of all detectable bacterial species of the preparation comprise Lactobacillus gasseri.

In some embodiments, about 80-99.9% of all detectable bacterial species of the preparation comprise two bacterial species from the genus Lactobacillus, selected from Lactobacillus crispatus, Lactobacillus iners, Lactobacillus jensenii, and Lactobacillus gasseri. In some embodiments, about 80-99.9% of all detectable bacterial species of the preparation comprise Lactobacillus crispatus and Lactobacillus iners. In some embodiments, about 80-99.9% of all detectable bacterial species of the preparation comprise Lactobacillus crispatus and Lactobacillus jensenii. In some embodiments, about 80-99.9% of all detectable bacterial species of the preparation comprise Lactobacillus crispatus and Lactobacillus gasseri. In some embodiments, about 80-99.9% of all detectable bacterial species of the preparation comprise Lactobacillus iners and Lactobacillus jensenii. In some embodiments, about 80-99.9% of all detectable bacterial species of the preparation comprise Lactobacillus iners and Lactobacillus gasseri.

In some embodiments, about 80-99.9% of all detectable bacterial species of the preparation comprise three bacterial species from the genus Lactobacillus, selected from Lactobacillus crispatus, Lactobacillus iners, Lactobacillus jensenii, and Lactobacillus gasseri.

In some embodiments, about 80-99.9% of all detectable bacterial species of the preparation comprise Lactobacillus crispatus, Lactobacillus iners and jensenii. In some embodiments, about 80-99.9% of all detectable bacterial species of the preparation comprise Lactobacillus crispatus, Lactobacillus iners and gasseri.

In some embodiments, about 80-99.9% of all detectable bacterial species of the preparation comprise four bacterial species from the genus Lactobacillus, selected from Lactobacillus crispatus, Lactobacillus iners, Lactobacillus jensenii, and Lactobacillus gasseri.

In some embodiments, Lactobacillus crispatus or Lactobacillus iners are present in greater relative quantities than other bacterial species of the preparation, for example, 80-99.9% of all detectable bacterial species of the vaginal composition may be Lactobacillus crispatus, further comprising less than 20%, 10%, 5%, 2%, 1%, 0.5% or 0.1% other bacterial species.

### Further Lactobacillus Species

In some embodiments, one or more additional Lactobacillus species are present in the preparations in minor quantities (e.g., less than 20%, 15%, 10%, 5%, 2%, 1% of the Lactobacillus species of the preparation). In some embodiments, these lactobacilli species are present in a concentration of about 0.01-1%, 0.02-0.5% or 0.01-0.3% of all detectable bacterial species of a vaginal composition.

In a further embodiment, the vaginal composition comprises further lactobacilli other than Lactobacillus crispatus, Lactobacillus iners, Lactobacillus jensenii, or Lactobacillus gasseri, wherein the further Lactobacillus species include, e.g., Lactobacillus acidophilus, Limosilactobacillus fermentum (formerly known as Lactobacillus fermentum), Lacticaseibacillus casei (formerly known as Lactobacillus casei), and Lacticaseibacillus rhamnosus (formerly known as Lactobacillus rhamnosus). As used herein, Limosilactobacillus fermentum, Lacticaseibacillus casei, Lacticaseibacillus rhamnosus are referred to as Lactobacillus and are included in the meaning of Lactobacillus.

The vaginal composition comprises less than 5% of Gardnerella spp., Atopobium spp., and Prevotella spp. In some embodiments, vaginal composition of the invention comprises less than 10%, less than 7%, less than 5%, less than 2%, less than 1%, less than 0.5%, or less than 0.1% of Gardnerella spp., Atopobium spp., and Prevotella spp. The preparation can further comprise less than 5% of Gardnerella vaginalis., Atopobium spp., and Prevotella spp. and Fannyhessa vaginae. Gardnerella vaginalis was historically misclassified and has recently been re-classified as belonging to the genus Bifidobacteria. The terms Gardnerella vaginalis and Bifidobacterium vaginalis are thus used interchangeably. Atopobium vaginae and Fannyhessea vaginea are also used interchangeably throughout the application.

Provided herein are vaginal compositions, which are separate from the animal or human body (isolated vaginal compositions). The vaginal composition is thus used interchangeably with isolated vaginal composition.

Lactobacillus crispatus-Dominant Preparations

Provided herein are Lactobacillus crispatus-dominant vaginal compositions, wherein Lactobacillus crispatus is present in a greater amount than each of Lactobacillus iners, Lactobacillus jensenii and Lactobacillus gasseri. Preferred aspects and embodiments of Lactobacillus crispatus-dominant vaginal compositions are provided in the following.

In one aspect, the vaginal composition of the invention comprises Lactobacillus crispatus, in a relative quantity of about 80-99.9% of all detectable bacterial species of the preparation; and less than 5% of Gardnerella spp., Atopobium spp., and Prevotella spp. In some embodiments, preparation comprises Lactobacillus crispatus, in a relative quantity of above 90%, 95%, 99% or 99.9% of all detectable bacterial species of the preparation. In some embodiments, the species Lactobacillus iners, Lactobacillus jensenii, Lactobacillus gasseri are not present in detectable quantities in the preparation.

In another aspect, the vaginal composition of the invention comprises Lactobacillus crispatus and Lactobacillus iners, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri and/or Lactobacillus jensenii. In some embodiments, the preparation comprises Lactobacillus crispatus and Lactobacillus iners which together comprise about 80-99.9% of all detectable bacterial species of the preparation, wherein Lactobacillus iners comprises up to 5%, 10%, 20% or 30% of all detectable bacterial species of the preparation. In some embodiments, the preparation of the invention comprises Lactobacillus crispatus and Lactobacillus iners which together comprise about 80-99.9% of all detectable bacterial species of the preparation, wherein Lactobacillus iners comprises about 5-40%, such as 5-10%, 10-20%, 20-30%, or 30-40% of all detectable bacterial species of the preparation. In some embodiments, the preparation comprises Lactobacillus crispatus and Lactobacillus iners which together comprise about 80-99.9% of all detectable bacterial species of the preparation, wherein Lactobacillus iners comprises at least 5%, 10%, 20% or 30% of all detectable bacterial species of the preparation.

In one aspect, the vaginal composition of the invention comprises Lactobacillus crispatus and Lactobacillus jensenii, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri and/or Lactobacillus iners. In some embodiments, the preparation comprises Lactobacillus crispatus and Lactobacillus jensenii, which together comprise about 80-99.9% of all detectable bacterial species of the preparation, wherein Lactobacillus jensenii comprises at least 0.01 to about 45% of all detectable bacterial species of the preparation. In some embodiments, the preparation of the invention comprises Lactobacillus crispatus and Lactobacillus jensenii, which together comprise about 80-99.9% of all detectable bacterial species of the preparation, wherein Lactobacillus jensenii comprises about 0.01-5%, 5-10%, 10-20%, or 30-40% of all detectable bacterial species of the preparation. In some embodiments, the preparation comprises Lactobacillus crispatus and Lactobacillus jensenii which together comprise about 80-99.9% of all detectable bacterial species of the preparation, wherein Lactobacillus jensenii comprises about 0.01-5% of all detectable bacterial species of the preparation.

In one aspect, the vaginal composition of the invention comprises Lactobacillus crispatus, Lactobacillus iners and Lactobacillus jensenii which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri. In some embodiments, the preparation comprises Lactobacillus crispatus, Lactobacillus iners and Lactobacillus jensenii, which together comprise about 80-99.9% of all detectable bacterial species of the preparation, wherein each of Lactobacillus iners and Lactobacillus jensenii comprises at least 0.01 to about 20% of all detectable bacterial species of the preparation. In some embodiments, the preparation comprises Lactobacillus crispatus, Lactobacillus iners and Lactobacillus jensenii, which together comprise about 80-99.9% of all detectable bacterial species of the preparation, wherein each of Lactobacillus iners and Lactobacillus jensenii comprises at least 0.01 to about 15% of all detectable bacterial species of the preparation. In some embodiments, the preparation comprises Lactobacillus crispatus, Lactobacillus iners and Lactobacillus jensenii, which together comprise about 80-99.9% of all detectable bacterial species of the preparation, wherein each of Lactobacillus iners and Lactobacillus jensenii comprises about 2-15% of all detectable bacterial species of the preparation. In some embodiments, the preparation comprises Lactobacillus crispatus, Lactobacillus iners and Lactobacillus jensenii, which together comprise about 80-99.9% of all detectable bacterial species of the preparation, wherein each of Lactobacillus iners and Lactobacillus jensenii comprises about 2-10% of all detectable bacterial species of the preparation. In some embodiments, the preparation of the invention comprises Lactobacillus crispatus, Lactobacillus iners and Lactobacillus jensenii, which together comprise about 80-99.9% of all detectable bacterial species of the preparation, wherein Lactobacillus jensenii comprises about 0.01-5%, 5-10%, or 10-20% of all detectable bacterial species of the preparation. In some embodiments, the preparation comprises Lactobacillus crispatus Lactobacillus iners and Lactobacillus jensenii, which together comprise about 80-99.9% of all detectable bacterial species of the preparation, wherein Lactobacillus iners comprises about 0.01-5%, 5-10%, or 10-20% of all detectable bacterial species of the preparation. In some embodiments, the preparation comprises Lactobacillus crispatus Lactobacillus iners and Lactobacillus jensenii, which together comprise about 80-99.9% of all detectable bacterial species of the preparation, wherein each of Lactobacillus iners and Lactobacillus jensenii comprises up to 15% of all detectable bacterial species.

In one aspect, the vaginal composition of the invention comprises Lactobacillus crispatus, Lactobacillus iners, Lactobacillus jensenii and Lactobacillus gasseri, which together comprise about 80-99.9% of all detectable bacterial species of the preparation; and less than 5% of Gardnerella spp., Atopobium spp., and Prevotella spp. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein Lactobacillus crispatus comprise at least 50%, 60%, 70% or 80% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein each of Lactobacillus jensenii, Lactobacillus iners and Lactobacillus gasseri comprises 0.01-20%, 0.2-15% or 0.2-10% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein each of Lactobacillus jensenii, Lactobacillus iners and Lactobacillus gasseri comprises 0.01 to about 5% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein Lactobacillus crispatus comprises at least 60% of all detectable bacterial species of the preparation, and further wherein each of Lactobacillus jensenii, Lactobacillus crispatus and Lactobacillus gasseri comprises 0.01 to about 10% of all detectable bacterial species of the preparation.

Lactobacillus iners-Dominant Preparations

The invention further provides Lactobacillus iners-dominant vaginal compositions, wherein Lactobacillus iners is present in a greater amount than each of Lactobacillus crispatus, Lactobacillus jensenii and Lactobacillus gasseri. Preferred aspects and embodiments of Lactobacillus iners-dominant vaginal compositions are provided in the following.

In one aspect, the vaginal composition of the invention comprises Lactobacillus iners, in a relative quantity of about 80-99.9% of all detectable bacterial species of the preparation; and less than 5% of Gardnerella spp., Atopobium spp., and Prevotella spp. In some embodiments, preparation comprises Lactobacillus iners, in a relative quantity of above 90%, 95%, 99% or 99.9% of all detectable bacterial species of the preparation. In some embodiments, the species Lactobacillus crispatus, Lactobacillus jensenii, Lactobacillus gasseri are not present in detectable quantities in the preparation.

In another aspect, the vaginal composition of the invention comprises Lactobacillus iners and Lactobacillus crispatus, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri. In some embodiments, the preparation comprises Lactobacillus iners and Lactobacillus crispatus, which together comprise about 80-99.9% of all detectable bacterial species of the preparation, wherein Lactobacillus crispatus comprises less than 20%, less than 10% or less than 5% of all detectable bacterial species of the preparation. In some embodiments, the preparation of the invention comprises Lactobacillus iners and Lactobacillus crispatus, which together comprise about 80-99.9% of all detectable bacterial species of the preparation, wherein Lactobacillus crispatus comprises 0.01-10%, 0.01-5%, 0.01-2% or 0.01-1%, of all detectable bacterial species of the preparation. In some embodiments, the preparation comprises Lactobacillus iners and Lactobacillus crispatus, which together comprise about 80-99.9% of all detectable bacterial species of the preparation, wherein Lactobacillus iners comprises at least 50%, 60%, 70%, 80%, 90%, 95%, or 99% of all detectable bacterial species of the preparation.

In some embodiments, the preparation comprises Lactobacillus iners and Lactobacillus crispatus, which together comprise about 80-99.9% of all detectable bacterial species of the preparation, wherein Lactobacillus iners comprises at least 70% and Lactobacillus crispatus comprises less than 20% of all detectable bacterial species of the preparation.

In another aspect, the vaginal composition of the invention comprises Lactobacillus iners and Lactobacillus jensenii, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri and/or Lactobacillus crispatus. In some embodiments, the preparation comprises Lactobacillus iners and Lactobacillus jensenii, which together comprise about 80-99.9% of all detectable bacterial species of the preparation, wherein Lactobacillus jensenii comprises less than 1%, 0.5%, 0.2%, 0.1%, 0.05% or 0.01% of all detectable bacterial species of the preparation. In some embodiments, the preparation comprises Lactobacillus iners and Lactobacillus jensenii, which together comprise about 80-99.9% of all detectable bacterial species of the preparation, wherein Lactobacillus jensenii comprises 0.01-0.05% of all detectable bacterial species of the preparation. In some embodiments, the preparation comprises Lactobacillus iners and Lactobacillus jensenii, which together comprise about 80-99.9% of all detectable bacterial species of the preparation, wherein Lactobacillus iners comprises at least 70% and Lactobacillus jensenii comprises less than 1% of all detectable bacterial species of the preparation. In some embodiments, the preparation comprises Lactobacillus iners and Lactobacillus jensenii, which together comprise about 80-99.9% of all detectable bacterial species of the preparation, wherein Lactobacillus iners comprises at least 80% and Lactobacillus jensenii comprises less than 0.1% of all detectable bacterial species of the preparation. In some embodiments, the preparation comprises Lactobacillus iners and Lactobacillus jensenii, which together comprise about 80-99.9% of all detectable bacterial species of the preparation, wherein Lactobacillus iners comprises at least 90% and Lactobacillus jensenii comprises less than 0.1% of all detectable bacterial species of the preparation.

In another aspect, the vaginal composition of the invention comprises Lactobacillus iners and Lactobacillus gasseri, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus jensenii and/or Lactobacillus crispatus. In some embodiments, Lactobacillus jensenii and/or Lactobacillus crispatus comprise less than 2%, 1%, 0.5%, 0.1% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus gasseri comprises less than 5%, 4%, 3%, 2%, or 1% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus gasseri comprises 0.01-5%, 1-4%, or 1-3% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus iners comprises at least 70% and Lactobacillus gasseri comprises less than 5% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus iners comprises at least 80% and Lactobacillus gasseri comprises less than 5% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus iners comprises at least 90% and Lactobacillus gasseri comprises less than 5% of all detectable bacterial species of the preparation.

In one aspect, the vaginal composition of the invention comprises Lactobacillus iners, Lactobacillus crispatus and Lactobacillus jensenii, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri. In some embodiments, the preparation comprises Lactobacillus iners, Lactobacillus crispatus and Lactobacillus jensenii, which together comprise about 80-99.9% of all detectable bacterial species of the preparation, wherein each of Lactobacillus crispatus and Lactobacillus jensenii comprises at least 0.01 to about 10% of all detectable bacterial species of the preparation. In some embodiments, the preparation comprises Lactobacillus crispatus, Lactobacillus crispatus and Lactobacillus jensenii, which together comprise about 80-99.9% of all detectable bacterial species of the preparation, wherein each of Lactobacillus crispatus and Lactobacillus jensenii comprises at least 0.01 to about 5% of all detectable bacterial species of the preparation. In some embodiments, the preparation comprises Lactobacillus iners, Lactobacillus crispatus and Lactobacillus jensenii, which together comprise about 80-99.9% of all detectable bacterial species of the preparation, wherein each of Lactobacillus crispatus and Lactobacillus jensenii comprises about 00.01-1% of all detectable bacterial species of the preparation. In some embodiments, the preparation comprises Lactobacillus iners, Lactobacillus crispatus and Lactobacillus jensenii, which together comprise about 80-99.9% of all detectable bacterial species of the preparation, wherein Lactobacillus jensenii comprises less than 2%, 1%, 0.5%, 0.2% of all detectable bacterial species of the preparation. In some embodiments, the preparation comprises Lactobacillus iners, Lactobacillus crispatus and Lactobacillus jensenii, which together comprise about 80-99.9% of all detectable bacterial species of the preparation, wherein Lactobacillus crispatus comprises less than 2%, 1%, 0.5%, 0.2% of all detectable bacterial species of the preparation. In some embodiments, the preparation comprises Lactobacillus iners, Lactobacillus crispatus and Lactobacillus jensenii, which together comprise about 80-99.9% of all detectable bacterial species of the preparation, wherein each of Lactobacillus iners and Lactobacillus jensenii comprises less than 2%, 1%, 0.5%, 0.2% of all detectable bacterial species in the preparation.

In one aspect, the vaginal composition of the invention comprises Lactobacillus iners, Lactobacillus crispatus and Lactobacillus gasseri, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus jensenii. In some embodiments, the preparation comprises Lactobacillus iners, Lactobacillus crispatus and Lactobacillus gasseri, which together comprise about 80-99.9% of all detectable bacterial species of the preparation, wherein each of Lactobacillus crispatus and Lactobacillus gasseri comprises at least 0.01 to about 10% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein each of Lactobacillus crispatus and Lactobacillus gasseri comprises at least 0.01 to about 5% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein each of Lactobacillus crispatus and Lactobacillus gasseri comprises about 00.01-1% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein Lactobacillus gasseri comprises less than 2%, 1%, 0.5%, 0.2% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein Lactobacillus crispatus comprises less than 2%, 1%, 0.5%, 0.2% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein each of Lactobacillus iners and Lactobacillus gasseri comprises less than 2%, 1%, 0.5%, 0.2% of all detectable bacterial species in the preparation.

In one aspect, the vaginal composition of the invention comprises Lactobacillus crispatus, Lactobacillus iners, Lactobacillus jensenii and Lactobacillus gasseri, which together comprise about 80-99.9% of all detectable bacterial species of the preparation; and less than 5% of Gardnerella spp., Atopobium spp., and Prevotella spp. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein Lactobacillus iners comprises at least 50%, 60%, 70% or 80% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein each of Lactobacillus jensenii, Lactobacillus iners and Lactobacillus gasseri comprises 0.01-25%, 0.1-20% or 0.2-15% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein each of Lactobacillus jensenii, Lactobacillus crispatus and Lactobacillus gasseri comprises 0.01 to about 5% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein Lactobacillus iners comprises at least 50% of all detectable bacterial species of the preparation, and further wherein each of Lactobacillus jensenii, Lactobacillus crispatus and Lactobacillus gasseri comprises 0.01 to about 25% of all detectable bacterial species of the preparation.

Lactobacillus jensenii-Dominant Preparations

The invention further provides Lactobacillus jensenii-dominant vaginal compositions, wherein Lactobacillus jensenii is present in a greater amount than each of Lactobacillus crispatus, Lactobacillus iners and Lactobacillus gasseri. Preferred aspects and embodiments of Lactobacillus jensenii-dominant vaginal compositions are provided in the following.

In one aspect, the vaginal composition of the invention comprises Lactobacillus jensenii, Lactobacillus crispatus, Lactobacillus iners and Lactobacillus gasseri in a relative quantity of about 80-99.9% of all detectable bacterial species of the preparation; and less than 5% of Gardnerella spp., Atopobium spp., and Prevotella spp. In some embodiments, preparation comprises Lactobacillus jensenii in a relative quantity of above 60%, 65%, 70% or 75% of all detectable bacterial species of the preparation. In some embodiments, each of Lactobacillus crispatus, Lactobacillus iners, Lactobacillus gasseri comprises 0.01 to about 20% of all detectable bacterial species of the preparation. In some embodiments, each of Lactobacillus crispatus, Lactobacillus iners, Lactobacillus gasseri comprises 0.05 to about 15% of all detectable bacterial species of the preparation. In some embodiments, Lactobacillus jensenii comprises at least 65% of all detectable bacterial species of the preparation, and further wherein each of Lactobacillus crispatus, Lactobacillus iners, Lactobacillus gasseri comprises 0.01 to about 15% of all detectable bacterial species of the preparation.

Lactobacillus gasseri-Dominant Preparations

The invention further provides Lactobacillus gasseri-dominant vaginal compositions, wherein Lactobacillus gasseri is present in a greater amount than each of Lactobacillus crispatus, Lactobacillus jensenii and Lactobacillus gasseri. Preferred aspects and embodiments of Lactobacillus gasseri-dominant vaginal compositions are provided in the following.

In one aspect, the vaginal composition of the invention comprises Lactobacillus gasseri, in a relative quantity of about 80-99.9% of all detectable bacterial species of the vaginal composition; and less than 5% of Gardnerella spp., Atopobium spp., and Prevotella spp. In some embodiments, the vaginal composition comprises Lactobacillus gasseri, in a relative quantity of above 90%, 95%, 99% or 99.9% of all detectable bacterial species of the vaginal composition. In some embodiments, the species Lactobacillus crispatus, Lactobacillus jensenii, Lactobacillus gasseri are not present in detectable quantities in the vaginal composition.

In another aspect, the vaginal composition of the invention comprises Lactobacillus gasseri, Lactobacillus crispatus, Lactobacillus iners and or Lactobacillus jensenii, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus iners and or Lactobacillus jensenii. In some embodiments, the preparation does not comprise detectable quantities of Lactobacillus crispatus and or Lactobacillus jensenii. In some embodiments, the preparation does not comprise detectable quantities of Lactobacillus iners and or Lactobacillus crispatus. In some embodiments, the preparation comprises Lactobacillus gasseri in a relative quantity of at least 40%, 45%, 50%, 55%, 60%, 65% or more of all detectable bacterial species of the preparation. The vaginal composition, and pharmaceutical composition comprising the same, are suitable for administration, preferably vaginal administration, to a subject. The subject can be a human. In one aspect, the vaginal composition provided herein is suitable for vaginal administration. In a further aspect, the vaginal composition provided herein is for use in treating inflammation or a disorder associated with inflammation. In a further aspect, the vaginal composition provided herein comprises an effective amount of vaginal lactobacilli for engrafting in a recipient's vaginal niche and treating inflammation.

### Lactobacilli in the Vaginal Niche

Lactobacilli are the predominant microorganisms in the healthy vaginal microbial community and they play a major role in maintaining a healthy urogenital tract. Lactobacilli are capable of preventing adhesion and growth of pathogenic microorganisms and/or overgrowth of pathobionts through mechanisms that appear to involve secretion of anti-adhesion factors, hydrogen peroxide, bacteriocins and fermenting the glycogen to lactic acid, thereby creating an acidic environment hostile to pathogens and pathobionts. The genus Lactobacillus comprises a phenotypically heterogenous group of Gram-positive, aerotolerant anaerobic, lactic acid producing bacteria. Other typical characteristics include being catalase-negative and rod-shaped, and generally possess DNA with a low content of guanine (G) and cytosine (C), less than about 50%. They are members of the phylum Firmicutes, class Bacilli, order Lactobacillales and family Lactobacillaceae. One skilled in the art will be able to identify Lactobacillus species using standard techniques.

Species of lactobacilli can be identified phenotypically, as well as genetically, e.g., on the basis of 16S rRNA (ribosomal RNA) sequence (or the DNA encoding the 16S rRNA, generally referred to as 16S rDNA). Genetic analysis can be performed using standard techniques, for example whole genome sequencing analysis as well as widely used typing approaches based on nucleotide variation in several hundred DNA sequences and a few gene fragments: Multi-locus Sequence Typing (MLST), Multi-locus Variable number of tandem repeats Analysis (MLVA), rMLST and cgMLST) discussed, e.g., in Marcos Perez-Losada, M. et al., “Microbial sequence typing in the genomic era”, Infection, Genetics and Evolution, Vol. 63, September 2018, p. 346-359. Other identification techniques include: Vaginal pH, Nugent Score, Whiff Test, gas liquid chromatographic analysis of glucose fermentation products, total anaerobe concentrations, total aerobe concentrations, enzymatic activity (e.g., lipase, phospholipase A2 and phospholipase C, hydrogen peroxide production).

In some embodiments, the vaginal compositions described herein do not comprise (and are not derived from) isolated and/or culture-propagated bacterial strain(s). vaginal compositions can be prepared from cervicovaginal secretions (vaginal fluid), e.g., collected from the vaginal tract of a donor with a healthy vaginal flora. vaginal compositions can be collected using standard techniques using commercially available collection devices, such as, a menstrual fluid collection device (soft cup or soft disc), a syringe, a tube, spatula or beaker, or an absorbent matrix. In some embodiments, the menstrual fluid collection device is a vaginal self-sampling device. A vaginal self-sampling device can be, e.g., used by donors to collect vaginal fluid or cervicovaginal secretions without the help of another person.

Optionally, the collected material is undergoing centrifugation. The centrifugation step may be performed to facilitate collection, without physical separation of vaginal fluid components. In some embodiments, the vaginal compositions comprise one or more of: mucus (e.g., secreted by the cervix), shed epithelial cells, vaginal transudate, and bacteria other than lactobacilli found in the secretion from the donor. It is thought that mucus and other components of the secretion (including other bacteria) are beneficial to Lactobacillus growth and survival upon administration to the urogenital tract thereby supporting engraftment in a recipient. This provides an advantage over compositions comprising isolated strain(s). Thus, in some embodiments, the vaginal composition is not cultured (e.g., for the purpose of strain isolation) or propagated in vitro but rather preferably stored in the refrigerator at 4° C. or immediately frozen after collection (e.g., frozen to 0° C., −20° C., −80° C., −190° C.), or optionally spray dried or lyophilized.

If desired, the cervicovaginal secretions can be further processed, e.g., prior to refrigeration or freezing, e.g., by filtration for sterility and/or to remove residual particles, aggregates and cells, and adding diluent, e.g., to arrive at a desired volume, concentration (e.g., CFU/mL) and/or viscosity, as discussed herein. Optionally, the vaginal composition is kept refrigerated or frozen until it is formulated into a dosage form and/or dispensed into an applicator or dispenser.

Other advantages of the vaginal compositions described herein include that, in embodiments, substantial engraftment does not require the use of mucus adhesive excipients (such as, e.g., hydrocolloids, e.g., xanthan gum, locust bean gum alginate) to increase the ability to colonize by adherence of lactobacilli to the mucosal membrane, which is sometimes used to increase engraftment of isolated, propagated strains. This minimizes the risk of triggering an adverse side effect, such as an allergic reaction due to the mucus adhesive excipient and increases tolerance.

Another advantage of the vaginal compositions described herein is the efficient and prolonged engrafting/colonization of the lactobacilli in the vaginal mucosa. In some embodiments, the colonization effect persists for at least 1, 2, 3, 4, 5 or more menstrual cycles after administration of the vaginal composition.

### Some Composition Characteristics

Provided herein are compositions, such as pharmaceutical compositions comprising the vaginal compositions. In some embodiments, the pharmaceutical compositions comprise i) a vaginal composition comprising, e.g., one or more Lactobacillus species and optionally one or more residual constituents of a cervicovaginal secretion (such as, e.g., vaginal mucus, vaginal transudate, other vaginal fluids, and vaginal epithelial cells), ii) a pharmaceutically acceptable buffer or diluent or other excipients, such as, e.g., saline (e.g., to adjust the viscosity and/or isotonicity/osmolarity of the composition), iii) one or more acidifying agents, e.g., lactic acid or boric acid (e.g., to adjust the pH of the composition, e.g., to below pH 5, or between pH 3.0 and 4.5), iv) one or more other active agents, such as, e.g., prebiotics (e.g., to generate a symbiotic mixture with the vaginal composition), spermicides (e.g., to kill or inactivate residual sperm in the preparation); hormonal agents (e.g., estrogen), anti-inflammatory agents, and the like. In preferred embodiments, the pharmaceutical compositions comprise (i) and (ii). In one embodiment, the pharmaceutical compositions consist of (i) and (ii). In one embodiment, the pharmaceutical compositions comprise (i) and (ii) but not (iii) and (iv). In one embodiment, the pharmaceutical compositions comprise (i), (ii), and (iii) but not (iv). In one embodiment, the pharmaceutical compositions comprise (i), (ii), (iii) and a spermicide. In some embodiment, the pharmaceutical compositions do not comprise prebiotics, hormonal or anti-inflammatory agents.

### Adjuvant, Excipient and Other Agents

The compositions comprising a vaginal composition described herein may optionally comprise one or more adjuvants, excipient or agents, including but not limited to acidifying agents, pharmaceutically acceptable buffers or diluents (e.g., saline (e.g., 0.9% NaCl)), spermicides, prebiotics or other agents.

### Acidifying Agents

Acidifying agents may comprise, e.g., organic acids or salts thereof. In some embodiments, acidifying agents may be used to reduce the pH of the compositions, e.g., to below pH 5.5 or 5.0, e.g., to between pH 3.5 to 4.5, or pH 3.0 to 4.5. In decreasing pH such acidifiers may act as anti-microbial agents (e.g., to inhibit Candida or pathogenic bacteria). Acidifying agents comprise, e.g., lactic, acetic, ascorbic, citric, folic sorbic, or boric acid. In other embodiments, the acidifying agent is administered separate from the compositions comprising a vaginal composition; e.g. prior to, concurrent with, or after administration of the composition (e.g., in a different dosage form, such as, e.g. a suppository, cream, gel, powder, douche or similar), e.g., for one to seven days, or one to ten days. In these embodiments, the acidifying agent may be used to promote the survival and engraftment of the lactobacilli comprised in the vaginal composition. Acidifying agents may also be useful as spermicides, e.g., to kill or inactivate residual sperm that may be present in the preparation. In one embodiment, the spermicidal activity is contributed by adding lactic acid. In one embodiment, lactic acid may be provided as a racemic mixture of D- and L-isomers, or at different suitable ratio, including, e.g., only D-lactate or only L-lactate.

In some embodiments, the compositions comprising a vaginal composition is acidified by adding 0.1%, 0.5%, 1%, 1.5%, 2%, 2.5%, 3% or 5% of acidifier (e.g., lactic acid). In preferred embodiments, the acidifier is added at a concentration of 0.5% to 1.5%, or 0.2% to 2% (w/w), or a similar concentration that matches a healthy vagina.

### Viscosity

The compositions comprising a vaginal composition described herein may comprise one or more pharmaceutically acceptable buffer or diluent or other excipients. In preferred embodiments, the diluent is saline (e.g., 0.9% NaCl), e.g., sterile normal saline. In one embodiment, the diluent is used to adjust the viscosity of the composition, e.g., to lower the viscosity of the vaginal composition derived from the cervicovaginal secretion, thereby increasing the ability to administer the composition to a recipient, e.g., using an applicator or dispenser or similar vaginal delivery system. In other embodiments, one or more excipients is used to formulate the composition in different dosage forms, such as, e.g., suppositories, creams or dissolving films or tablets.

In some embodiments, the dosage form is liquid, solid or semi-solid. The solid dosage form preferably comprises a tablet, capsule, or a film. The semi-solid dosage form preferably comprises a suppository, ointment, gel, cream or rigid foam. In a preferred embodiment, the dosage form is a gel.

When adjusting the viscosity for administration to a recipient's vaginal cavity, e.g., using an applicator or dispenser, care should be taken to adjust the viscosity to be easily dispensable in the vaginal cavity (e.g., without having to apply significant force to expel the liquid composition), yet avoid making the composition so fluid/liquid that is does not stay in the vaginal cavity for any appreciable amount of time after administration. Thus, a viscosity should be selected that is suitable to prevent rapid discharge from the vaginal cavity. The compositions comprising a vaginal composition described herein are preferably of a viscosity which allows the majority of it to stay in the vagina for at least 5 minutes, at least 10 minutes, at least 20 minutes, at least 30 minutes, when the recipient is in an upright position, although administration is preferably carried out in a lithotomy position, e.g., with the recipient lying down. Viscosity can be measured, e.g., using a Viscometer.

### Spermicides

The compositions comprising a vaginal composition described herein may comprise one or more spermicides. Care should be taken when using nonoxynol-9 (N-9) because of its membrane disruptive properties (it increases the permeability of vaginal tissue by causing damage the cervicovaginal epithelium) and has been shown to be detrimental to Lactobacillus spp. Other spermicides include octoxynol-9, sodium cholate, and benzalkonium chloride. In one embodiment, lactic acid is used. In other embodiments, lactic acid may be used in combination, e.g., with citric acid.

### Prebiotics

A “prebiotic” as used herein is a growth substrate, which increases growth of bacteria (such as lactobacilli) comprised in the vaginal compositions, as could be measured, e.g., in vitro. If desired, though it is not generally necessary, a prebiotic may be added to the compositions described herein, e.g., to create a synbiotic mixture, e.g., to increase growth of the bacteria comprised in the vaginal compositions upon administration to the genitourinary tract of a recipient. This may, under certain circumstances, increase successful colonization and engraftment. In other embodiments, prebiotics may be used for the maintenance of an engrafted preparation and/or general vaginal health.

If a prebiotic is desired, it should be carefully chosen to not be greatly metabolizable by any yeast (e.g., Candida species), pathobionts or pathogens (e.g., E. coli and other Gram-negative bacteria) that may reside in the recipient's genitourinary tract (e.g., to avoid promoting their growth). Prebiotics include, e.g., lactitol, lactulose, and in some instances also other oligosaccharides and soluble fibers, e.g., fructo-oligosaccharides (FOS), gluco-oligosaccharides (GOS), and inulin.

### Other Active Agents

If desired, other active agents may be added to the compositions described herein, e.g., to address bacterial or fungal infections and/or sexually transmitted diseases in the recipient, for example antimicrobial agents, antifungal agents, antibacterial agents, antiviral agents, antibiotics, antiparasitic agents (e.g., with activities against Trichomonas vaginalis), anti-inflammatory agents, and the like. Care must be taken when formulating these agents into the pharmaceutical composition so as to not substantially interfere with the activity and efficacy of the vaginal compositions (and lactobacilli) comprised in the compositions.

In some embodiments, the pharmaceutical compositions comprise a form of estrogen. Adequate levels of estrogens play a role in the trophism of vaginal mucosa, and estrogens increase the cellular content of glycogen.

In some embodiments, the pharmaceutical compositions comprise thiosulfate, e.g., to potentiate the anti-pathogenic effect of lactobacilli.

If desired, the pharmaceutical compositions can further contain an antibiotic, such as, e.g., metronidazole, or one or more antibiotics of the following classes: a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a beta-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), and the like.

In some embodiments, the pharmaceutical compositions can further contain an antimicrobial (an antibiotic or antifungal) selected from metronidazole, tinidazole, secnidazole, clindamycin, nystatin, azithromycin, erythromycin, ofloxacin, doxycycline, levofloxacin, amoxicillin, and fluconazole.

If desired, the pharmaceutical composition can contain an agent for treating infections with mycobacteria. Suitable agents for treating infections with mycobacteria include an aminoglycoside (e.g., capreomycin, kanamycin, streptomycin), a fluoroquinolone (e.g. ciprofloxacin, levofloxacin, moxifloxacin), isozianid and isozianid analogs (e.g. ethionamide), aminosalicylate, cycloserine, diarylquinoline, ethambutol, pyrazinamide, protionamide, rifampin, and the like.

If desired, the pharmaceutical composition can contain a suitable antiviral agent, such as remdesivir, oseltamivir, zanamavir, amantidine or rimantadine, ribavirin, gancyclovir, valgancyclovir, foscavir, Cytogam® (cytomegalovirus immune globulin), pleconaril, rupintrivir, palivizumab, motavizumab, cytarabine, docosanol, denotivir, cidofovir, and acyclovir.

If desired, the pharmaceutical composition can contain a suitable antifungal agent, such as polyene (e.g., nystatin and natamycin) and imidazole antifungals (e.g., flucanozole and clotrimazole).

If desired, the pharmaceutical composition can contain one or more suitable steroids. For example, the composition may include androgens/anabolic steroids, estrogens, progestogens, corticosteroids, neurosteroids, estradiol, estropipate, premarin, drospirenone, noresthisterone, levonorgestrel, testosterone, fluoxymesterone, methylesterosterone, oxandrolone, and oxymetholone.

### Combination Therapies

Provided herein are methods to treat a subject, e.g., a human, in need thereof, e.g., a subject exhibiting dysbiosis (e.g., dysbiosis in the urogenital tract and being sub-fertile or infertile), by administering an effective amount of a composition, such as a pharmaceutical composition comprising a vaginal composition described herein.

It will be understood by one of skill in the art that the administration regimen for treating may include one or more other therapies in combination with the administration of the vaginal compositions and compositions described herein. In some embodiments, the methods for treating vaginal dysbiosis described herein may further comprise administering standard of care treatment. For example, the methods for treating vaginal dysbiosis described herein may further comprise administering antimicrobial agents, antifungal agents, antibacterial agents, antiviral agents, antibiotics, antiparasitic agents (e.g., with activities against Trichomonas vaginalis), anti-inflammatory agents, and the like. Current standard of care antimicrobials (antibiotics, antifungals) include metronidazole, tinidazole, secnidazole, clindamycin, nystatin, azithromycin, erythromycin, ofloxacin, doxycycline, levofloxacin, amoxicillin, and fluconazole. In another example, the methods for treating vaginal dysbiosis described herein may further comprise administering thiosulfate, e.g., to help recolonize the vaginal microbiota and to prevent the regrowth of pathogenic agents and thus recurrences.

Any of the therapeutic agents described herein can be considered a) for formulation as a pharmaceutical composition with the vaginal compositions, or b) as a combination therapy.

These combination therapies may be administered together or separately from and concurrent with (substantially the same time) or sequentially to (e.g., prior to or after) administration of the vaginal compositions and compositions described herein. They may be administered using different routes of administration and dosage forms, such as orally (e.g., as a pill) or topical (e.g., as a gel). The use combination therapies should be assessed to determine that the treatment does not substantially interfere with the activity and efficacy of the vaginal compositions (and lactobacilli) comprised in the compositions and if they do, the regimen should be adjusted (e.g., timing, dosing, sequencing, etc.) to minimize the interference.

### Dosage Forms and Formulations

Multiple dosage forms comprising a vaginal composition suitable for the vaginal delivery are contemplated herein, and include a suspension, spray, gel, cream, ointment, powder, (gelatin or vegetable cellulose) capsule, solution for lavages or douches, foams, films, ovules, a vaginal insert (e.g. tampon), tablets, disk, wafer (e.g., drying on film, by vaporization), or a microencapsulated product employing excipients and formulation techniques known to those skilled in the art. Particularly preferred dosage forms include formed gels, lyophilized gels, tablets, frozen formulations and films.

A number of suitable excipients can be used to formulate the vaginal composition, such as bulking agents, polymers, carbon sources, mucoadhesive agents, or pH modifiers and/or buffers.

The carbon source excipients may act as a carbon source for the microbiota contained in the vaginal composition. Such carbon courses comprise mannitol, maltodextran, and Guar gum.

Some excipients may further serve as mucoadhesive agents or as viscosity agents. Bulking agents may comprise one or more of mannitol, micro-crystalline cellulose, maltodextran, guar gum, inulin, or alginic acid (e.g., sodium alginate). Polymers may comprise structural polymers.

In some embodiments, polymers comprise one or more of mucin, hyaluronic acid, polyvinyl alcohol, sodium CMC, polyvinylpyrrolidone, hydroxypropyl methylcellulose, carbopol (e.g., Carbopol 934), and poloxamer (e.g., poloxamer 407). Mucoadhesive agents comprise but are not limited to alginic acid (sodium alginate) and sodium CMC. Viscosity agents comprise but are not limited to Guar gum and Carbopol 934.

Some excipients serve as pH modifiers and/or buffers, such as lactic acid and acetate buffer.

Suitable formulations show little to no flow on suitable vertical surfaces and maintain high bacterial viability (e.g., CFU count) both upon formulation and during (long-term) storage.

Desired formulation selection parameters include, for example, mucoadhesion (of reconstituted product, e.g., in the vaginal tract); viscosity (of reconstituted product), e.g., final viscosity for gel-based product needs to be syringeable at ambient temperature and preferably congealed at 37° C. (at body temperature, e.g., in the vaginal tract); total sugar content (of reconstituted product), e.g., ideally at or lower than physiological concentration (about 0.5-1.0 mg/mL); volume of reconstituted product, e.g., up to 3 mL; hydration rate/disintegration rate (e.g., of gel/matrix), e.g., sufficient physical integrity to provide desired release rate; pH, e.g., between about pH 3.4-3.9 (e.g., to promote inhibition of competitive vaginal bacteria); water activity/moisture content (e.g., of dried formulations), e.g., between 0.5-3% water (e.g., for longer term dried formulation stability); microbial diversity, e.g., relative abundance of Lactobacillus species, such as, e.g., L. crispatus, L. gasseri, L. jensenii, and L. iners; total dose/potency, e.g., preferably at least about 1×10⁵ CFU/VCC, at least about 10⁶ CFU/VCC, at least about 10⁷ CFU/VCC or at least about 10⁸ CFU/VCC per administration (per dose), shelf-life (not reconstituted) at various temperatures, and microbial limits, e.g., absence of microorganisms such as, Pseudomonas aeruginosa, Candida albicans, Staphylococcus aureus, Ph Eur criteria 5.1.4, 2.6.12 & 2.6.13).

Suitable testing methods include standard assays, such as, plate count (e.g., MRS agar), e.g., for life bacteria count, dose determination, shelf-life; rheometer, e.g., for mucoadhesion and viscosity, pH meter, Karl Fisher/water activity meter, Ph Eur testing, e.g., for microbial loads. vaginal compositions can be lyophilized and formulated into, e.g., gels and tablets as well as other dosage forms that can be filled with lyophilized products, such as, e.g., capsules. Vaginal compositions can also be formulated into gels that can be frozen, as well as into liquid media (e.g., with glycerol) that can be frozen. Vaginal composition can also be formulated into (air-dried) films, that could be, e.g., shaped like disks. Losses in viability range from approximately 0.5 log to 1 log at the formulation step depending on excipient and dosage form.

In preferred embodiments, the vaginal composition of the invention is provided in a dosage form selected from a tablet, pre-formed gel, lyophilized gel, liquid formulation, frozen formulation, film-forming formulation or film.

### Pre-Formed and Frozen Gels

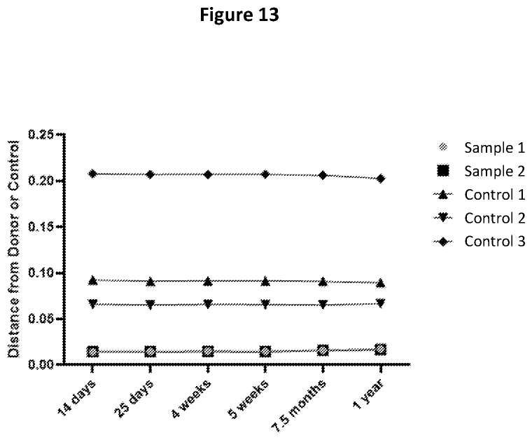

The vaginal composition of the invention can be comprised in a pre-formed gel. In some embodiments, the pre-formed gel is provided in a vial, e.g., for drawing up into a syringe. In some embodiments, the pre-formed gel is provided in a suitable applicator, e.g., as shown in FIG. 13. The pre-formed gel may be stored frozen or refrigerated to maintain the stability of the vaginal composition. In some embodiments, the pre-formed gel comprises hyaluronic acid. In some embodiments, hyaluronic acid is comprised in a concentration of about 0.3-3%. In some embodiments, hyaluronic acid is comprised in a concentration of about 0.5-2%. In some embodiments, the pre-formed gel comprising hyaluronic acid may be frozen at −80° C. In some embodiments, the frozen pre-formed gel comprising hyaluronic acid is substantially stable for a time period of at least 3 months, 6 months, 9 months, 12 months, 18 months or longer.

### Lyophilized Gels

The vaginal composition of the invention can be comprised in in a lyophilized gel. The lyophilized gel may is stable and may be stored for extended periods of time at 2-8° C. The lyophilized gel comprises the lyophilized vaginal composition and a gel forming excipient. The lyophilized gel may be supplied in a vial. The lyophilized gel can be reconstituted prior to administration (e.g., in a clinical or home setting) with a reconstitution agent. In some embodiments, the reconstitution agent comprises a gel, a gel forming agent, or a liquid. The liquid may comprise water, saline or another liquid suitable for reconstitution and subsequent administration to a subject.

In particular embodiments, the lyophilized gel comprising the vaginal composition of the invention further comprises sodium-carboxymethylcellulose (Na-CMC). In some embodiments, Na-CMC is comprised in a concentration of about 1-3%. In some embodiments, Na-CMC is comprised in a concentration of about 2%. In particular embodiments, the lyophilized gel comprises the vaginal composition of the invention and hyaluronic acid. In some embodiments, hyaluronic acid is comprised in a concentration of about 0.3-3%. In some embodiments, hyaluronic acid is comprised in a concentration of about 0.5-2%. In particular embodiments, the lyophilized gel comprises the vaginal composition of the invention and hyaluronic acid and sodium-carboxymethylcellulose (Na-CMC) at the above concentrations. The lyophilized gel comprising NA-CMP, hyaluronic acid, or a combination of both, is stable at 2-8° C. or at −20° C. for a time period of at least 3 months, 6 months, 9 months, 12 months, 18 months or longer. In some embodiments, the lyophilized gel comprising hyaluronic acid may be frozen at −80° C. In some embodiments, the frozen lyophilized gel comprising hyaluronic acid is stable for a time period of at least 3 months, 6 months, 9 months, 12 months, 18 months or longer. In some embodiments, the lyophilized gel and the reconstitution agent are provided as a kit.

### Film-Forming Formulations

The vaginal composition of the invention can be comprised in in a film-forming formulation. The vaginal composition of the invention can be formulated with polymeric excipients, wherein the polymeric excipients have bioadhesive properties and film-forming capacity. Exemplary polymeric excipients for film-forming formulations include polyvinyl alcohol (PVA), sodium lactate and lactic acid. In particular embodiments, the film-forming formulation comprises polyvinyl alcohol (PVA). In some embodiments, the PVA is comprised in a concentration of about 10-25%, 10-20%, or about 12-15%. In particular embodiments, the film-forming formulation comprises PVA in a concentration of about 12%. In some embodiment, the film-forming formulation comprises PVA, e.g., in a concentration of about 10-25%, 10-20%, 1²-15% or 12%, and is air-dried. In some embodiments, the film-forming formulation is provided as a disc or wafer. In some embodiments, the film-forming formulation is provided as a mucoadhesive pessary or patch. The film-forming formulation is substantially stable for a time period of at least 3 months, 6 months, 9 months, 12 months, 18 months or longer at 2-8° C. In some embodiments, the film-forming formulation rapidly disperses or dissolves in contact with fluids, e.g., cervicovaginal solution, to intravaginally form a viscous and bioadhesive gel. In some embodiments, formation of a bioadhesive dispersion is retained in the vagina for prolonged periods of time.

### Tablets

The vaginal composition of the invention can be comprised in in a tablet. The tablet may comprise agents with gel-forming properties, muco-adhesive properties, or a combination of both. The tablet may further comprise excipient, such as swelling agents, bulking agents, lactic acid, carbopol, HPMC, alginate, or sodium-carboxymethylcellulose (Na-CMC). In some embodiments, the bulking agent comprises microcrystalline cellulose, HPMC/PVP, maltodextran, or poloxamer 407. In particular embodiments, the tablet comprises the vaginal composition of the invention and Na-CMC. In particular embodiments, the tablet comprises the vaginal composition of the invention and polyvinylpyrrolidone (PVP). The tablet may further be substantially stable, e.g., substantially retain Lactobacilli viability. The tablet may be substantially stable at 2-8° C. or at room temperature (about 25° C.) for a time period of at least 3 months, 6 months, 9 months, 12 months, 18 months or longer. In particular embodiments, the tablet comprises the vaginal composition of the invention and sodium-carboxymethylcellulose (Na-CMC) and is stable at 2-8° C. or at room temperature (about 25° C.) for a time period of at least 3 months, 6 months, 9 months, 12 months, 18 months or longer. In particular embodiments, the tablet comprises the vaginal composition of the invention and polyvinylpyrrolidone (PVP) and is stable at 2-8° C. or at room temperature (about 25° C.) for a time period of at least 3 months, 6 months, 9 months, 12 months, 18 months or longer.

### Liquid Formulations

The vaginal composition of the invention can be comprised in in a liquid formulation. In some embodiments, the liquid formulation may be provided as a frozen formulation. In some embodiments, the liquid formulation is provided with gelling agents in a kit. The liquid formulation may comprise a cryoprotectant, such as glycerol, wherein the glycerol concentration is optionally less than 25%, less than 20%, less than 15%, or less than 10%. In some embodiments, the liquid formulation may be frozen, e.g., at −20° C. or −80° C., and substantially retains Lactobacillus viability. In some embodiments, the liquid formulation further comprises lactic acid. In some embodiments, the liquid formulation comprises lactic acid in a concentration of about 0.5 to 2%, preferably in a concentration of about 1-1.5%. In a preferred embodiment, the liquid formulation comprises lactic acid and a cryoprotectant, such as glycerol. In some embodiments, the frozen liquid formulation comprising the cryoprotectant, and optionally lactic acid, is substantially stable for a time period of at least 3 months, 6 months, 9 months, 12 months, 18 months or longer. In some embodiments, the liquid formulation comprising the vaginal composition is provided with a gelling agent as a kit. In some embodiments, a frozen liquid formulation comprising the vaginal composition is provided with a gelling agent as a kit. In some embodiments, a frozen liquid formulation which does not comprise the vaginal composition is provided with a lyophilized gel as a kit, wherein the lyophilized gel comprises the vaginal composition.

In some embodiments, the liquid formulation (or composition) comprising the vaginal composition further comprises saline or phosphate-buffered saline (PBS). In some embodiments, the liquid formulation comprises a ratio of vaginal composition relative to saline or PBS that is in a range of 1:10 to 10:1. In some embodiments, the ratio of vaginal composition relative to saline or PBS is about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10.

In some embodiments, a liquid formulation is frozen and stored at cold temperatures (e.g., below 0 degrees Celsius, below −20 degrees Celsius, or at about −80 degrees Celsius) for a period of time prior to administration to a subject. In some embodiments, the liquid formulation undergoes one or more freeze-thaw cycles. In some embodiments, the liquid formulation is frozen and then subsequently thawed prior to administration via an insemination catheter to a subject. In some embodiments, the liquid formulation is frozen and stored in a prefilled syringe.

### Methods of Producing Preparations and Compositions and Donor Considerations

Aspects of the invention include methods of producing a vaginal composition as described herein. These methods include selecting a suitable donor to provide samples of vaginal fluids, preferably a cervicovaginal secretion, and processing the samples to provide compositions (such as pharmaceutical compositions) comprising the vaginal composition described herein.

In one aspect, the method of producing a vaginal composition, as described herein comprises
d. Processing a microbiota sample from a donor's genitourinary tract, e.g., a vaginal mucosal sample or cervicovaginal secretion, comprising urogenital microbes and vaginal mucosal fluid from the vaginal cavity of a healthy donor subject in a centralized processing facility,
e. Assessing the absence of one or more pathogens,
f. Assessing viability and/or quantity of the urogenital microbes, and
g. Releasing the composition comprising the processed vaginal mucosal sample from quarantine for use in RPL treatment (e.g., IVF procedure), if a predetermined level is obtained in step (b) and (c).

In embodiments, the method may further comprise one, two, three, four or all of the following steps:
n. Adding at least one pharmaceutically acceptable diluent, excipient or carrier;
o. Adjusting the pH, osmolarity and/or viscosity of the vaginal mucosal fluid;
p. Adding one or more cryoprotectants (e.g., for freezing) and/or one or more lyoprotectants (e.g., for drying);
q. Formulating the processed mucosal fluid into a dosage form comprising a powder, a solid, a semi-solid, or a liquid:
r. Partitioning the vaginal mucosal fluid into discrete units, each unit comprising an effective dose of urogenital microbes, wherein the effective dose of urogenital microbes comprises at least 10⁵ colony forming units (CFU), preferably at least 10⁶ colony forming units (CFU), more preferably at least 10⁷ colony forming units (CFU);
s. Storing the refrigerated, frozen or dried vaginal mucosal fluid sample or processed preparation under quarantine;
t. Holding the refrigerated, frozen or dried vaginal mucosal fluid sample or processed preparation under quarantine until any completion of any combination of (a) testing the donor to exclude the substantial presence of one or more transmissible pathogens, (b) confirming the composition and viability of the urogenital microbes comprised, or (c) further confirming the health of the donor by a plurality of post-screening tests;
u. Standardizing the cell count and/or the quantity or concentration of the urogenital microbes comprised in the preparation, optionally by adding an inert filler; and
v. Releasing the refrigerated, frozen or dried mucosal fluid sample or processed preparation from quarantine to define the vaginal composition.

In embodiments for the methods provided herein, one or more (pharmaceutically acceptable) excipients or carriers are added to the mucosal fluid or the mucosal fluid is otherwise further processed. In embodiments, one or more of: pH, osmolarity and/or viscosity are adjusted, e.g., by adding acids, bases, buffers, and/or salts to the mucosal fluid. In embodiments, the composition is frozen or dried (e.g., spray dried/lyophilized) prior to administration to the recipient subject. In embodiments, the composition is not administered to the recipient subject as freshly harvested mucosal fluid, but is frozen or dried (e.g., lyophilized) prior to administration to the recipient subject. In some embodiments, the composition is stored in quarantine until the donor samples comprising the vaginal composition and/or the donor subject have been assessed for the presence of pathogens, and/or transmittable diseases.

The donor is generally healthy, does not exhibit dysbiosis of the vaginal microbiota and optionally is subjected to one or more additional health tests. The vaginal composition provided herein (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises less than 5% of Gardnerella spp., Atopobium spp., and Prevotella spp. For example, the methods of producing a vaginal composition includes a step of providing a microbiota sample (such as a cervicovaginal secretion) from a healthy donor and a step of releasing from quarantine (e.g., based on meeting one or more predetermined (quality) parameters, such as, e.g., those obtained from performing one or more of steps 5(a) to 5(e) below) a processed sample as a (pharmaceutical) composition, e.g., for administration to a recipient in need of a vaginal composition.

Generally, the step of providing and processing of a microbiota sample (such as vaginal mucosal sample or a cervicovaginal secretion) from a healthy donor can include one, two, three, four, five or more steps, and any combinations, e.g., any two, three, four, five or more steps, including: (1) adding a diluent (e.g., saline), buffer, or other excipient to the microbiota sample to create a diluted sample; (2) removing a portion of the diluted microbiota sample for testing (e.g., nucleic acid sequencing); (3) pre-cooling for either refrigeration or freezing of the remainder of the microbiota sample; (4) storing the refrigerated or frozen microbiota sample under quarantine, and/or (5) holding the refrigerated or frozen microbiota sample under quarantine until completion of any combination of (a), (b), (c), (d), and/or (e): (a) testing the donor to exclude the presence of transmissible pathogens (e.g., blood, vaginal swab, and/or urine sample testing), (b) confirming the composition and viability of the donor sample microbiota (e.g., lactobacilli), (c) further confirming the health of the donor by a plurality of post-screening tests occurring within a time period of 30-90 days post-donation (or alternatively: 10-120 days, or 30-60 days), (d) testing for presence of sperm cells, and/or (e) testing pH of the sample (e.g., Figure. 14). Any of steps (1) to (5) and 5(a) to 5(e) are optional and can be carried out sequentially or in parallel in any particular order that is desired.

In one embodiment, the method of producing a vaginal composition comprises A. providing a microbiota sample from a donor's genitourinary tract; wherein step A comprises one, two, or three of steps (1), (2), (3) or any combination thereof, and both steps (4) and (5): (1) adding a diluent to the microbiota sample to create a diluted sample,
(2) removing a portion of the diluted microbiota sample for testing (e.g., nucleic acid sequencing),
(3) pre-cooling for either refrigeration or freezing of the remainder of the microbiota sample, (4) storing the refrigerated or frozen microbiota sample under quarantine,
(5) holding the refrigerated or frozen microbiota sample under quarantine until any completion of any combination of (a) testing the donor to exclude the substantial presence of one or more transmissible pathogens (e.g., blood, and/or cervicovaginal secretions, and/or urine sample testing), (b) confirming the composition and viability of the microbiota, or (c) further confirming the health of the donor by a plurality of post-screening tests occurring within a time period of 30-90 days post-donation; and
B. releasing the refrigerated or frozen microbiota sample from quarantine to define the vaginal composition.

Step (1) may include, e.g., adding an acidifying agent (e.g., to adjust the pH of the sample); adjusting the viscosity of the sample (e.g., to aid administration as described herein); adjusting the isotonicity/osmolarity; and/or adding one or more other active agents, such as described herein, including spermicides, antimicrobial agents, hormonal agents, anti-inflammatory agents, and optionally prebiotics. In one embodiment, the acidifying agent is lactic acid.

For step (2), the microbiota sample (e.g., a sample of vaginal fluid/vaginal secretion) is preferably at least 75 mg or 100 mg, more preferably at least 150 mg, and a portion is removed for nucleic acid sequencing. The microbiota sample may be 75 mg, 100 mg, 150 mg, 200 mg, 250 mg, or 300 mg. In a further embodiment, the microbiota sample (e.g., a sample of vaginal fluid/vaginal secretion) is at least 200 mg, 300 mg, 400 mg, 500 mg, 500 mg, 700 mg or more. In one embodiment, the microbiota sample (e.g., a sample of vaginal fluid/vaginal secretion) is at least 500 mg.

Sequencing is performed to assess the microbial community of the donor microbiota sample and to select suitable donor s. Preferably, the presence of one, two, three, four or five different bacterial species from the genus Lactobacillus is detected in the donor microbiota sample by nucleic acid sequencing. Most preferably, one (dominant) bacterial species from the genus Lactobacillus is detected in the donor microbiota sample by nucleic acid sequencing. In some embodiments, nucleic acid sequencing determines that the donor microbiota sample comprises 80%, about 85%, about 90%, about 91%, about 92%, about 93%, about 94%, about 95%, about 96%, about 97%, about 98%, about 99%, about 99.5%, about 99.9%, 80-99.9%, 75%-95%, 85%-95%, 85%-99.9%, or 90%-99.9% lactobacilli of one species of the total of all detectable species in the preparation. In some embodiments, nucleic acid sequencing determines that the donor microbiota sample comprises 80%, about 85%, about 90%, about 91%, about 92%, about 93%, about 94%, about 95%, about 96%, about 97%, about 98%, about 99.9%, about 99.5%, about 99.9%, 80-99%, 75%-95%, 85%-95%, 85%-99.9%, or 90%-99.9% lactobacilli of more than one species (e.g., two, three, four, or five species) of the total of all detectable species in the preparation. Preferably, nucleic acid sequencing determines that the donor microbiota sample comprises species of Lactobacillus selected from: a) Lactobacillus crispatus; (b) Lactobacillus iners; (c) Lactobacillus jensenii; (d) Lactobacillus gasseri, or any combination thereof (including combinations of two, three, or all four species).

Optionally, nucleic acid sequencing is performed to identify any pathogens or pathobionts in the donor sample, e.g., to determine that a donor sample is substantially free of pathogens and pathobionts. For example, nucleic acid sequencing is preformed to detect the presence of one or more of Gardnerella spp., Atopobium spp., and/or Prevotella spp. In some embodiments, nucleic acid sequencing is preformed to detect the presence of one or more of Gardnerella spp., Atopobium spp., Prevotella spp, and/or Fannyhessa vaginae. In some embodiments, nucleic acid sequencing is performed to detect the presence of one or more of Gardnerella vaginalis, Bacteroides, Mobiluncus spp., Sneathia spp., and Mycoplasma hominis. In some embodiments, nucleic acid sequencing is preformed to detect the presence of one or more of Escherichia, Enterococcus, Pseudomonas, Proteus, Klebsiella, Streptococcus, Staphylococcus, Gardnerella, Ureaplasma, Bacteroides, Peptococcus, Neisseria, Serratia, Corynebacterium, Clostridium, and Candida. The presence of any one or more of these species suggests the displacement of healthy vaginal lactic acid producing bacteria by unwanted species, signaling the presence of vaginal dysbiosis. Donor samples containing more than about 1%, 3%, 5%, 8%, 10% or more than about 15% of species belonging to one or more unwanted species are not used for further processing to generate the vaginal compositions described herein. In one embodiment, donor samples containing more than about 5% of species belonging to one or more unwanted species (e.g., are Gardnerella spp., Atopobium spp., and/or Prevotella spp.) are not used for further processing to generate the vaginal compositions described herein.

Optionally, nucleic acid sequencing is performed to identify the presence of any antimicrobial resistance (AMR) genes in the donor sample, e.g., to determine that a donor sample is substantially free of antimicrobial resistance (AMR) genes. Antimicrobial resistance (AMR) genes include genes that confer resistance to one or more antibiotics, including, e.g., aminoglycosides, beta-lactams, tetracyclines, and sulfonamides (e.g., as described and cataloged in the NCBI National Database of Antibiotic Resistant Organisms (NDARO)). It will be appreciated that due to extensive use of antibiotics the cut-off is not zero. Some reasonable allowance for the presence of AMR genes is made. The precise cut-off can be determined by one of ordinary skill, based on, e.g., the nature of the AMR genes (e.g., degree of health concern) and public health recommendations.

Alternatively, or in addition, tests are performed to determine that a donor sample is substantially free of one or more gram-negative toxins (e.g., endotoxin or lipopolysaccharide (LPS) and other secreted exotoxins and enterotoxins), pathogenicity factors/bacterial virulence factors, and/or colonization factors (e.g., motility, adherence, invasiveness, etc.).

Optionally, tests are performed (e.g., by microscopy) to identify the presence of any human sperm (spermatozoa) in the donor sample, e.g., to determine that a donor sample is substantially free of human sperm (spermatozoa).

Step (3) may include pre-cooling for either subsequent refrigeration (e.g., at 4° C.) or freezing (e.g., −18° C. or −80° C.), depending on the desired time period for storage, quarantine and use for administration. This step may also include freeze-drying (lyophilizing), e.g., for easy storage, packaging, formulation and transport. Further, this step may optionally include viability testing, e.g., upon refrigeration, freezing, or freeze-drying, e.g., viability of the lactobacilli comprised in the vaginal compositions.

Steps (4) and (5) include storing the refrigerated, frozen or freeze-dried microbiota sample under quarantine, and holding the stored microbiota sample under quarantine until completion of a number of tests conducted on the donor microbiota sample and/or the sample donor. The quarantine is lifted, and the sample released for use as a vaginal composition or composition (e.g., pharmaceutical composition), e.g., for administration to a recipient, upon the sample and/or the donor passing one or more predetermined tests (sample quality and/or donor health tests).

Those include one or more of: (a) testing the donor to exclude the presence of transmissible pathogens (e.g., blood, vaginal swab, and/or urine sample testing); (b) confirming the composition and viability of the donor sample microbiota (e.g., lactobacilli), and/or (c) further confirming the health of the donor by a plurality of post-screening tests occurring within a time period of 30-90 days post-donation (or alternatively: 10-120 days, or 30-60 days).

Step (5) testing to exclude the presence of transmissible (and potentially infectious) pathogens (e.g., blood, vaginal swab, and/or urine sample testing) may include determining that the donor is substantially free of any one or more (two or more, three or more, or four or more) of: (i) bacteria involved in bacterial vaginosis (e.g., Gardnerella and Mobiluncus), (ii) yeast (e.g., Candida, Cryptococcus, and Saccharomyces species), (iii) sexually transmitted pathogens (including Neisseria gonorrhea, Chlamydia trachomatis, and Trichomonas vaginalis), (iv) bacteria involved in urinary tract infections (e.g., E. coli, Staphylococcus, Chlamydia, and Mycoplasma), and (v) viruses (e.g., HIV, human papilloma virus (HPV), hepatitis B virus, hepatitis C virus, HSV-2).

Step (5) testing may include determining that the donor is substantially free of any one or more sexually transmitted infections or diseases (STI, STD), including chlamydia, chancroid, crabs (pubic lice), genital herpes, genital warts, Hepatitis B, human immunodeficiency virus/acquired immunodeficiency syndrome, human papilloma virus, trichomoniasis, molluscum contagiosum, pelvic inflammatory disease, syphilis, gonorrhea, and yeast infections.

Step 5 testing may include determining that the donor does not exhibit a dysbiosis in the vaginal tract, e.g., by one or more established tests for bacterial vaginosis (BV) and bacterial infections.

Such tests include Amsel Criteria, Nugent Gram-stain scoring system, and Hay-Ison Criteria.

Alternatively, other methods may be used to determine the absence of dysbiosis, e.g., using the BV Blue test or Affirm Microbial Identification Test.

Amsel Criteria include the presence of three of the following four symptoms: (a) thin homogeneous malodorous discharge; (b) vaginal pH fluid>4.5; (c) an amine odor from vaginal fluid when 10% KOH is added; and (d) the presence of “clue” cells (vaginal epithelial cells with adherent bacteria that obscure cell margins) (Amsel et al., Am. J. Med. 74:14-22 (1983)).

The Nugent Gram-stain scoring system involves assessment of a normally prepared Gram stain for relative abundance of three morphotypes of bacteria, and then calculating the so-called Nugent score based on the amounts of large Gram-positive rods (lactobacilli morphotype; decrease in lactobacilli is scored as 0 to 4), small Gram-negative and variable rods (Bacteroides and Gardnerella morphotype; scored as 0 to 4), and curved gram-variable rods (Mobiluncus spp. morphotype; scored as 0 to 2). The Nugent score can range from 0 to 10, with scores of 0-3 deemed normal (non-BV), 4-6 intermediate, and 7-10 positive for BV.

Hay-Ison Criteria (alternatively Ison-Hay scoring system) suggests five grades of flora: a) Grade 0, epithelial cells with no bacteria; b) Grade I, normal vaginal flora (lactobacillus morphotypes alone); c) Grade II, reduced numbers of lactobacillus morphotypes with a mixed bacterial flora; d) Grade III, mixed bacterial flora only, few or absent lactobacillus morphotypes; e) Grade IV, Gram positive cocci only.

Grades 0, I, and IV are found in women without BV. Grade II is intermediate and not found in women with BV as defined by Amsel criteria. Grade III is consistent with BV as diagnosed by Amsel criteria. Grade III flora are indicative of BV (C. A. Ison and P. E. Hay, Sex Transm. Infect. 2002 December; 78(6):413-5).

The BV BLUE test (Gryphus diagnostics) detects sialidase activity, an enzyme produced by BV-associated bacteria such as Gardnerella vaginalis, Bacteroides spp., Prevotella spp., and Mobiluncus spp. A vaginal fluid sample is placed in the test vessel which contains a chromogenic substrate for sialidase. After incubation, a developer solution is added, and if the sample contained a high level of sialidase, a blue or green color is seen. Samples containing no sialidase, or low levels of this enzyme, will generate a yellow color in the reaction.

The AFFIRM Microbial Identification Test (Beckton Dickinson) is a DNA probe-based diagnostic test for the differential detection and identification of the three types of vaginitis-associated organisms: Candida spp., G. vaginalis and T. vaginalis.

In some embodiments, donors are selected from generally healthy, pre-menopausal women, of ages 18 years and older with regular, predictable menstrual cycles. In some embodiments, donors are selected from generally healthy, post-menopausal women. In some embodiments, donors are selected from both pre- and post-menopausal women. Donors can take oral contraceptives, hormonal contraceptives, hormonal intrauterine devices or no contraceptives. Donors are substantially free of vaginal symptoms, such as odor, discharge, or itching. Optionally, donors do not use or perform one or more of (or all of a-e) during the sample donation period, e.g., from initial donor screening to the final donation: a) use vaginal feminine products that are inserted, e.g. tampons, menstrual cups, sex toys, though sanitary napkins are acceptable; b) use other vaginal products, such as, e.g., cleansing products, spermicides, lubricants, hygiene powders and sprays); c) have vaginal and anal intercourse; d) take baths, go swimming, sit in a hot tub, and/or e) wear thong underwear.

Donors may be excluded if they exhibit one or more of the following (e.g., test above a set of predetermined thresholds, e.g., concerning viral, fungal, and bacterial pathogen and/or pathobiont load, for which individual maximal thresholds may be set, e.g., above zero, such as, e.g., being substantially free thereof): (a) a health history of one or more of: bacterial vaginosis, recurrent yeast infection, trichomoniasis, syphilis, human papilloma virus (HPV) including genital warts, high grade pap smear, herpes, pelvic inflammatory disease, recurrent urinary tract infection, and mycoplasma, or any combination thereof; (b) testing positive for one or more of: HIV, Hepatitis A/B/C, syphilis, Human T-lymphotrophic Virus (HTLV)-I/II, WNV, Epstein-Barr Virus (EBV), rubella, Toxoplasma gondii, Herpes Simplex Virus (HSV)-1/2, chlamydia, gonorrhea, Mycoplasma genitalium, Trichomonas vaginalis, HPV, and other yeast or bacteria that are considered pathogenic/abnormal and/or show antibiotic resistance, or any combination thereof, (c) vaginal fluid/cervicovaginal secretions not dominated by one of the common vaginal lactobacillus species, e.g., as determined by qPCR; and/or (d) history of gonorrhea or chlamydia (e.g., within 12 months prior to screening), or any combination of (a), (b), (c) and (d).

Further, donors may be excluded if they exhibit one or more of the following: hysterectomy, intra-uterine device insertion or removal, cervical cryotherapy, or cervical laser treatment (e.g., within 2 months prior to screening), any condition requiring regular periodic use of systemic antibiotics, use of long-acting hormonal treatments, social, medical, or psychiatric condition, including history of drug or alcohol abuse, menopause (e.g., defined as more than 12 consecutive months of amenorrhea without another known cause), irregular menstrual cycles, use of other medication, or any combination thereof. Preferably, donors are not currently pregnant or breastfeeding.

If desired, donors exhibiting one or more of cytomegalovirus (CMV), Rubella, and Varicella Zoster Virus (VZV) IgG, or any combination thereof, will only be matched with CMV positive and/or Rubella and/or VZV positive recipients, or can be excluded.

### Methods of Administration

Aspects of the invention include methods for vaginal administration to a human subject (e.g., an infertile subject undergoing or planning to undergo an assisted reproductive procedure) of a composition comprising a vaginal composition described herein (e.g., to treat RPL). The methods may include using a device for administration, e.g., these methods would normally be carried out by a healthcare provider (e.g., in a clinic). For example, the composition comprising a vaginal composition is provided frozen, e.g., in a cryo-vial, then thawed and pre-heated to 37° C. and then dispensed into a syringe/applicator by a healthcare provider and then administered to a recipient. Alternatively, the methods include using an alternative dosage form described herein, such as, e.g., a suppository, tablet, capsule, film, cream, etc. The methods can, if desired, be carried out by the recipient herself, e.g., by self-administration (e.g., at home). The methods may also include other healthcare related activities, such as diagnosing a health issue and providing standard of care in addition to providing a vaginal composition or composition described herein.

The activities can include the one or more combination therapies provided herein. For example, the methods for administration described herein may further comprise administering antimicrobial agents, antifungal agents, antibacterial agents, antiviral agents, antibiotics, antiparasitic agents (e.g., with activities against Trichomonas vaginalis), anti-inflammatory agents, and the like.

In embodiments, in which administration is carried out using a device, the device typically includes an open end (e.g., a tip) for insertion into the vaginal cavity, and a dispensing end (e.g., a plunger or piston) to expel the composition through the open end. The administration steps include: a) introducing the open end into a vaginal cavity, b) expelling the composition into the vaginal cavity, c) removing the device from the vaginal cavity (after administering the desired dose). Administration is preferably carried out with the recipient being in a lithotomy position, e.g., with the recipient in a lithotomy position. In some embodiments, the recipient is to remain in a lithotomy position for at least 5 minutes, at least 10 minutes, at least 20 minutes, at least 30 minutes before returning to an upright position to allow the vaginal composition or composition sufficient residence time in the vaginal cavity, e.g., sufficient contact time with the mucosal or endometrial surfaces of the vagina. In some embodiments, the administration is carried out targeting areas high in the vaginal cavity, e.g., near the vaginal fornices.

If desired, the menstrual cycle of the recipient is taken into account when determining the timing of administration. For example, the procedure may be avoided during menstrual discharge. In some embodiments, a time other than during menstrual discharge is preferred for carrying out the administration, including, e.g., during a time window that includes prior to ovulation and prior to menstrual discharge.

In some embodiments, the precise steps, timing, and length of the procedure varies between recipients (and is, e.g., determined by a healthcare provider) in order to provide optimal conditions for the bacteria (e.g., lactobacilli) comprised in the vaginal composition to colonize and become established (engrafted) in the vagina of the recipient.

### Methods of Treatment and Recipient Considerations

Suitable recipients include subjects exhibiting a dysbiosis of the vaginal microbiota and those that are in need of treatment for dysbiosis, such as dysbiosis associated with an infection (e.g., with a pathogen), and/or (chronic) inflammation. In some embodiments, suitable recipients include subjects that are undergoing or planning to undergo an assisted reproductive procedure (e.g., IVF).

Aspects of the invention relate to methods for restoring a healthy human vaginal microbiota balance in the genitourinary tract. The methods include administering to a subject in need of restoration of vaginal microbiota balance an effective amount of a composition comprising a vaginal composition described herein for the purpose of providing a community of microbial species that are capable of colonizing or inhabiting the human vagina or vaginal epithelium and that provide a microbial niche that discourages the growth of pathogenic microbes and is not pro-inflammatory (e.g., is anti-inflammatory). The methods include administering to a subject in need of vaginal microbiota balance an effective amount of a composition comprising a vaginal composition described herein for the purpose of maintaining healthy vaginal microbiota or a healthy vaginal microbiota balance.

In one aspect, the invention provides a composition for use in treating RPL, dysbiosis, and/or inflammation in the genitourinary tract of a human subject, wherein the subject exhibits a dysbiotic microbiota in the genitourinary tract, said method comprising administering to the subject an effective amount of the composition, wherein the composition comprises a vaginal composition, wherein the preparation (i) comprises one, two, three or four bacterial species from the genus Lactobacillus, selected from Lactobacillus crispatus, Lactobacillus iners, Lactobacillus jensenii, Lactobacillus gasseri, which comprise >80-99.9% of all detectable bacterial species of the preparation; and (ii) comprises <5% of Gardnerella spp., Atopobium spp., and Prevotella spp.; optionally wherein the composition comprises a pharmaceutically acceptable carrier or diluent.

Inflammation and disorders, conditions or diseases associated with inflammation are used interchangeably and comprise acute and chronic inflammation. Dysbiotic vaginal microbiota may be associated with inflammation and/or RPL.

The subject may be an asymptomatic subject, wherein an asymptomatic subject is characterized by having no Amsel's criteria, Nugent score or other measures of symptoms associated with bacterial vaginitis (BV). In one aspect, the recipient is a asymptomatic dysbiotic woman.

In some embodiments, a subject is being concurrently administered (or has been previously administered) one or more fertility drugs. In some embodiments, a subject suffering from RPL is being co-treated with one or more fertility drugs. In some embodiments, a fertility drug is intended to promote an artificial insemination (e.g., IUI) or an assisted reproductive procedure (e.g., IVF). A fertility drug may be selected from the group consisting of Clomiphene citrate (Clomid), Human chorionic gonadotropin (hCG), Follicle-stimulating hormone (FSH), Human menopausal gonadotropin (hMG), Gonadotropin-releasing hormone (GnRH), Gonadotropin-releasing hormone agonist (GnRH agonist), Gonadotropin-releasing hormone antagonist (GnRH antagonist), Antagon (ganirelix acetate), Dostinex(cabergoline) and Parlodel.

### Vaginal Wash

In order to reduce the quantity of the pathogens in the vaginal cavity of the dysbiotic recipient, in some embodiments, prior to administering the pharmaceutical composition the vaginal cavity is rinsed, e.g., a vaginal wash is performed.

The vaginal wash may be performed with an absorbent material pre-soaked in the wash solution.

In one embodiment, the absorbent material is gauze. In some embodiments, a tampon-shaped gauze may be used. The wash is typically performed by a medical professional, such as a gynecologist, wherein the recipient is in the lithotomy position and the pre-soaked absorbent material is used with pliers to wash the surface of the vaginal epithelium. After performing the vaginal wash, excess wash solution may be absorbed by a dry absorbent material, e.g., a dry tampon-shaped gauze.

The vaginal wash can be performed with any solution that is suitable for reducing the quantity of pathogens in the vaginal cavity, including but not limited to saline, antiseptic, or lactic acid. In some embodiments, the vaginal wash comprises a saline vaginal wash, lactic acid wash, or wash with an antiseptic solution. Suitable antiseptic solutions include chlorohexidine and povidone-iodine. In a particular embodiment, the vaginal wash is performed with saline. In another particular embodiment, the vaginal wash is performed with lactic acid, optionally wherein the lactic acid has a pH of about 3.5-4.5. In a particular embodiment, the vaginal wash is performed with lactic acid having a pH of about 3.8-4.3 or 3.8 to about 4.0. In a further particular embodiment, the vaginal wash is performed with an antiseptic solution, optionally wherein the antiseptic solution is chlorohexidine or povidone-iodine. In a particular embodiment, the vaginal wash is performed with povidone-iodine, wherein the solution comprises about 10% povidone-iodine. In a particular embodiment, the vaginal wash is performed with chlorhexidine, wherein the solution comprises about 0.5% chlorhexidine.

In some embodiments, a method of the disclosure will be as follows: (i) antiseptic wash; (ii) saline or PBS wash (e.g., to remove antiseptic); (iii) administration of one or more doses of the vaginal composition on 1-3 consecutive days (e.g., up to three total doses per day, i.e., 1, 2 or 3 transplants on 1-3 consecutive days, optionally wherein each dose is preceded by an antiseptic wash); (iv) a waiting period of 1-12 weeks; and (v) implantation of an embryo implant (e.g., a fresh embryo that has never been frozen; or a thawed embryo that has been frozen). In embodiments that involve implantation of a fresh embryo, the embryo transfer is performed usually either three or five days after the retrieval of the egg. In embodiments that involve implantation of a frozen embryo, the embryo has been previously created, sometimes even years earlier, and then will be placed into the uterus after being thawed.

### Homeostasis of Vaginal Microbiota

A healthy human vaginal microbiota balance includes establishment (including, e.g., engraftment) of a select variety of microbial species (including one or more Lactobacillus species) in which the relative numbers of each species or the sum of vaginal Lactobacilli (e.g. Lactobacillus, selected from Lactobacillus crispatus, Lactobacillus iners, Lactobacillus jensenii, Lactobacillus gasseri) are in homeostasis, as measured post administration of a composition comprising the vaginal compositions described herein. Homeostasis, generally, is a state in which the relative abundance of each species in a population does not substantially vary over a given period of time, e.g., at least for 1 day, 3 days, 5 days, 7 days, 10 days, 14 days, 21 days, 30 days, 60 days, 90 days, 6 months, or at least 12 months, or, e.g., over several (1, 2, 3, 4, 5, 6) menstrual cycles in a reproductive age woman. Homeostasis may be measured, e.g., by metagenomic sequencing, such as MLST and MLVA, 16S sequencing and/or qPCR. If desired, CFU counts may be taken, e.g., to measure persistence of colony forming units of healthy bacteria).

A healthy human vaginal microbiota balance at a homeostatic state (preferably rich in lactobacilli, e.g., at least 50%, 60%, 70% or at least 80% of lactobacilli, e.g., selected from one or more of: L. crispatus, L. iners, L. gasseri and L. jensenii) confers, e.g., resistance to perturbations caused by vaginal pathogens (non-pathogenic) and/or provide an anti-inflammatory environment and is stable for a period of time. However, a healthy vaginal microbiota does not require complete absence of all pathogens. Many healthy women have low levels of yeast and/or pathobionts present in their microbiota.

In some embodiments, the methods for restoring a healthy human vaginal microbiota balance in the genitourinary tract include providing a non-proinflammatory or anti-inflammatory environment in the genitourinary tract. In some embodiments, the compositions comprising a vaginal composition described herein when administered (e.g., for treatment) provide anti-inflammatory activity to the genitourinary tract. In some embodiments, the compositions comprising a vaginal composition described herein when administered (e.g., for treatment) do not provoke a local or systemic inflammatory or immune response in the subject or recipient. In some embodiments, the compositions comprising a vaginal composition described herein when administered (e.g., for treatment) promote a local or systemic anti-inflammatory or immune response in the subject or recipient. In some embodiments, the compositions comprising a vaginal composition described herein when administered (e.g., for treatment) provide maintenance of the balance of anti-inflammatory and proinflammatory mediators in vaginal epithelial cells of the genitourinary tract.

### Inflammatory Markers

Proinflammatory cytokines that can be modulated by the methods described herein include, e.g., IL-1β, IFN-γ, IL-2, IL-4, IL-6, IL-8, IL-10, IL-12p70, IL-13, IL-17, IL-23, and TNF-α. In one embodiment, the administration of the vaginal composition effects reducing or stabilizing the levels of inflammatory markers, wherein the markers comprise one or more of IL-1a, IFN-γ, IL-18, IL-12, and MMP-10. In some embodiments, anti-inflammatory markers are increased after administration of the preparation of the invention.

Anti-inflammatory and proinflammatory mediators upon administration and engraftment of the lactobacilli comprised in the vaginal compositions described herein can be measured (e.g., collecting a vaginal sample (e.g., vaginal fluid/secretion) or a systemic sample, (e.g., blood) using suitable commercially available biomarker panels.

Provided herein are methods of treating (chronic) inflammation of the genitourinary tract. The methods include administering to a subject in need of treatment an effective amount of a composition comprising a vaginal composition described herein. Subjects treated by the methods described here includes exhibiting an inflammation (in some instances chronic inflammation) that may result in a number of adverse health outcomes, such as urinary tract infections (UTIs).

### Dysbiosis

Provided herein are methods of treating dysbiosis of the genitourinary tract. The methods include administering to a subject in need of treatment an effective amount of a composition comprising a vaginal composition described herein. Subjects treated by the methods described here includes exhibiting an imbalance in the vaginal microbiota that may result in overgrowth of pathogenic microorganisms and pathobionts, resulting in dysbiosis, inflammation, and/or infections. The methods include the treatment of vaginal infections (vaginitis) such as bacterial vaginosis, vaginal candidiasis and trichomoniasis and combinations thereof with composition comprising a vaginal composition described herein.

Vaginal dysbiosis is a prevalent condition that occurs in about 30-40% of adult female subjects.

In some embodiments, the dysbiotic subject does not exhibit any symptoms (asymptomatic). In other embodiments, the dysbiotic subject suffers from vaginal symptoms characterized by Amsel's criteria, Nugent score criteria or Hay-Ison criteria.

Bacterial vaginosis (BV) is the most common vaginal infection and is associated with complications during pregnancy as well as an increased risk for sexually transmitted diseases. It is caused by an imbalance of naturally occurring bacterial flora, wherein the lactobacilli are overgrown by a mixed flora of anaerobic bacteria. Amongst other diagnostic criteria, a pH greater than 4.5 is thought to be suggestive of bacterial vaginosis. BV may be diagnosed using Amsel criteria, Nugent score, Hay-Ison criteria, or another suitable method of diagnosis. Current treatment options rely on oral or vaginal administration of classical antibiotics such as metronidazole and clindamycin, however there is a high rate of recurrence of over 50% within 3 months after the first exposure. Alternative treatment options include acidification of the vagina with naturally occurring acids such as lactic acid or acetate.

In one embodiment, a subject exhibiting one or more diagnostic signs of BV is treated with an effective amount of a composition comprising a vaginal composition described herein. In one embodiment, treatment comprising administration of an effective amount of a composition comprising a vaginal composition further comprises a combination therapy (as described herein), including providing standard of care treatment for BV, such as, e.g., administering antibiotics such as metronidazole and clindamycin and/or acidifying the vagina with an acidifying agent, such as, e.g., lactic acid or acetate, either prior to, concomitant with or after administration of the composition comprising a vaginal composition to the subject.

Vaginal candidiasis is a fungal infection of any of the Candida species (yeasts), of which Candida albicans is the most common. Most Candida infections are treatable and result in only minimal complications such as redness or itching. However, complications may also be severe or even fatal if left untreated in certain populations, such as immuno-compromised patients.

External use of detergents or internal disturbances (hormonal or physiological) can disturb the normal vaginal flora and result in an overgrowth of Candida cells causing infection. Pregnancy and the use of oral contraceptives have also been reported as risk factors. In clinical settings, candidiasis is commonly treated with antimycotics such as clotrimazole, nystatin, fluconazole and ketoconazole. However, certain yeasts such as, e.g., C. albicans can develop resistance to said antimycotic drugs.

In one embodiment, a subject exhibiting one or more diagnostic signs of candidiasis is treated with an effective amount of a composition comprising a vaginal composition described herein. In one embodiment, treatment comprising administration of an effective amount of a composition comprising a vaginal composition further comprises a combination therapy (as described herein), including providing standard of care treatment for candidiasis, such as, e.g., administering antimycotics such as, e.g., clotrimazole, nystatin, fluconazole and/or ketoconazole, either prior to, concomitant with or after administration of the composition comprising a vaginal composition to the subject.

Trichomoniasis is a sexually transmitted disease of the urogenital tract and is caused by the parasite Trichomonas vaginalis. Symptoms include inflammation of the cervix, urethra and vagina, which produce an itching and burning sensation. Treatment options include the use of antibiotic/anti-protozoal (e.g., metronidazole) or anti-parasitic (e.g., tinidazole) drugs. However, as with most anti-microbial drugs, resistance may occur.

In one embodiment, a subject exhibiting one or more diagnostic signs of Trichomoniasis is treated with an effective amount of a composition comprising a vaginal composition described herein. In one embodiment, treatment comprising administration of an effective amount of a composition comprising a vaginal composition further comprises a combination therapy (as described herein), including providing standard of care treatment for Trichomoniasis, such as, e.g., administering antimycotics such as, e.g., antibiotic/anti-protozoal (e.g., metronidazole) or anti-parasitic (e.g., tinidazole) agents, either prior to, concomitant with or after administration of the composition comprising a vaginal composition to the subject.

In one aspect, the invention provides a pharmaceutical composition comprising the vaginal composition of the invention. The pharmaceutical composition may comprise the vaginal composition and a pharmaceutically acceptable excipient.

### Definitions

The definitions hereinafter apply to all aspects disclosed herein. For example, the term “vaginal composition(s)” shall have the same meaning when used in the context of the first aspect which pertains to the vaginal composition for use in a method of treating RPL, or when used in the context of the second aspect which pertains to a method of producing a vaginal composition, or when used in the context of the third aspect which pertains to a pharmaceutical composition comprising said vaginal composition, or when used in the context of any other aspect disclosed herein.

Unless otherwise explained, all technical and scientific terms used herein have the same meaning as commonly understood by one of ordinary skill in the art to which this disclosure belongs. It is further to be understood that methods and materials similar or equivalent to those described herein can be used in the practice or testing of this disclosure. The materials, methods, and examples are illustrative only and not intended to be limiting. Other features of the disclosure are apparent from the following detailed description and the claims.

The singular terms “a,” “an,” and “the” include plural references unless context clearly indicates otherwise. Similarly, the word “or” is intended to include “and” unless the context clearly indicates otherwise. The term “comprises” means “includes.” The abbreviation, “e.g.” is derived from the Latin exempli gratia, and is used herein to indicate a non-limiting example. Thus, the abbreviation “e.g.” is synonymous with the term “for example.” The term “optional” or “optionally” means that the subsequent described event, circumstance or substituent may or may not occur, and that the description includes instances where the event or circumstance occurs and instances where it does not.

Unless otherwise indicated, all numbers expressing quantities of components, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art.

In a preferred embodiment, the term “about” shall allow a deviation of ±10%, and even more preferably of ±5% from an indicated numerical value.

The term “comprising” shall be understood to simultaneously also disclose the term “consisting” as a preferred option. For example, if a composition is said to comprise three components, this also discloses a composition consisting of these three components as preferred embodiment.

If the term “comprising” is used when referring to (a) pharmaceutically active compound(s), bacterium(a), and the like, this shall be understood to simultaneously also disclose that the pharmaceutically active compound(s), bacterium(a), and the like is/are preferably the sole pharmaceutically active compound(s), bacterium(a), and the like. For example, if a donor sample is said to comprise Lactobacillus crispatus and Lactobacillus iners, this simultaneously and preferably discloses that a donor sample contains Lactobacillus crispatus and Lactobacillus iners as the sole bacteria. If a donor sample is said to comprise Lactobacillus crispatus, Lactobacillus iners, and an excipient, this simultaneously and preferably thus discloses that a donor sample contains Lactobacillus crispatus and Lactobacillus iners as the sole bacteria and in addition comprises an excipient. It further discloses, that the donor sample consists of Lactobacillus crispatus, Lactobacillus iners, and an excipient.

All patents, patent applications, and publications identified are expressly incorporated herein by reference for the purpose of describing and disclosing, for example, the methodologies described in such publications that might be used in connection with the present invention.

“Genitourinary tract” or “urogenital tract” as used herein as used herein includes the uterus, fallopian tubes, ovaries, vagina, cervix, vulva, endometrium, and urinary tract. In some instances, used herein are teachings and exemplifications specifically calling out the “vaginal tract”, “vaginal cavity” “reproductive tract” or “vagina”. In some embodiments, the genitourinary tract comprises the reproductive tract. One of ordinary skill will appreciate that these exemplifications and teachings are illustrative only and non-limiting and, thus also apply, where appropriate, to other anatomical sites of the genitourinary tract or urogenital tract, not just to the vagina.

“Dysbiosis” as used herein means a microbial imbalance where normally predominant species are diminished in abundance and less predominant species become more abundant and/or predominant. Vaginal dysbiosis is a microbial imbalance in the vagina, an aberration of the healthy state. Dysbiosis is generally associated with one or more of: (a) qualitative and quantitative changes in the content or amount of the microbiota itself, (b) changes in their metabolic activities; and/or (c) changes in their local distribution. A dysbiotic human vaginal microbiota balance refers to a population of vaginal microbes that promotes inflammation of a tissue of the vagina and/or that contributes to or establishes an environment that permits or promotes the colonization or growth of one or more pathogenic microbes. Dysbiosis also refers to a perturbation of the vaginal homeostasis. In some embodiments, a dysbiotic vaginal microbiota will generally result in increased pH relative to a healthy microbiota, e.g., a pH above 4.5, e.g., a pH of 5.0, 5.5, 6.0, 6.5, 7.0 or higher. In some embodiments, vaginal dysbiosis is characterized by a reduction of Lactobacillus spp., and an increased diversity of vaginal anaerobic bacteria. Vaginal dysbiosis is associated with upper genital tract infections or pelvic inflammatory disease (PDI), and increased risk of sexual transmitted diseases. Dysbiosis may be characterized by the relative amount of selected pathogens, such as >20% selected pathogens, and the relative low abundance of vaginal lactobacilli, e.g. <10% vaginal lactobacilli (L. crispatus, L. iners, L. jensenii, L. gasseri).

Dysbiotic subjects as used herein comprise subjects having vaginal symptoms (symptomatic subjects) and subjects not having any vaginal symptoms (asymptomatic subjects), wherein symptoms are characterized by Amsel's criteria, Nugent score and/or Hay/Ison score.

In contrast, the term “normal” or “healthy” “vaginal flora” or “vaginal niche” “microbiota” or “state” or similar terms connote that a woman has no vaginal complaints and does not exhibit a vaginal pathology (e.g., no sign or symptom corresponding to or resulting from a pathology of the vagina), and the condition of the vagina is such of a relatively low susceptibility to sexually transmitted diseases and pathogens, and generally of low pH, e.g., less than or equal to pH 4.5, e.g., between 3.2 and 4.5; and generally dominated by lactic acid producing bacteria (e.g., Lactobacillus spp.). Normal vaginal microbiota or normal flora are a community of microorganisms that localize to the vagina in a normal, healthy, that is, a non-pathological, non-pathogenic and/or non-dysbiotic, state.

“Cervicovaginal secretions” (“CVS”) or “vaginal fluid” or “vaginal mucosal sample” refers to the mixture of mucus secreted by the cervix, shed epithelial cells, vaginal transudate, and bacteria found in the vagina of a woman.

“Isolated bacterial strain” means a strain that has been separated from other strains (e.g., from a vaginal bacterial community, e.g., derived from a sample of cervicovaginal secretions or vaginal fluid) and cultivated in vitro in a culture comprising said strain. An isolated bacterial strain is substantially free of contaminants or components that accompany the material it was derived from in its native state (e.g., such as, vaginal mucus and epithelial cells).

“Culture-independent method” means methods not involving isolation and/or in vitro propagation of bacterial strains, e.g., in cultures.

The term “microbe” is used synonymously with the term “microorganism” and includes bacteria (Archaea, Eubacteria), yeast, fungi, and viruses. The term “species” is used herein to refer to a taxonomically and/or genetically distinct group of microorganisms. Species may include one or more distinguishable (e.g., by sequencing) strains.

The term “microbiota” refers to a community of microorganism localized to a distinct shared environment (a “microbial niche”). For example, “vaginal microbiota” is a community of one or more species of microorganisms that are localized to, or found in, a vagina. The term “microflora” or “flora” is used synonymously with the term “microbiota.” Healthy or normal microbiota denominates the community of commensal microorganisms that colonize (inhabit) a particular microbial niche of the host, such as the vagina. Bacteria are the most numerous microbial components of the normal flora.

“Mucosa” as used herein indicates a mucous membrane. Mucus is a secretion produced by, and covering, mucous membranes. Mucous fluid is viscous and typically produced from mucous cells (e.g., goblet cells) found in mucous membranes and submucosal glands, and rich in antiseptic enzymes (such as lysozyme), immunoglobulins, inorganic salts, proteins such as lactoferrin, and glycoproteins (mucins). Mucosal surfaces include epithelial linings of the reproductive tract (vagina) and, e.g., lactobacilli are capable of colonizing the vaginal mucosal surfaces.

As used herein, the term “effective amount” means the amount (e.g., of a composition or preparation) to be administered to a typical subject (e.g., a recipient) that is sufficient to lead to a desired beneficial or therapeutic effect in the subject. The desired beneficial or therapeutic effect includes prophylaxis and/or treatment, e.g., of dysbiosis, inflammation or an infection or urogenital tract, and also includes the restoration and/or rebalancing of the vaginal microbiota (e.g., to achieve homeostasis), e.g., an anti-inflammatory and/or anti-pathogenic state of the urogenital tract and the vaginal microbiota. An effective amount to treat RPL includes the amount necessary to revert vaginal or cervicovaginal dysbiosis and promote a successful pregnancy, e.g., resulting in live birth. An effective amount, for example, is the amount sufficient (e.g., at dosages and for periods of time necessary) to alleviate at least one or more symptom, delay the development of a symptom, alter the course of a symptom (e.g., slowing the progression of a symptom), or reverse a symptom. Effective amounts generally cause statistically significant, measurable changes.

The effective amount in a delivery system varies depending on the particular agent, intended use, expected release rate and the time for which the system is expected to provide therapy. A variety of devices with varying sizes can be formulated for administering dosages. A person skilled in the art is readily able to determine the effective amount of the active agent needed for each specific application and delivery system. Effective amounts can be determined, e.g., in clinical trials and animal studies. The term “effective amount” is used interchangeably with the term “therapeutically effective amount”.

A “lyophilized” or “freeze-dried” composition refers to a composition from which moisture has been removed, e.g., for easy storage and transport. Such compositions can be rehydrated before use (e.g., administration).

As used herein, the term “viable” refers to a cell (e.g., a bacterial cell) that is able to survive in a given condition (e.g., storage for a certain period of time under particular storage conditions, e.g., including, temperature, humidity) and is generally able to colonize and reproduce (e.g., in the urogenital tract) after exposure to the condition. Percent viability refers to the percentage of viable cells in a population. For example, percent viability can refer to the percentage of lactobacilli in a pharmaceutical composition that will survive (e.g., refrigeration, freezing and/or storage) and colonize upon application to a vaginal mucosal surface. Further, “viable” in the context of a “viable neonate” or “viable embryo” refers to a neonate or embryo that can survive and live in utero as well as outside the womb, i.e. after birth. A “viable birth” is also interchangeably used with the term “live birth”.

The abbreviation “cfu” means “colony-forming unit”.

The abbreviation “VCC” means viable cell count (as determined by life cell staining).

The phrases “excipient” “pharmaceutically acceptable carrier” “diluent” or “buffer” as used herein mean a non-active, pharmaceutically acceptable material, ingredient, composition or vehicle that is added to form part of the final formulation and/or maintains a drug or other agent in a form for delivery to a subject. Each carrier preferably is compatible with the other ingredients of the formulation, for example the carrier does not decrease the impact of an active ingredient or agent upon the treatment, e.g., the carrier is pharmaceutically inert. In some embodiments a pharmaceutically acceptable carrier can be a carrier other than water (including, e.g., a cream, emulsion, gel, liposome, nanoparticle, film, ointment and/or vaginal device). In some embodiments, a pharmaceutical composition is provided comprising the vaginal composition together with a pharmaceutically acceptable carrier and/or diluent (e.g., saline).

These compositions allow the easy administration of the preparations by means known to the person skilled in the art. In some embodiments, a buffering agent is added, e.g., a weak acid or base that maintains the acidity at a chosen level (e.g., between pH 3.5 and 4.5) and prevents a rapid change in acidity.

The terms “contacting”, “administering” or “subjecting” and more specifically, “vaginal application” or “vaginal administration”, are used interchangeably herein and relate to a subject (e.g., a recipient) or a specific organ or other physiological site (e.g., the urogenital tract or vaginal cavity or a subpart thereof, e.g., to a site on a vaginal wall (mucosal or endometrial surfaces) or vaginal fornices) and a device, dosage form or pharmaceutical composition that is provide or given to the subject or site, e.g., for the purpose of colonizing and engrafting a desired bacterial community (e.g., comprising lactobacilli), e.g., as comprised in the vaginal compositions described herein. In one embodiment, administering is performed locally. In one embodiment, administering is performed vaginally, e.g., to improve vaginal health, e.g., in the context of treatment of a disease or disorder.

As used herein, the terms “pharmaceutical composition” or “therapeutic composition” refer to the active agent (e.g., the vaginal compositions described herein) in combination with a pharmaceutically acceptable carrier, e.g., a carrier commonly used in the pharmaceutical industry, or an additional active agent. Pharmaceutical compositions are physiologically and pharmacologically acceptable. Generally, compounds, materials (devices), compositions, and/or dosage forms are “pharmaceutical” “therapeutic” or “pharmaceutically acceptable” if they are, within sound medical judgment (e.g., by a physician or regulatory agency), suitable for use in contact with the tissues of human beings without excessive toxicity, irritation, allergic response, or other problem or complication, commensurate with a reasonable benefit/risk ratio (e.g., desired benefit(s) versus side effects (adverse events)). A pharmaceutical composition will generally comprise agents for buffering and preservation in storage, and can include buffers and carriers for appropriate delivery, depending on the route of administration. Pharmaceutical compositions can be conventionally administered in a unit dose. The term “unit dose” refers to physically discrete units suitable as unitary dosage for the subject, each unit containing a predetermined quantity of active material calculated to produce the desired therapeutic effect in association with the required physiologically acceptable diluent, i.e., carrier, or vehicle.

The terms “dose” and “dosage” are used interchangeably herein. A dose refers to the amount of active ingredient given to an individual at each administration. The dose will vary depending on a number of factors, including frequency of administration (e.g., daily, one or more times per week, per month, or per 3 months); size and tolerance of the individual; severity of the condition; intended result (e.g., treatment, prophylaxis, modulation or restoration of the microbial community), and the route of administration. A baseline dose can be administered and modified based on the initial response of the subject. For example, a single dose of the compositions comprising the vaginal compositions described herein can be in the range of 10⁴ to 10¹⁰ colony forming units (per dose). In other embodiments, a single dose of the composition can be in the range of 10³ to 10¹², 10⁴ to 10⁹, 10⁵ to 10⁹, 10⁵ to 10⁸, 10⁶ to 10⁹, 10⁷ to 10⁹, or 10⁷ to 10¹⁰ colony forming units (per dose).

A “dosage form” refers to a particular physical form of a pharmaceutical composition and depends, e.g., on the dose required to deliver a desired amount of an active agent and on the route of administration. For example, a dosage form can be in a suppository, a tablet, a capsule, a film, a cream, etc., or a device, such as, e.g., an applicator or dispenser, e.g., for vaginal administration. Dosage forms may be single or multiple-use dosage forms.

As used herein, the terms “treat,” “treatment,” “treating” refer to prophylactic and therapeutic treatments, wherein the object is to prevent, reverse, alleviate, ameliorate, inhibit, slow down or stop the progression or severity of a condition or symptom of a condition associated with a disease or disorder. Treatment of recurrent pregnancy loss (RPL) may refer to treatment of one or more symptoms or conditions associated with RPL. In some embodiments, treatment of RPL refers to treatment of an inflammation-associated cause of RPL. In some embodiments, treatment of RPL refers to treatment of dysbiosis in the vaginal microbiome or inflammation in the vagina and/or endometrium. Treatment includes the improvement of symptoms or markers (of the disease or condition) and the cessation or at least slowing of progress or worsening of symptoms that would be expected in absence of treatment. Treatment effectiveness can be measured by monitoring one or more symptoms or clinical markers and is compared, e.g., to the subject's condition and symptoms before administration or to a control subject not undergoing treatment.

In one example, treating a vaginal infection refers to reducing the amount of the infective agent (e.g., number of cells or viral particles), reducing the severity of symptoms, and/or reducing the frequency of symptoms. A treatment that reduces the level of a pathogen to one which is kept in check by the immune system or by the state established by a healthy vaginal microbiota (e.g., the infection is no longer detectable, e.g., by symptoms or general diagnostic techniques) is considered effective as the term is used herein. Treatment of RPL can comprise pregnancy duration until live birth, a prevention or reduction of the frequency of pregnancy loss (e.g., stillbirth), or (increased rate of) occurrence of live birth.

As used herein, the term “colonization” or “engraftment” refers to the colonization of an environment (e.g., a microbial niche), e.g., the vagina or vaginal epithelium, by a microbe, e.g., a bacterium (e.g., lactobacilli), such that the viable population of that microbe continues to reside, e.g., in the niche, for a certain period of time. Engraftment can be transient or stable depending on the period of time the microbe continues to reside in the niche. Colonization and engraftment (and residence time) can be quantified, e.g., by counting the number of colony forming units (CFU)/gram and/or performing nucleic acid sequencing of microbes comprised in one or more vaginal samples that are taken over a certain period of time.

The term “subject” refers to a human (e.g., a human female) subjected to a treatment, observation or study. In one embodiment, the subject is a recipient of a vaginal composition described herein. In one embodiment, the subject is a donor providing a microbial sample. In some embodiments, the subject has one or more of RPL-associated conditions. In some embodiments, the subject has bacterial vaginosis (BV). In some embodiments, the subject has pelvic inflammatory disease (PID). In some embodiments, the subject has antiphospholipid syndrome (APS). In some embodiments, the subject has thrombophilia. In some embodiments, the subject has coeliac disease. In some embodiments, the subject has uterine abnormalities. In some embodiments, the subject has hyperprolactinaemia. In some embodiments, the subject has increased inflammation. In some embodiments, the subject has thyroid malfunction. In some embodiments, the subject has polycystic ovary syndrome (PCOS). In some embodiments, the subject has aberrant insulin metabolism. In some embodiments, the subject has idiopathic (unexplained) RPL. In some embodiments, the subject having RPL may be undergoing an assisted reproductive procedure (e.g., in vitro fertilization). In some embodiments, the subject is undergoing intrauterine insemination or treatments to stimulate egg production. A subject may be an asymptomatic dysbiotic subject (e.g., having a dysbiotic vaginal microbiome but is not exhibiting symptoms (e.g., clinical symptoms) of bacterial vaginosis or a vaginal infection (e.g., a vaginal yeast infection)). In some embodiments, the subject is an asymptomatic dysbiotic subject, or the population of subjects is a population of asymptomatic dysbiotic subjects. The subject may be 15-34 years of age, 35-50 years of age, 51-75 years of age, 15-44 years of age, 35-44 years of age, or 45 or more years of age. In some embodiments, the subject is a pre-menopausal subject.

The term “vaginal composition” refers to a composition obtained from the vaginal cavity of a female subject, wherein the vaginal composition comprises (i) one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the vaginal composition; (ii) less than 5% of Gardnerella spp., Atopobium spp., and Prevotella spp.; and (iii) vaginal transudate and/or mucus, optionally wherein the mucus is cervicovaginal mucus. The vaginal composition can be isolated or processed. The vaginal composition may comprise substantially all, e.g., at least 80%, 90%, 95%, 99%, 99.9% or 100% of all detectable bacterial species of the vaginal microbiota. A vaginal composition sample can be obtained from a healthy, non-dysbiotic subject, e.g., a (putative) donor subject to obtain a donor vaginal composition suitable for administration to a recipient subject for treating RPL. A vaginal composition sample may also be obtained from a subject having RPL and exhibiting dysbiosis in the genitourinary tract, e.g., vaginal dysbiosis or cervicovaginal dysbiosis.

Such a vaginal composition is not suitable for administration but can be used for monitoring the degree of dysbiosis prior to treatment, and the reversion of the dysbiosis after administration of the donor's vaginal composition. In one embodiment, the vaginal composition is obtained by a culture-independent method. In one embodiment, the vaginal composition is an isolated vaginal composition. In one embodiment, the vaginal composition is a screened and/or processed vaginal composition, e.g., is free of or substantially free of pathogens. In one embodiment, the vaginal composition has been diluted with saline or lactic acid. If not indicated otherwise, “vaginal composition” refers to the processed, screened vaginal composition of a healthy subject, wherein the vaginal composition comprises (i) one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the vaginal composition; (ii) less than 5% of Gardnerella spp., Atopobium spp., and Prevotella spp.; and (iii) vaginal transudate and/or mucus, optionally wherein the mucus is cervicovaginal mucus.

In one embodiment, the vaginal composition may comprise one dominant Lactobacillus species.

In another embodiment, the vaginal composition may comprise two dominant Lactobacillus species, wherein dominant refers to being present in a relative amount of >50% of all detectable bacterial species in the vaginal composition. The vaginal composition is generally screened for the presence of pathogens and its bacterial composition prior to administration. The screening and processing of the vaginal composition may further comprise biobanking and testing of suitable recipient-donor matches, e.g., by performing a cell diffusion assay. In some embodiments, about 80-99.9% of all detectable bacterial species of the vaginal composition consist of
(a) Lactobacillus crispatus;
(b) Lactobacillus iners;
(c) Lactobacillus crispatus and Lactobacillus iners;
(d) Lactobacillus crispatus and Lactobacillus jensenii;
(e) Lactobacillus crispatus, Lactobacillus iners and Lactobacillus jensenii;
(f) Lactobacillus crispatus, Lactobacillus gasseri and Lactobacillus jensenii;
(g) Lactobacillus iners and Lactobacillus jensenii;
(h) Lactobacillus iners, Lactobacillus jensenii, and Lactobacillus gasseri;
(i) Lactobacillus iners and Lactobacillus gasseri; or
(j) Lactobacillus crispatus, Lactobacillus iners, Lactobacillus jensenii, and Lactobacillus gasseri.

The term “RPL” (recurrent pregnancy loss) refer to a subject who had two or more pregnancy losses. In the context of the present invention, the subject having RPL further exhibits a dysbiotic microbiota in the genitourinary tract. In some embodiments, the subject having RPL had two or more pregnancy losses, e.g., 2, 3, 4, 5, 6 or more pregnancy losses. In some embodiments, RPL is accompanied by or at least in part caused by bacterial vaginosis (BV). In some embodiments, RPL is accompanied by or at least in part caused by pelvic inflammatory disease (PID). In some embodiments, RPL is accompanied by or at least in part caused by antiphospholipid syndrome (APS). In some embodiments, RPL is accompanied by or at least in part caused by thrombophilia. In some embodiments, RPL is accompanied by or at least in part caused by coeliac disease. In some embodiments, RPL is accompanied by or at least in part caused by uterine abnormalities. In some embodiments, RPL is accompanied by or at least in part caused by hyperprolactinemia. In some embodiments, RPL is accompanied by or at least in part caused by increased inflammation. In some embodiments, RPL is accompanied by or at least in part caused by thyroid malfunction. In some embodiments, RPL is accompanied by or at least in part caused by polycystic ovary syndrome (PCOS). In some embodiments, RPL is accompanied by or at least in part caused by aberrant insulin metabolism. In some embodiments, the subject has idiopathic (unexplained) RPL

The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “lower”, “reduced”, “reduction” or “decrease”, “down-regulate” or “inhibit” mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level (or levels below the limit of detection) as compared to a reference sample), or any decrease between 10-100% as compared to a reference level.

The term “substantially” means to a great or significant extent. For example in the case of a sample that is “substantially free of” pathogens, the term means the sample is, for the most part, or essentially, but possibly not completely, void of a pathogen. A sample that is substantially free of selected pathogens can refer to a sample that comprises about 5%, <5%, <4%, <3%, <2%, or <1% of the selected pathogens. In a particular embodiment, the term “substantially free” comprises <5%.

The term “inflammation”, “inflammatory disorder”, “diseases associated with inflammation”, “conditions associated with inflammation” are used interchangeably and describe any condition, disorder, disease or state that comprise or are characterized by inflammation. The inflammation may be an acute inflammation or a chronic inflammation.

The term “embryo implantation” refers to the successful implantation of the blastocyst embryo into the receptive endometrial (uterine) lining.

The term “pregnancy” can refer to biochemical pregnancy or clinical pregnancy. A biochemical pregnancy is defined, in some embodiments, as a positive Human Chorionic Gonadotropin (beta-CG) test (e.g., in the blood of the subject) at a defined period after embryo transfer or conception (e.g., 2 weeks after embryo transfer). A clinical pregnancy is defined, in some embodiments, by the presence of at least one gestational sac (e.g., intrauterine gestational sac) at a defined time point (e.g., 5-6 weeks after embryo transfer or conception). A “successful pregnancy” refers to a pregnancy that results in a live birth of a neonate. Such live birth includes but is not limited to a full-term birth.

The term “in vitro fertilization (IVF) cycle” refers to a first IVF cycle and subsequent IVF cycles. A first IVF cycle involves stimulation of the subject to produce one or more eggs, extraction of the produced eggs, fertilization of the eggs with sperm, and then a transfer of one or more of the fertilized eggs to the uterus of the subject. A subsequent IVF cycle may be a frozen embryo transfer (FET). In some embodiments, a subsequent IVF cycle involves a second, third, fourth, fifth, or more round of: stimulation of the subject to produce one or more eggs, extraction of the produced eggs, fertilization of the eggs with sperm, and then a transfer of one or more of the fertilized eggs to the uterus of the subject.

The term “predetermined” is used herein to denote threshold values of (i) levels of a (one or more) pathogen, as desired (e.g., a tolerated maximum amount, e.g., as considered safe for a recipient subject, e.g., as determined by regulatory bodies such as FDA and EMA); (ii) a level of microbial viability and/or quantity, e.g., of desired vaginal microbes, e.g., a minimum level in a given composition or dosage form, e.g., determined to be required to make up the minimum effective dose for a treatment described herein (e.g., upon processing of the CVS, a minimum of 50%, 60%, 70% or more of cells remain viable in a composition, or a desired total value of CFU/VCC in the vaginal composition as described herein, or a desired total concentration in the composition, e.g., as expressed in CFU/ml or VCC/ml); (iii) a predetermined weight or volume of the composition, e.g., wherein the desired weight or volume is determined by the form, shape, size of the dosage form and/or the desired concentration (e.g., CFU/ml) or total microbial content (e.g., CFU or VCC) for an effective dose. The predetermined values may be selected to standardize the vaginal composition, dosage forms and pharmaceutical compositions comprising the same. In embodiments, the standardization (e.g., by using the predetermined values and thresholds described herein and then releasing the composition for use) is required for, e.g., manufacturing under good manufacturing practice (GMP) and/or regulatory approval, e.g., by a regulatory body, such as FDA and EMA, e.g., as a regulated product, such as a product regulated under pharmaceutical or cell and tissue regulations. Other predetermined values described herein can include, e.g., pH, osmolarity and/or viscosity of the compositions as desired, e.g., to improve efficacy, mode and/or ease of administration, etc. Predetermined level absence of pathogens: Assessing the pathogens that may be comprised in the vaginal composition ensures that the composition is suitable to be administered to exert a beneficial effect on the recipient subject and does not cause an infection in the recipient subject. In embodiments, the method of obtaining the vaginal composition thus comprises a step of assessing the level of pathogens and releasing the composition only if a predetermined level of pathogens is obtained. The predetermined level of pathogens may be the absence of substantially absent level of pathogens. In embodiments, the predetermined level of pathogens allows up to 5%, 4%, 3%, 2%, 1%, or 0.5% pathogens of the microbial composition to be comprised. The predetermined levels of the pathogens are not effective or causative for an infection in a recipient subject. In some embodiments, the pathogens comprise pathobionts, which can be commensal in nature but can also become pathogenic (e.g., can cause or promote disease) when certain genetic and/or environmental conditions are present in a subject. In embodiments, the predetermined level of pathobionts is <30%, <20%, <10%, <5%, <2%, <1%, <0.5%, or <0.1% pathobionts in the vaginal composition. Predetermined viability and or quantity ofvaginal or urogenital microbes: The method of obtaining the vaginal composition may comprise a step of assessing the viability and/or quantity of vaginal or urogenital microbes comprised in the preparation, and releasing the preparation only if a predetermined level of viability and/or quantity of the vaginal or urogenital microbes is obtained.

In embodiments, the predetermined viability and/or quantity may be assessed with live/dead staining or by determining colony forming units or viable cell counts (CFUs or VCCs). In embodiments, the predetermined viability is 10³ to 10¹⁵ colony forming units or viable cell counts (CFUs or VCCs), 10⁴ to 10¹¹ CFUs/mL or VCCs/mL, 10⁵ to 10¹¹ CFUs/mL or VCCs/mL, or 10⁶ to 10¹¹ CFUs/mL or VCCs/mL. In embodiments, the predetermined viability is a threshold value of 50%, 60%, 70% or more viable cells in the vaginal composition. Predetermined level of pH: The vaginal mucosal pH is a clinical parameter that in subjects with vaginal dysbiosis or BV is more alkaline than in healthy subjects. Healthy, fertile women present with a pH of about 3 to 5.5 (more specifically between pH 3.5 and 4.5) in the vagina, primarily as a result of lactic acid production. The increase of vaginal pH above 4.0-4.5 is detrimental for the survival of Lactobacillus bacteria. In some embodiments, the vaginal mucosa of subjects having vaginal dysbiosis or BV is greater than pH 4.5, e.g., pH 4.6, 4.7, 4.8, 4.9, 5.0, 5.1, 5.2, 5.3, or greater and is adjusted to predetermined levels by administration with the vaginal composition provided herein. The method of obtaining the vaginal composition may comprise a step of adjusting the pH of the preparation and or the vaginal niche to a predetermined level, wherein the predetermined level is pH 3.0-4.5, optionally pH 3.5-4.0. In embodiments, the pH is adjusted to 3.0-4.5, 3.2-4.3, 3.4-4.3, 3.5-4.0, or about 3.7-4.0. In embodiments, the predetermined level is approximately pH 3.5-4.0. In some embodiments, the pH is adjusted to the predetermined pH level by addition of acids, such as lactic acid according to routine methods in the art.

Predetermined level of viscosity: Cervicovaginal fluid is a viscous fluid, wherein the level of viscosity is variable and depends, inter alia, on the rate of mucociliary transport, the water content and the amount of glycoproteins or mucins present in the fluid. The viscosity of a fluid can be determined with any known methods in the art, such as a rheometer or viscosimeter. The method of obtaining the vaginal composition may comprise a step of adjusting the viscosity to a predetermined level. This enables that the vaginal composition can be aliquoted or stored in uniform and homogenous units. In some embodiments, the method of obtaining a vaginal composition comprises a step of adjusting the viscosity to a predetermined level of <1000 cP, <750 cP, or <500 cP. In some embodiments, the predetermined level is approximately 1-500 cP, 1-400 cP, 1-300 cP, 1-200 cP, 1-100 cP, 1-50 cP, or 1-25 cP. In some embodiments, the predetermined level is approximately 1-100 cP or 1-50 cP. Predetermined level of osmolarity: Adjusting the osmolarity of the vaginal composition ensures that the osmolarity and thus fluid homeostasis in the urogenital or vaginal tract is not perturbed. In embodiments, the method of obtaining the vaginal composition may comprise a step of adjusting the osmolarity to a predetermined level. In embodiments, the predetermined level of osmolarity of the vaginal composition is set to 200-500 mOsm/kg, such as 220-480 mOsm/kg, 250-450 mOsm/kg, 300-400 mOsm/kg, or 320-380 mOsm/kg. Osmolarity may be determined by methods known in the art, e.g., using a vapor pressure osmometer; and adjusted by addition of e.g., water to reduce osmolarity, or by addition of an excipient, such as mannitol, to increase osmolarity.

### EXAMPLES

### Example 1: Screening of Women to Identify Donors and Recipients

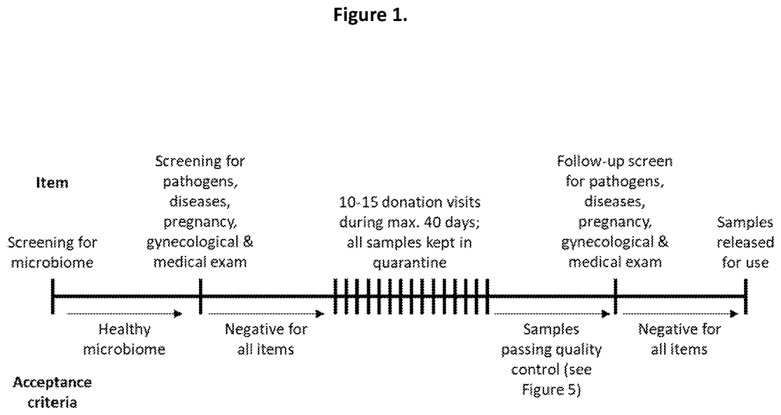

This Example describes the process of identifying suitable donors based on the analysis of the vaginal microbiome composition and absence of pathogens. A schematic overview of the screening is shown in FIG. 1.

### Participant Enrollment and Vaginal Samples

Healthy women without a history of vaginal health issues were recruited. Enrolling participants were informed, consented and provided a vaginal swab for microbiome analysis, and a separate swab for HPV analysis. A total of 96 women consented and provided samples. For the last 60 women screened, instead of a vaginal swab, a cervicovaginal secretion (CVS) sample was obtained using a vaginal self-sampling device (e.g., a pliable menstrual cup that is inserted into the vaginal cavity, such as a Softdisc menstrual cup by The Flex Company, also sold under the name ‘Flex disc’) to enable both microbiome and biomarker analysis, as well as a dedicated swab for HPV screening. Both sample types were obtained via self-sampling by donors after thorough instruction of the donor on the correct sampling procedure.

### Microbiome Analysis

The microbiome composition from the vaginal swab or CVS samples was determined by shotgun DNA sequencing analysis. Swabs for vaginal microbiome analysis were kept at 4° C. for up to 48 hours prior to DNA extraction. CVS samples were diluted with saline to reduce the viscosity, aliquoted (for procedure, see Example 3) and stored at −80° C. prior to DNA extraction. DNA was extracted from both sample types using the Molysis Complete5 kit (MolZym), which uses a differential lysis method to extract microbial DNA and remove human DNA. Shotgun sequencing and bioinformatics analyses of the microbiomes was performed by Seqbiome (Cork, IE). All samples were prepared for shotgun metagenomic sequencing according to Illumina Nextera XT library preparation kit guidelines, with the use of unique dual indexes for multiplexing with the Nextera XT index kit (Illumina). Samples were sequenced on an Illumina NextSeq 500 sequencing platform with a v2.5 kit (a 300-cycle kit/150 bp PE sequencing), at the Teagasc DNA Sequencing Facility, using standard Illumina sequencing protocols. Quality of the raw sequencing data was assessed using FastQC. Samples that did not meet minimum quality check cutoff or a sequencing depth of at least 4M reads underwent re-sequencing. Shotgun metagenomic sequencing data were then processed through analysis workflow that utilizes Kneaddata wrapper tool. Quality filtering and host genome decontamination (human) were performed utilizing Trimmomatic and Bowtie2. Taxonomic classification of quality filtered reads was further performed using Kraken2 species classifier using a customized version of Genome Taxonomy Database (GTDB) that also includes reference sequences belonging to archaea, fungi and viral genomes. Kraken2 classifications were then passed to Bracken tool to estimate species level abundance. The vaginal microbiome composition was determined for each putative donor and recipient by obtaining information regarding the presence and relative abundance of bacterial species.

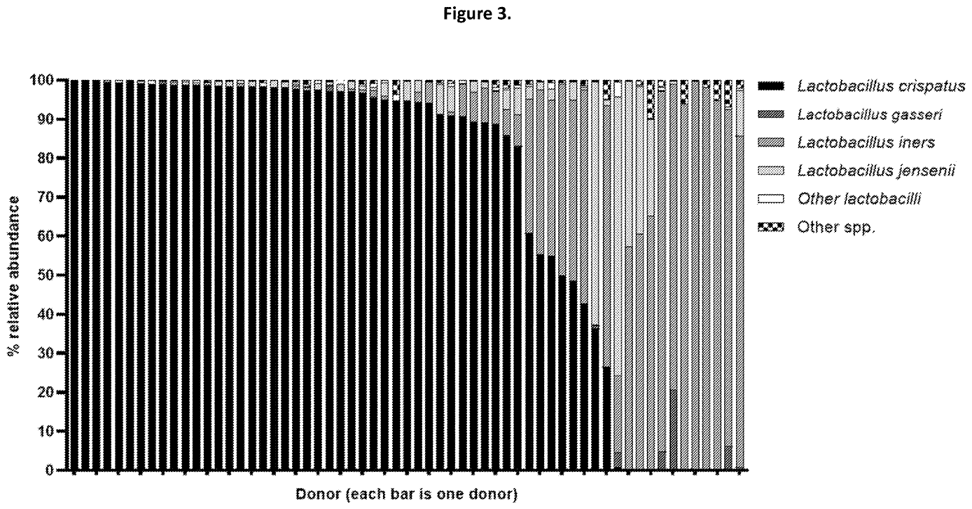

For this study, to qualify as a donor, the vaginal microbiome composition had to pass two criteria: to comprise at least 80% vaginal lactobacilli (L. crispatus, L. iners, L. jensenii, L. gasseri) and to comprise less than 5% selected pathogens (Atopobium spp., Prevotella spp., B. vaginale, and F. vaginae). The vaginal microbiome composition of 61 out of 96 women (64%) satisfied these two criteria and were considered healthy vaginal microbiomes. The relative abundance of the lactobacilli present in the vaginal microbiota are depicted in FIG. 3, wherein each bar represents the microbiome composition of a single healthy donor. The relative quantities of species are indicated in the y-axis. The microbiomes of the screened donors differed in their relative composition. In several instances, the microbiome was dominated by a single species, e.g., L. crispatus, wherein other microbiomes showed a heterogenous population of lactobacilli, e.g., comprising a combination of L. crispatus, L. iners and L. jensenii. Of note, even minute relative quantities, such as less than 0.05% could be detected (see, Table 2).

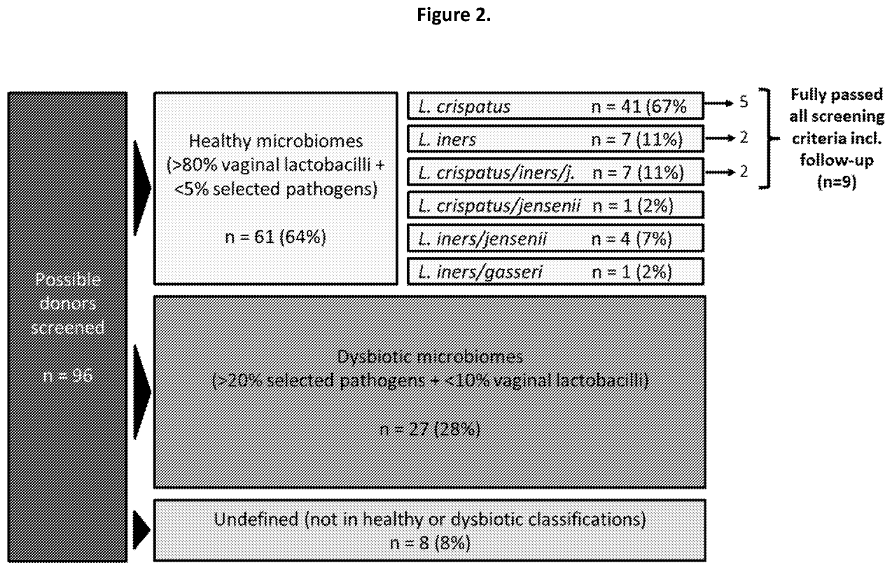

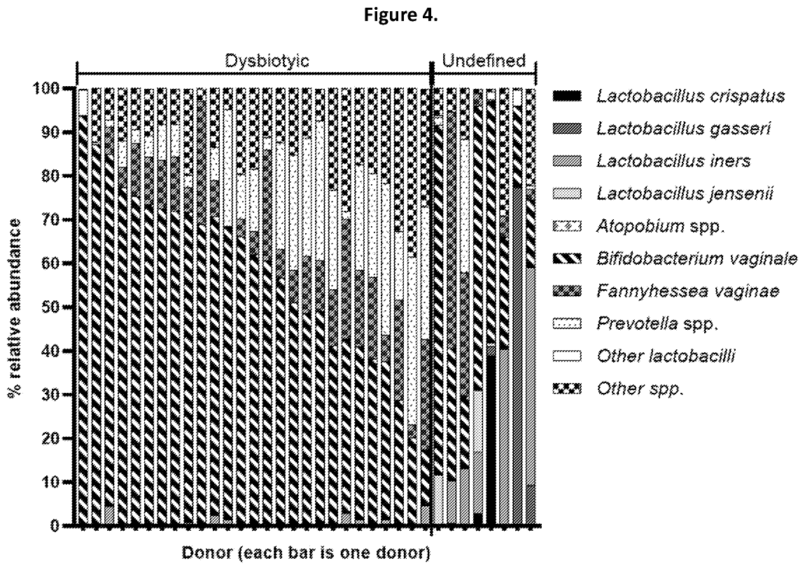

Of the 96 women screened, 28% (n=27) exhibited a dysbiotic microbiome (defined as >20% selected pathogens and <10% vaginal lactobacilli) (see, FIGS. 2 and 4). The women that exhibited dysbiotic vaginal microbiota were free of vaginal disease symptoms and of general good health. The remaining 8% (n=8) fell between the healthy and dysbiotic microbiome classifications, did not exhibit any vaginal disease symptoms, or contained species not classified/defined in the screening criteria (FIG. 4). These women did not qualify as putative donors or recipients.

The microbiome compositions of the healthy cohort (n=61) had a median vaginal lactobacilli abundance of 99.26% and selected pathogens of 0.03%. In contrast, the dysbiotic cohort (n=27) was characterized by a median vaginal lactobacilli abundance of 0.34% and selected pathogens of 87.21%. The undefined cohort (n=8) was characterized by a median vaginal Lactobacilli abundance of 35.76% and selected pathogens of 61.83%.

This suggests that the selection criteria (see, FIG. 2) can reliably distinguish healthy and dysbiotic vaginal microbiota with high certainty. Only healthy vaginal microbiomes were considered as putative donors and underwent further stringent screening procedures.

### HPV Screening

Human papillomavirus (HPV) is a highly prevalent viral infection, several strains of which are associated with cervical cancer. The prevalence of HPV-positive women is high. On the vaginal swabs taken for HPV screening during the donor's screening visit, DNA extraction was performed using the Qiagen QIAmp DNA mini kit. Extracted DNA from each donor sample was used in the SeeGene Anyplex II HPV28 kit and ran semi-quantitatively on the BioRad CFX96 Dx qPCR machine, which was set up and calibrated for this assay (Triolab, DK). The semi-quantitative method provides three levels of quantity for 28 different genotypes of HPV, which included all known high-risk genotypes. Only samples that were HPV-negative were considered suitable putative donors.

### Pathogen Check and Examination by Medical Staff

To minimize any adverse effects for the recipients, putative donors (women who had a healthy microbiome and passed the HPV screen) were further screened by a gynecologist for the presence of pathogens, pathobionts, and sexually transmitted diseases, and underwent a medical and gynecological examination to assess the presence of other diseases such as cancer or endometriosis. The procedure included an additional HPV test.

Standard diagnostic tests were performed for HV, Hepatitis A, B, and C, cytomegalovirus, Treponema, urinary tract infections, HPV, chlamydia, gonorrhea, Trichomonas, Herpes genitalis, Candida, Mycoplasma, and Streptococcus A, B, C, and G. During the medical examination, putative donors were subjected to a general health check including medical history and medication usage, demographics, heart rate and blood pressure measurements.

For approved donors, the same tests were again performed at a follow-up visit after they had provided their last donations over a specified time period (see, FIG. 1). Until approved donors passed the test at the follow-up visit, and all samples passed all quality checks (see, Example 4), the samples remained quarantined and not released for administration to a recipient.

### Donor Selection

Donors were selected on the screening procedure described above, including of microbiome analysis, pathogen screening, and medical and gynecological examination. Only subjects that had a healthy vaginal microbiome, had no positive pathogen test, and had no abnormal findings in the medical and gynecological exam were considered suitable donors and were enrolled in the program (see also, Example 2).

Following the screening protocol, 12 (13%) out of 96 screened women were enrolled into the donor program and started their donation visits. Of these, three failed to provide sufficient donations or failed during follow-up screening. Overall, of all 96 women who enrolled for screening 900 fully passed all screening criteria resulting in nine approved donors (n=9) (Table 1).

Table 1 provides an overview of the pathogens and pathobionts that were screened for in donor subjects (n=38) that had passed HPV screening.

TABLE 1

| | n positive |
|---|---|
| Screening | out of n = 38 |
| HIV | 0 |
| Hepatitis A/B/C | 0 |
| CMV IgM | 0 |
| Treponema | 0 |
| UTI | 2 |
| HPV | 13 |
| Chlamydia | 1 |
| Gonorrhoeae | 0 |
| Trichomonas | 0 |
| Herpes genitalis | 0 |
| Mycoplasma | 1 |
| Strep A/B/C/G | 4 |
| Candida | 7 |
| Other (e.g., abnormal gynecological findings) | 4 |
| Total passed - out of the original 96 women | 12 |
| Failed during donations or at follow-up | 3 |
| Total fully passed/approved donors | 9 |

The composition of the Vaginal composition of all screened subjects (n=96) including the fully approved donors (n=9) is summarized in Table 2.

Table 2 Vaginal microbiota compositions of four cohorts: (i) Healthy, (ii) fully approved donors, (iii) dysbiotic and (iv) undefined. Shown are the relative amounts of the quantified bacteria in median and interquartile range (IQR) (in %) relative to the total amount of detectable species in the sample preparation.

TABLE 2

| | Healthy | | Fully approved | | Dysbiotic | | Undefined | |
|---|---|---|---|---|---|---|---|---|
| | (n = 61) | | donors (n = 9) | | (n = 27) | | (n = 8) | |
| Species | median | IQR | median | IQR | median | IQR | median | IQR |
| Lactobacillus | 94.75 | 50.05 | 97.42 | 50.16 | 0.00 | 0.00 | 0.03 | 1.08 |
| crispatus | | | | | | | | |
| Lactobacillus gasseri | 0.00 | 0.08 | 0.00 | 0.00 | 0.00 | 0.00 | 0.28 | 3.84 |
| Lactobacillus iners | 0.84 | 42.20 | 0.84 | 46.48 | 0.34 | 1.11 | 11.47 | 20.79 |
| Lactobacillus jensenii | 0.74 | 2.82 | 0.07 | 1.34 | 0.00 | 0.00 | 0.10 | 3.42 |
| Total vaginal | 99.26 | 1.44 | 99.62 | 0.37 | 0.34 | 1.10 | 35.76 | 33.35 |
| lactobacilli | | | | | | | | |
| Atopobium spp. | 0.00 | 0.00 | 0.00 | 0.00 | 0.00 | 0.01 | 0.00 | 0.00 |
| Bifidobacterium | 0.01 | 0.30 | 0.00 | 0.08 | 61.60 | 31.71 | 27.98 | 39.65 |
| vaginale | | | | | | | | |
| Fannyhessea vaginae | 0.00 | 0.00 | 0.00 | 0.00 | 10.70 | 9.75 | 2.63 | 10.11 |
| Prevotella spp. | 0.00 | 0.01 | 0.00 | 0.00 | 10.23 | 22.19 | 0.02 | 0.46 |
| Total selected | 0.03 | 0.50 | 0.01 | 0.08 | 87.21 | 10.10 | 61.83 | 49.59 |
| pathogens | | | | | | | | |
| Other lactobacilli | 0.31 | 0.55 | 0.15 | 0.14 | 0.00 | 0.00 | 0.28 | 0.98 |
| Other species | 0.14 | 0.58 | 0.03 | 0.33 | 12.48 | 10.96 | 5.71 | 13.50 |

“Healthy”, “Dysbiotic” and “Undefined” cohorts were categorized based on the relative quantities of the vaginal lactobacilli and pathogens as described above (see also, FIG. 2). “Fully approved donors” refers to subjects that, in addition to passing the first screen successfully underwent additional screens, e.g., to exclude STIs and other infections and medical conditions as described above. Subjects that passed all screening criteria and qualified as suitable donors (n=9) had a median vaginal lactobacilli concentration of 99.62% and selected pathogens of 0.01%. Approved donors had the highest amount of selected vaginal lactobacilli and the lowest amount of vaginal pathogens of the groups. Fully approved donors were not limited to L. crispatus-dominant vaginal microbiota compositions but comprised a mixture of lactobacilli species or were L. iners dominant.

### Recipient Selection

Recipients were recruited and enrolled in clinical studies. In one study, healthy volunteer women aged 18 to 45 were recruited for screening and participation. Only women without vaginal disease symptoms and of general good health were enrolled for screening. Another study enrolled a single patient with vaginal symptoms. All recipients were classified as having vaginal dysbiosis (harboring a dysbiotic vaginal microbiome) according to the inclusion criteria defined above (see, FIG. 2): >20% selected pathogens (Atopobium spp., Prevotella spp., B. vaginale, and F. vaginae) and <10% vaginal Lactobacilli.

### Example 2: Obtaining Donor CVS Samples

This Example describes the process of obtaining vaginal microbiota samples from a donor, wherein the sample comprises vaginal compositions that comprise substantially complete vaginal microbiota.

### Donation Visit Setup

Donors that successfully passed the screening process described in Example 1 were invited to donate cervicovaginal secretions (CVS) within a time period of 40 days between the two gynecologist/pathogen check visits (see, FIG. 1). Each donor provided 10-15 donations at any time during the 40 days except on days during menstruation and one day thereafter. The donations were spaced at least 16 hours apart.

During the donation period, donors needed to adhere to the following restrictions: abstinence from vaginal and anal sexual intercourse; no swimming in lakes, Jacuzzis and swimming pools; no use of intravaginal products (e.g., tampons, soap). At each visit, the donor filled out a questionnaire to affirm adherence to these restrictions together with general health questions.

After the donation period, the donors underwent a follow-up check at the gynecologist. Only if they passed this follow-up, were their samples considered for release and use for administration (see, FIG. 1, Example 1). Until that time, and until all samples passed all quality checks (see, Example 4), all samples were kept in quarantine.

### Donor Self-Collection of CVS

Cervicovaginal secretion (CVS) samples were obtained through self-collection using a vaginal self-sampling device after thorough instructions. Donors obtained CVS samples in a dedicated, hygienically designed donor room. The donor room was cleaned with 70% ethanol after each donor and subjected to biweekly environmental monitoring to check for Enterobacteriaceae, total aerobic microbial count, and yeast and mold. This setup maximizes cleanliness and minimizes minimized processing time, compared to, e.g., home sampling. Only a single contamination was detected in one of the donor samples consisting of skin bacteria, caused by the donor not following the sampling procedure correctly.

The vaginal self-sampling device was a single use menstrual cup with a flexible/pliable ring and plastic foil cup (described in Example 1). For CVS donations, it was not worn like a menstrual cup over the cervix, but instead used as a large swab by inserting it partially folded into the vagina, leaving it in a longitudinal position for about 10 seconds and twisting it along its longitudinal axis while removing it. The donor deposited the vaginal self-sampling device into a provided labeled and pre-weighed sterile 50 mL tube. After 15-20 min, this process was repeated a second time with a new vaginal self-sampling device, which was placed in a separate sterile 50 mL tube.

The soft and pliable material of the vaginal self-sampling device in conjunction with it being inserted partially folded into the vaginal canal enables the effective collection of CVS from the surface of the vaginal cavity, without damaging or irritating the vagina. The vaginal self-sampling devices are intended and safe for vaginal use, but to ensure maximum donor safety and control, each batch of the devices was screened for contamination of Enterobacteriaceae and confirmed to be free of any contamination.

The procedure described here is believed to have several advantages over using the vaginal self-sampling device over a prolonged time period as a collecting device over the cervix. With the above method, the bacteria comprised in the CVS sample will spent less time in contact with the vaginal self-sampling device, and the amount of vaginal mucus and vaginal bacteria that is collected is increased. The vaginal epithelium contains the substrates for the vaginal microbiome.

Thus, a sample obtained in this manner has a higher concentration of viable vaginal lactobacilli and mucus, and a lower amount of fluid from the endometrium.

### Example 3: Producing a Vaginal Composition

This Example demonstrates the production of a vaginal composition from cervicovaginal secretions.

### Process to Produce a Vaginal Composition

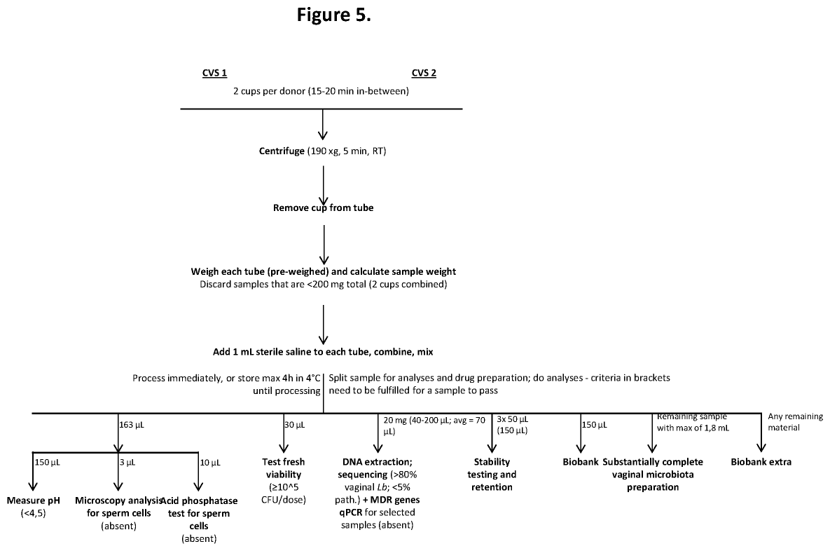

A schematic representation of the sample processing is provided in FIG. 5. At each visit, the donor was provided with two 50 mL sterile tubes, which were pre-labelled and pre-weighed, and two vaginal self-sampling devices, along with the questionnaire (see, Examples 1 and 2). The CVS was collected by centrifuging for 5 min at 190×g and ambient temperature. The low speed collects the CVS from the device, while not phase-separating into layers. All further steps were conducted under sterile conditions at ambient temperature. The self-sampling devices were discarded from the tubes and the CVS sample weight was determined. Samples with a total (two tubes combined) weight of less than 200 mg were discarded. Samples in which blood was visibly present were discarded.

The sample in each tube was mixed with 1 mL saline to reduce viscosity, after which the two samples were combined and aliquoted for storage and further testing. The two combined samples were then distributed over several aliquots for quality control (see, Example 4) and storage as shown in FIG. 5.

The cryovials containing samples for storage were placed in a CoolCell (Corning) and then at −80° C. A CoolCell has a controlled cooling rate of 1° C. per minute, which ensures maintaining maximum viability of the samples. After a minimum of 2 hours, the samples were transferred into a regular −80° C. storage box labelled ‘quarantined’. Samples were released only after all release criteria had been met (see, FIG. 5 and as described above).

### Example 4: Quality Control

This Example describes the quality control of a vaginal composition produced from cervicovaginal secretions, so it can be used safely for administration.

### Overall Procedure

A schematic representation of the sample processing procedure with details about quality control is provided in FIG. 5. This procedure is highly optimized, in such a way that the volumes used for analyses and quality control are as low as possible to minimize loss of vaginal composition. The sample used for pH measurement is discarded after measurement. The retention vial is maintained for at least 1 year after administration for safety reasons (e.g., to check for STIs in case one occurred in a recipient). Every first and last sample of each donor was subjected to a qPCR analysis to check for the absence of multi-drug resistance genes (MDR genes).

### Analyses for Quality Control

The pH was measured using a micro-pH probe. Technical triplicate measurements were performed, and repeated if the difference between measurements was >0.2. The sample used for pH measurement is discarded after measuring. Microscopy was performed. The absence of sperm cells was determined optically by microscopy and using acid phosphate paper, a highly sensitive and selective method to identify sperm cells. Viability was tested by colony-forming units (CFU) count on de Man, Rogosa and Sharpe (MRS) agar plates or with viable cell count (VCC) for donors who at screening were dominant in L. crispatus, L. jensenii, or L. gasseri. For donors who at screening were dominant for L. iners, this method could not be used. Instead, viability for these samples was tested using BacLight live/dead staining (ThermoFisher Scientific) and counting the viable cells/mL in a Thoma cell counting chamber under the microscope. Within one week after a sample was used for administration, a stability vial was used to test cell viability at the time of vaginal administration, and dose calculation.

DNA extraction was performed using the Molysis Complete5 kit (Molzym) according to the manufacturer's instructions to obtain sufficient bacterial sequence reads to perform in silico engraftment check after administration based on metagenome data (see, Example 1 for methods used). The multi-drug resistance (MDR) marker qPCR was performed using the same DNA as was used for Shotgun sequencing, using the SeeGene Allplex Entero-DR qPCR assay kit on a BioRad CFX96 Dx qPCR machine calibrated for the assay. This kit allows single or multiple detection of carbapenemase genes (NDM, KPC, OXA-48, VIM, IMP), extended spectrum beta-lactamase (ESBL) genes (CTX-M), and vancomycin resistance genes (VanA, VanB).

### Example 5: Donor Microbiome Characterization

This Example describes the vaginal compositions suitable for vaginal administration.

### Microbiome Compositions During the Donation Period

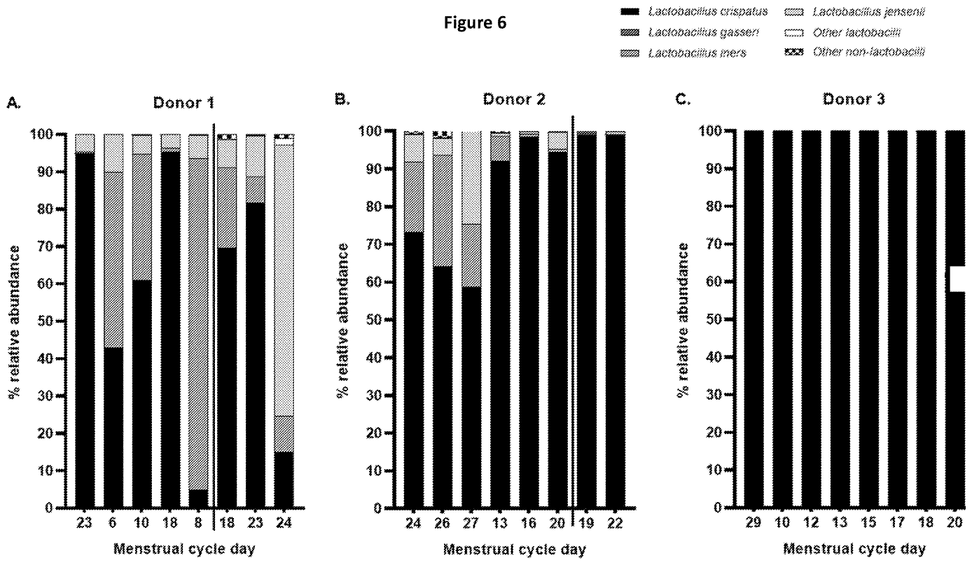

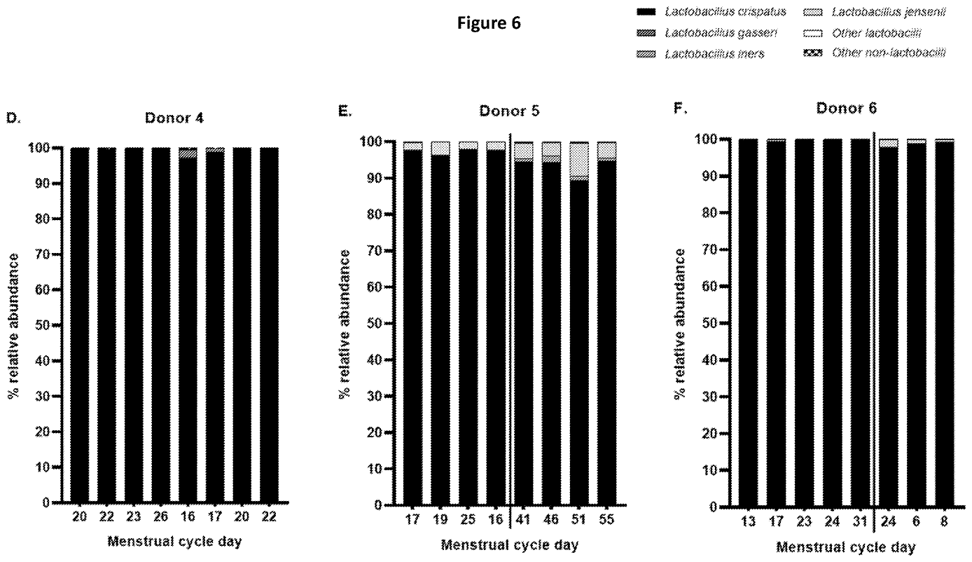

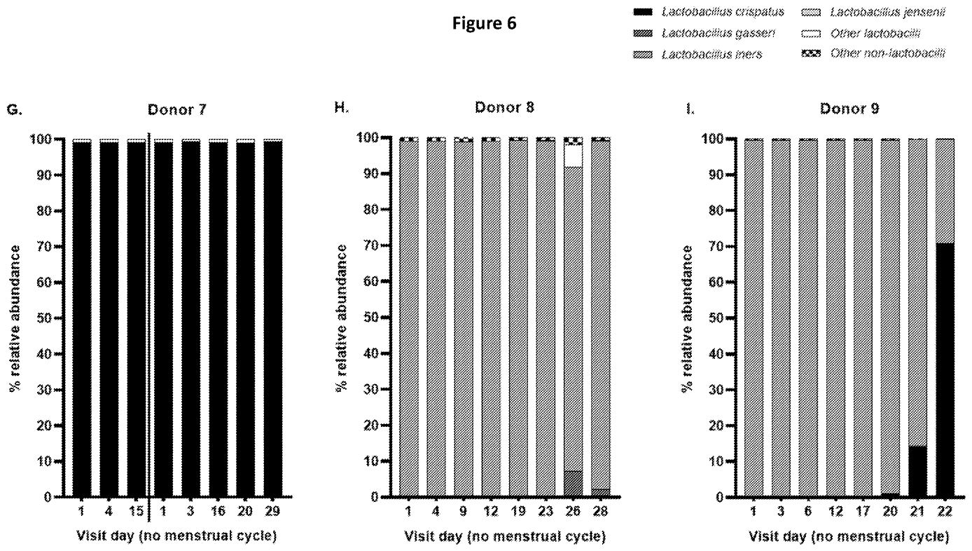

It is known that the vaginal microbiome in women can fluctuate over the menstrual cycle or depending on sexual activity (Gajer et al., 2012, Science Translational Medicine). For donors with mixed species compositions that did not use contraception (Table 3), fluctuations between the species were observed over the menstrual cycle (FIG. 6 A, B), but the totality of L. crispatus, L. iners, L. jensenii, L. gasseri in the sample remained stably >80%. The microbiome of donors with a strongly L. crispatus-dominant microbiome was very stable over extended periods of time (e.g., Donor 7, FIG. 6 G).

The microbiome of L. iners-dominant donors was mostly stable, although in one case a shift between L. iners and L. crispatus was observed towards the end of the donation period (FIG. 6 I). This donor did not have a menstrual cycle due to due contraception. The microbiome stability observed in the donors is likely aided by the restrictions that the donors adhere to, such as, e.g., no sexual intercourse.

Some donors returned for a second round of donation visits. In FIG. 6, this is indicated by a vertical line. There was a 1 month in-between two rounds for donors 2, 5, and 7, and a 1,5-months for donor 1. For donor 6, there was a 2-months in-between the rounds, and she changed from using contraceptive pills in the first round to no hormonal contraceptives in the second round.

In several donor samples, a certain species (mostly L. jensenii) was present in small but very consistent amounts (0.3-3.5%). In one of these donors, an even smaller amount of L. iners was consistently present (0.07-0.33%). Even smaller amounts of both L. iners (0.02-0.10%) and L. jensenii (0.01-0.03%) were consistently observed in yet another L. crispatus-dominant donor. In the two donors with mixed species composition, none of the species present in the mixture fully disappeared (below detection level) over the donation period even though their relative abundances sometimes decreased to as low as 0.27%. Altogether, this implies that the vaginal microbiome is a stable ecosystem and points to a symbiotic relationship between the different Lactobacillus species in one person. This is further substantiated by the observations of the vaginal administration of the preparations, as described in Example 6.

In addition to the four main vaginal lactobacilli (L. crispatus, L. iners, L. jensenii, L. gasseri), other lactobacilli were regularly observed to be consistently present in small amounts in donor samples (mostly <0.05%): L. acetotolerans, L. acidophilus, L. amylovorus, L. gallinarum, L. gigeriorum, L. helveticus, L. johnsonii, L. kefiranofaciens, L. kitasatonis, L. paragasseri, L. psittaci, Lactobacillus sp002911475, L. taiwanensis, L. ultunensis, L. coleohominis, L. reuteri, and L. vaginalis. The stable presence of these lactobacilli further points to a complex ecosystem that in its entirety might be needed to promote and maintain a healthy vaginal niche.

Some other lactobacilli appeared sporadically in a subset of the donor samples. L. helveticus was not observed in the two L. iners-dominant donors, and only appeared in the last visit of a single donor who shifted from L. iners-dominant to L. crispatus-dominant, which may suggest that certain species prefer cohabitation with each other while others might be incompatible in the ecosystem. In contrast to reports in the literature where L. iners-dominant donors were not considered suitable for administration, the data suggest that L. iners-dominant microbiomes of fully approved donors have a very stable and healthy ecosystem (see also inflammatory marker data in Example 7) and are suitable donors for vaginal compositions.

L. gasseri was infrequently observed as a dominant species (FIG. 3) but was typically found in small amounts in several of the donors (FIG. 6, Table 1). L. gasseri was detected in L. crispatus-dominant donor 4 (FIG. 6 D) in relative concentrations of 0.01-2.23%; in mixed microbiome donor 1 (FIG. 6 A) in relative concentrations of 0.01-0.07%; and in L. iners-dominant donor 8 (FIG. 6 H) in relative concentrations of 0.3-70.14%. The consistent maintenance of minor Lactobacillus species suggests that the minor species might play a hitherto unknown role in maintenance or stability of the vaginal ecosystem.

### Physical Properties of the Obtained Samples

The weight of samples fluctuated among donations of the same donor and among donors, but on average was >200 mg. CFU and pH remained stable and within the defined threshold of <4.5 (Table 3). Altogether, this stability over both the microbiome composition and sample properties indicates the health and stability of the donor microbiomes.

Table 3 summarizes the properties of the CVS and vaginal compositions that are generated. Each row contains data for one donor during their 10-15 donations over a 40-day period. The ‘Time window’ column lists the number of days during which the donor provided the number of samples listed in the ‘Nr of donations’ column. Donor ID is the same as in the microbiome graphs in FIG. 6. The weight is the combined weight of the two samples provided by the donor, prior to adding saline. The dose is the final amount of mL in the vaginal composition vial used for dosing a recipient, after all volumes for analyses have been taken out (see, FIG. 5). The maximum dose is 1.8 mL to fit in a cryovial. Viable cells/dose was determined by CFU plate counting on MRS medium grown anaerobically for 2-3 days for all species except for the two L. iners-dominant donors (8 and 9). For these, viable cells/dose was determined by live/dead staining and counting viable cells in a counting chamber under the microscope. Abbreviations: hIUD: hormonal intrauterine device; pill: oral contraceptive.

TABLE 3

| | | | | Time | Ranges (min-max) | | | | Averages | | | |
|---|---|---|---|---|---|---|---|---|---|---|---|---|
| Donor | | Contra- | No of | window | Weight | Dose | | Viable | Weight | Dose | | Viable |
| ID | Microbiome | ception | donations | (days) | (mg) | (mL) | pH | cells/dose | (mg) | (mL) | pH | cells/dose |
| 1 | Mixed | none | 15 | 28 | 244-1035 | 1.5-1.8 | 3.9-4.4 | 8.6E+06- | 515 | 1.6 | 4.1 | 1.2E+08 |
| | | | | | | | | 3.1E+08 | | | | |
| 2 | Mixed | none | 10 | 36 | 311-918 | 1.4-1.8 | 4.1-4.3 | 1.9E+08- | 597 | 1.7 | 4.2 | 3.6E+08 |
| | | | | | | | | 5.4E+08 | | | | |
| 3 | L. crispatus | none | 11 | 25 | 628-1778 | 1.7-1.8 | 3.7-3.8 | 5.1E+07- | 1206 | 1.8 | 3.8 | 1.5E+10 |
| | | | | | | | | 8.4E+10 | | | | |
| 4 | L. crispatus | hIUD | 14 | 31 | 195-486 | 1.3-1.7 | 3.9-4.2 | 7.2E+07- | 334 | 1.5 | 4.1 | 1.9E+08 |
| | (+ some L. gasseri) | | | | | | | 3.4E+08 | | | | |
| 5 | L. crispatus + | pill | 12 | 30 | 340-823 | 1.5-1.8 | 3.7-4.0 | 3.3E+08- | 525 | 1.7 | 3.9 | 7.4E+09 |
| | L. jensenii | | | | | | | 1.0E+11 | | | | |
| 6 | L. crispatus + | pill | 14 | 20 | 276-703 | 1.3-1.8 | 3.8-3.9 | 5.0E+07- | 487 | 1.6 | 3.8 | 1.7E+10 |
| | L. jensenii | | | | | | | 1.0E+11 | | | | |
| 7 | L. crispatus + | pill | 13 | 16 | 376-710 | 1.6-1.8 | 3.8-4.0 | 3.6E+08- | 542 | 1.7 | 3.9 | 8.5E+09 |
| | other lactobacilli | | | | | | | 1.0E+11 | | | | |
| 8 | L. iners | hIUD | 12 | 31 | 532-1593 | 1.6-1.8 | 3.9-4.0 | 4.3E+08- | 868 | 1.8 | 3.9 | 8.5E+08 |
| | (+ L. gasseri towards | | | | | | | 1.2E+09 | | | | |
| | end) | | | | | | | | | | | |
| 9 | L. iners | hIUD | 14 | 24 | 233-627 | 1.4-1.8 | 3.9-4.2 | 1.3E+08- | 446 | 1.6 | 4.0 | 3.0E+08 |
| | (+ L. crispatus | | | | | | | 6.0E+08 | | | | |
| | towards end) | | | | | | | | | | | |
| Average | | | 13 | 27 | | | | | 614 | 1.6 | 4.0 | 5.5E+09 |

It was further evaluated whether repeated CVS donations would affect the composition of the sample. Donors provided two samples 2× per day with a 6-hour interval and 3× per day with a 4-hour interval. Both of the repeated CVS donation procedures resulted in a significant increase of the pH (approx. 0.4 increase), and a decrease of sample weight (to around or below 200 mg) and CFU (1 log), rendering repeated sampling within a single day unsuitable. This suggests that the microbiome requires more than 6 hours to fully regenerate after sampling. Consequently, donors were allowed to give samples once a day at most with approximately 16-hour intervals between donations.

### Example 6: Treating Dysbiotic Microbiomes Through Vaginal Administration of the Preparation

This Example demonstrates how a vaginal composition can be used to revert a recipient's dysbiotic microbiome to a donor's healthy microbiome using vaginal administration, thereby treating the dysbiotic microbiome.

### Administration of Vaginal Compositions and Results

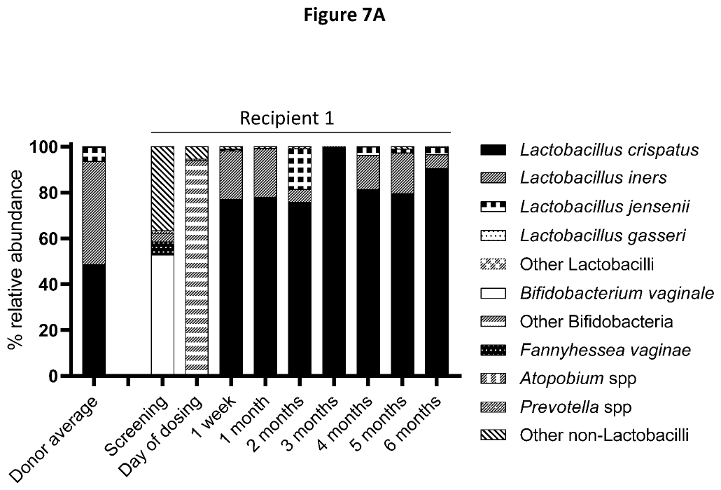

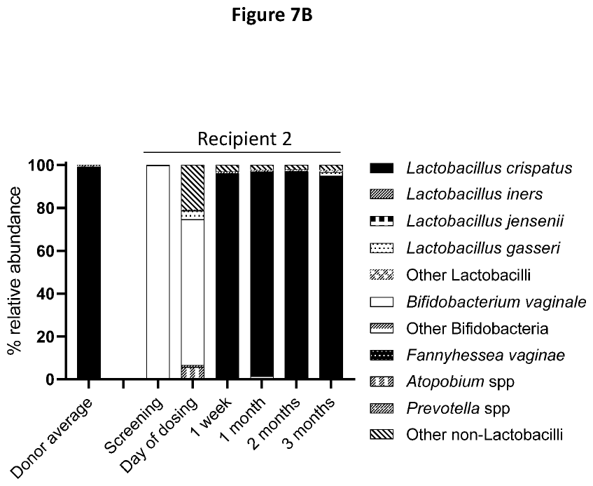

The vaginal composition sample was first thawed by moving it from −80° C. to room temperature for 40 minutes. Thereafter, the sample was placed in an incubator at 37° C. for 30 minutes. The tube containing the sample was then gently inverted 5 times for mixing. The sample was drawn up into a 5 mL syringe and applied intravaginally to the recipient lying in the lithotomy position using an insemination catheter attached to the 5 mL syringe. The recipient was instructed to remain lying down in a horizontal position for 30 minutes following the sample being inserted intravaginally. 21 recipient subjects were treated with the vaginal composition obtained from healthy donors as described above. Two representative changes of a dysbiotic vaginal microbiota after administration of a vaginal composition are shown in FIG. 7 and discussed in detail in the following. Both recipients received 3 doses (1 dose/day over the course of 3 consecutive days) (FIGS. 7A and 7B). The recipients were not pre-treated with antibiotics prior to or during the administration of the preparation. After administration of the preparation, the microbiome changes of the recipients demonstrate that the vaginal composition is capable of changing a dysbiotic microbiome to a microbiome more closely resembling that of the donor, by administration of the vaginal composition alone (i.e., without treatment with antibiotics).

All recipients were enrolled based on their microbiome at screening, which was generally one month before their visit. At their visit for administration of the vaginal composition, their microbiome was checked again as baseline measurement. Both recipients were vaginally asymptomatic. Recipient 1 was vaginally asymptomatic, while recipient 2 showed vaginal disease symptoms at the time of administration. Recipient 2 reported a marked decrease in symptoms 1 week after administration of the preparation, and full disappearance of all vaginal disease symptoms after 2 weeks.

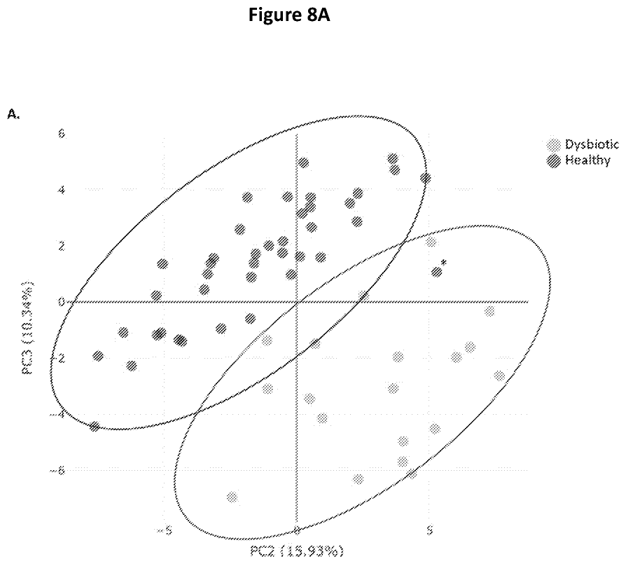

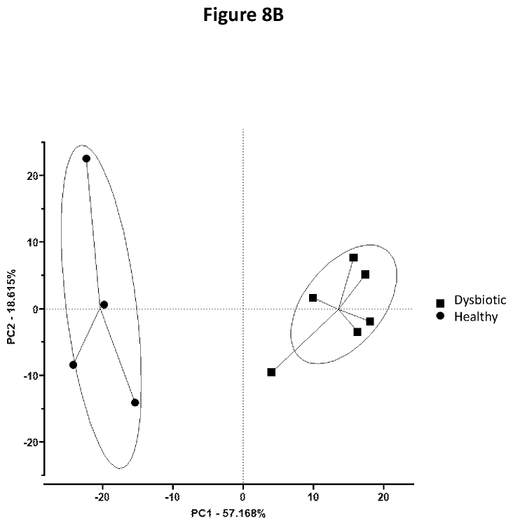

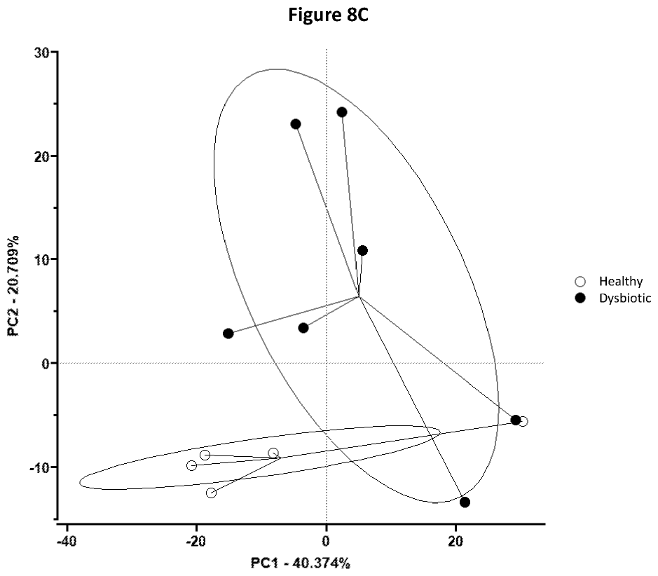

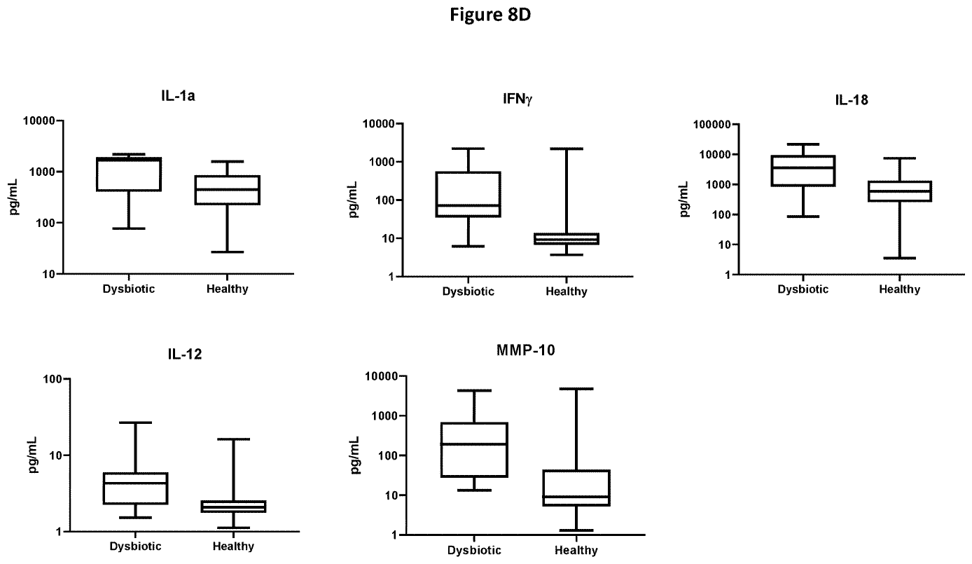

The post-administration microbiome of the recipients appeared similar to the donors', in what could be termed ‘microbiome twinning’. The dysbiotic vaginal microbiomes of recipients 1 and 2 were thus successfully reversed to a healthy, non-dysbiotic microbiome, and remained stable over a period of at least 5 or 3 months, respectively (FIGS. 7A and 7B). Overall, the post-administration recipients clustered together in an unsupervised principal component analysis (PCA) plot with the donor samples, while the pre-administration recipients form a separate cluster (FIG. 8A-8C). This suggests that the vaginal microbiome of the recipient after administration of the preparation did not only change from a dysbiotic state but resembles the vaginal microbiome composition of the healthy, non-dysbiotic donor. As a blinded approach, to check engraftment by the specific donor microbiome in one of the recipients vaginal niche, the post-treatment recipient microbiome was compared to a library of all donor microbiomes (metagenomic sequencing data).

It was possible to identify correct donor sample from the donor library based on the closeness to the recipient microbiome, suggesting that engraftment of the donor microbiome in the recipient was successful.

The microbiome compositions of the recipients pre- and post-administration of the vaginal composition(s), and the composition of the respective donor are summarized in Table 4. The Table shows the relative abundances (determined by Shotgun sequencing) of species in donor and recipient samples, wherein after administration of the preparation ‘microbiome twining’ was observed in each case, i.e. donor species cultivated in the vaginal niche of recipients including species that were present in the donor microbiome in low quantities, and were absent in the recipient prior to administration of the preparation. The relevant species where this phenomenon is observed are highlighted in the grey columns.

The post-administration microbiome of the recipient included species that were present in minor quantities in the donor microbiome (Table 4; wherein L. is Lactobacillus and B. is Bifidobacterium), suggesting that the entire stable ecosystem was transferred from the donor to the recipient. It was also observed that these species were stably present for all donation visits of most donors (see, Example 5). The transfer of the entire ecosystem to the recipient through administration of a vaginal composition may allow for successful engraftment and colonization of the lactobacillus-dominated donor microbiome, even in the absence of an antibiotic pre-treatment used to eradicate residing pathogens and pathobionts and create a substantially empty microbial niche. This might explain why single species or single strain preparations that have previously been tested generally do not work well. Alternatively or in addition, there might be a need for certain substances in the vaginal composition (e.g., mucin, lactic acid, etc.) to support initial engraftment.

Two different donors were used in recipient 1 and recipient 2, with three multiple visits for each.

For each recipient, across those three samples, the species are the same but their relative abundance is different, indicating that the abundance of a species is not necessarily the determining factor.

Engraftment appears successful when the entire ecosystem is present in the donor material, thus requiring a vaginal composition.

Other researchers have previously suggested a harmful role of L. iners. However, L. iners is present in the donor microbiomes that were tested. In women that pass the stringent screening criteria described herein, L. iners may also play a be beneficial role in representing healthy, stable microbiomes. This confirms the selection process to use all four main Lactobacillus species for administration of the preparation.

This Example thus demonstrates the successful treatment of a dysbiotic vaginal microbiome by administration of a vaginal composition to a dysbiotic recipient, wherein post-administration the recipient's microbiota composition closely resembled the donor's microbiota composition. While antibiotic treatment prior to administration was thought to be required—as proposed by others—these data demonstrate that pretreatment with antibiotics is not required if vaginal compositions are provided that contain a substantially complete ecosystem.

TABLE 4
Relative abundance (%) of vaginal Lactobacillus species and selected vaginal pathogens
after administration of the vaginal composition in two exemplary recipients.

| | L. | L. | | L. | | Limasilactobacillus | Other |
|---|---|---|---|---|---|---|---|
| Recipient 1 | crispatus | gasseri | L. iners | jensenii | L. helveticus | vaginalis | Lactobacilli |
| Dose 1 Donor | 42.84 | 0.00 | 47.17 | 9.84 | 0.01 | 0.02 | 0.01 |
| Dose 2 Donor | 46.65 | 0.00 | 48.30 | 4.89 | 0.00 | 0.05 | 0.00 |
| Dose 3 Donor | 56.07 | 0.00 | 39.78 | 3.83 | 0.00 | 0.10 | 0.00 |
| Screening | 0.02 | 0.00 | 3.63 | 0.00 | 0.00 | 0.00 | 0.00 |
| Day of dosing | 0.08 | 0.00 | 0.02 | 0.00 | 0.00 | 0.00 | 0.00 |
| 1 week | 77.05 | 0.00 | 21.17 | 0.50 | 0.02 | 0.02 | 0.00 |
| 1 month | 77.85 | 0.00 | 21.28 | 0.08 | 0.02 | 0.11 | 0.01 |
| 2 months | 75.75 | 0.02 | 5.65 | 17.75 | 0.02 | 0.08 | 0.05 |
| 3 months | 99.39 | 0.00 | 0.01 | 0.41 | 0.02 | 0.02 | 0.01 |
| 4 months | 81.31 | 0.00 | 14.89 | 3.57 | 0.01 | 0.11 | 0.00 |
| 5 months | 79.49 | 0.00 | 17.72 | 1.65 | 0.01 | 0.62 | 0.01 |
| 6 months | 90.36 | 0.01 | 6.02 | 3.24 | 0.01 | 0.06 | 0.01 |

| | | | Other | | | |
|---|---|---|---|---|---|---|
| | Atopobium | | Bifido- | Fannyhessea | Prevotella | Other non- |
| Recipient 1 | spp | B. vaginale | bacteria | vaginae | spp | Lactobacilli |
| Dose 1 Donor | 0.00 | 0.00 | 0.00 | 0.00 | 0.00 | 0.11 |
| Dose 2 Donor | 0.00 | 0.00 | 0.00 | 0.00 | 0.00 | 0.10 |
| Dose 3 Donor | 0.00 | 0.00 | 0.00 | 0.00 | 0.00 | 0.21 |
| Screening | 0.03 | 52.64 | 0.03 | 5.75 | 1.29 | 36.62 |
| Day of dosing | 0.07 | 0.04 | 93.88 | 0.00 | 0.55 | 5.36 |
| 1 week | 0.00 | 0.00 | 0.00 | 0.00 | 0.31 | 0.92 |
| 1 month | 0.00 | 0.03 | 0.00 | 0.00 | 0.01 | 0.59 |
| 2 months | 0.01 | 0.01 | 0.02 | 0.00 | 0.00 | 0.65 |
| 3 months | 0.00 | 0.06 | 0.00 | 0.01 | 0.00 | 0.06 |
| 4 months | 0.00 | 0.04 | 0.00 | 0.00 | 0.00 | 0.06 |
| 5 months | 0.00 | 0.13 | 0.00 | 0.02 | 0.03 | 0.31 |
| 6 months | 0.00 | 0.07 | 0.00 | 0.01 | 0.00 | 0.21 |

| | L. | L. | L. | L. | L. | L. | L. | Limasilactobacillus | Limasilactobacillus |
|---|---|---|---|---|---|---|---|---|---|
| Recipient 2 | crispatus | gasseri | iners | jensenii | amylovorus | helveticus | johnsonii | reuteri | vaginalis |
| Dose 1 Donor | 99.10 | 0.00 | 0.00 | 0.00 | 0.52 | 0.04 | 0.10 | 0.13 | 0.00 |
| Dose 2 Donor | 99.18 | 0.00 | 0.00 | 0.00 | 0.44 | 0.04 | 0.10 | 0.11 | 0.00 |
| Dose 3 Donor | 99.25 | 0.00 | 0.00 | 0.00 | 0.41 | 0.04 | 0.09 | 0.10 | 0.00 |
| Screening | 0.00 | 0.06 | 0.03 | 0.01 | 0.00 | 0.00 | 0.00 | 0.00 | 0.01 |
| Day of dosing | 0.00 | 4.09 | 0.00 | 0.01 | 0.00 | 0.00 | 0.04 | 0.00 | 0.01 |
| 1 week | 95.74 | 0.25 | 0.00 | 0.00 | 0.39 | 0.03 | 0.11 | 0.11 | 0.00 |
| 1 month | 95.39 | 0.25 | 0.00 | 0.00 | 0.46 | 0.04 | 0.11 | 0.12 | 0.01 |
| 2 months | 96.52 | 0.10 | 0.01 | 0.00 | 0.52 | 0.03 | 0.11 | 0.12 | 0.00 |
| 3 months | 94.22 | 1.56 | 0.00 | 0.00 | 0.67 | 0.03 | 0.14 | 0.16 | 0.00 |

| | | | | | | | Other non- |
|---|---|---|---|---|---|---|---|
| | Other | Atopobium | B. | Other Bifido- | Fannyhessea | Prevotella | Lacto- |
| Recipient 2 | Lactobacilli | spp | vaginale | bacteria | vaginae | spp | bacilli |
| Dose 1 Donor | 0.11 | 0.00 | 0.00 | 0.00 | 0.00 | 0.00 | 0.00 |
| Dose 2 Donor | 0.09 | 0.00 | 0.00 | 0.00 | 0.00 | 0.00 | 0.04 |
| Dose 3 Donor | 0.10 | 0.00 | 0.00 | 0.00 | 0.00 | 0.00 | 0.01 |
| Screening | 0.01 | 0.00 | 99.65 | 0.07 | 0.01 | 0.00 | 0.18 |
| Day of dosing | 0.26 | 5.65 | 67.88 | 0.99 | 0.01 | 0.21 | 20.90 |
| 1 week | 0.62 | 0.02 | 0.07 | 0.00 | 0.00 | 0.10 | 3.21 |
| 1 month | 0.72 | 0.05 | 1.25 | 0.00 | 0.00 | 0.00 | 2.33 |
| 2 months | 0.77 | 0.02 | 0.36 | 0.00 | 0.01 | 0.02 | 2.19 |
| 3 months | 1.01 | 0.01 | 0.73 | 0.01 | 0.02 | 0.02 | 2.42 |

### Example 7: Elevated Vaginal Inflammatory Markers in Dysbiotic Women

This Example demonstrates that dysbiotic vaginal microbiomes represent a local inflammatory state which is distinct from healthy women.

### Biobanking of Screening Cohort CVS for Biomarker Analysis

CVS samples were collected as described for the vaginal composition (see Example 2) but with the following differences: Samples were immediately placed on ice when received and kept cold throughout the entire processing procedure. Cold instead of room temperature sterile saline was used, and samples were frozen to −80° C. without the use of a CoolCell. Instead of adding 1 mL sterile saline per sample, 2.5 μL was added per mg sample so all samples were normalized for weight prior to analysis. The quality checks were omitted (CFU, pH, sperm cell check), except sequencing.

### Inflammatory Protein Analysis

Bacterial vaginosis (BV)-associated and dysbiotic vaginal microbiota have been linked to increased concentrations of several proinflammatory cytokines such as IL-1a, IL-1b, IL-8, IL-12, IL-18, and FMS-related tyrosine kinase 3 ligand (FLT-3L), as well as matrix metalloproteinases (MMPs), which in turn have been associated with increased rates of embryo implantation failure. The inflammatory status of the urogenital niche in turn affects the receptivity of the uterus and embryo implantation, and thereby IVF success.

### Proteomics (O-Link Analysis)

To investigate the inflammatory state of the asymptomatically dysbiotic and healthy women in the screening cohorts, 0-link analysis (BioXpedia, Aarhus, DK) was performed. O-link is a highly specific, targeted proteomics method, in which qPCR is performed on proteins by using antibody-linked oligonucleotides. Different panels of multi-well plates are available that target different subsets, such as the inflammation panel used here to quantify 92 inflammatory markers.

A clear separation based on inflammatory protein markers was observed between the dysbiotic and healthy groups in the screening cohort (FIG. 8A). Only one healthy microbiome sample clustered with the dysbiotic samples, which could be due to an undetermined infection that is not picked up by bacterial metagenome sequencing, such as, e.g., Candida (this data was generated pre-gynecological examination and pathogen screen, and all women were asymptomatic).

### Transcriptomics (NanoString)

To investigate the inflammatory state of the asymptomatically dysbiotic healthy women in the intervention cohort, NanoString nCounter technology (NanoString Technologies, Seattle, Washington) was applied to a subset of samples obtained from the healthy and dysbiotic subjects. NanoString uses a highly specific, targeted transcriptomics method in which individual mRNA molecules of an mRNA library are complexed with a uniquely barcoded complementary reporter probe before immobilization and detection via fluorescence. Different panels of reporter probes are available, such as the inflammation panel used here to quantify 579 inflammatory markers. A clear separation based on inflammatory mRNA markers was observed between the healthy donors and dysbiotic baseline recipients (FIG. 8B).

### Metabolomics (Untargeted)

To explore using metabolomics to investigate the difference between the asymptomatic dysbiotic, and healthy women in the intervention cohort, untargeted metabolomics (by MS-Omics Aps, Vedbæk, Denmark) using liquid chromatography/mass spectrometry was used to identify and characterize 879 small molecules in a subset of dysbiotic and healthy donor samples. A clear separation based on metabolic markers was observed between the healthy donors and dysbiotic baseline recipients (FIG. 8C).

The vaginal niche thus differs in healthy and asymptomatically dysbiotic women on RNA, protein and metabolite level.

Several proinflammatory cytokines were upregulated in the dysbiotic participants, such as IFNγ, IL-1α, IL-12, IL-18, and MMP-10 (FIG. 8D; Moscicki A-B, Shi B, Huang H, et al. (2020) Front. Cell. Infect. Microbiol. 10:569022.; Clara Aranha, Mayuri Goriwale, Shahina Begum, et al. (2022) Journal of Obstetrics and Gynaecology.; Jespers V, Kyongo J, Joseph S, et al. (2017) Scientific Reports 7: 11974.; Campisciano G, Zanotta N, Licastro D, et al. (2018) Scientific Reports 8:2307.; and Lannon S M R, Vanderhoeven J P, Eschenbach D A, et al. (2014) Reproductive Sciences 2014, Vol. 21(10) 1215-1227)), which belong to Th1 cytokines (IFNγ, IL-12), innate/inflammasome-associated cytokines (IL-1, IL-18), and metalloproteinases (e.g., MMP-10). Horizontal lines represent medians.

### Example 8: Administration of the Vaginal Composition Reduces the Inflammatory State of the Vaginal Niche

This Example describes the change of the local inflammatory state induced by administration of a vaginal composition by changing the dysbiotic vaginal microbiome.

### Biobanking of Donor and Recipient CVS for Biomarker Analysis

For several points in time, CVS samples taken from recipient 2 were used for Olink inflammatory biomarker analysis. Samples were processed in the same way as the biobanking samples of the screening cohort in Example 7. For each donor, the 5^{th} visit was not used for vaginal compositions but instead processed according to the biobanking method. This yielded a number of aliquots for the sole purpose of biobanking.

### Inflammatory Protein Analysis

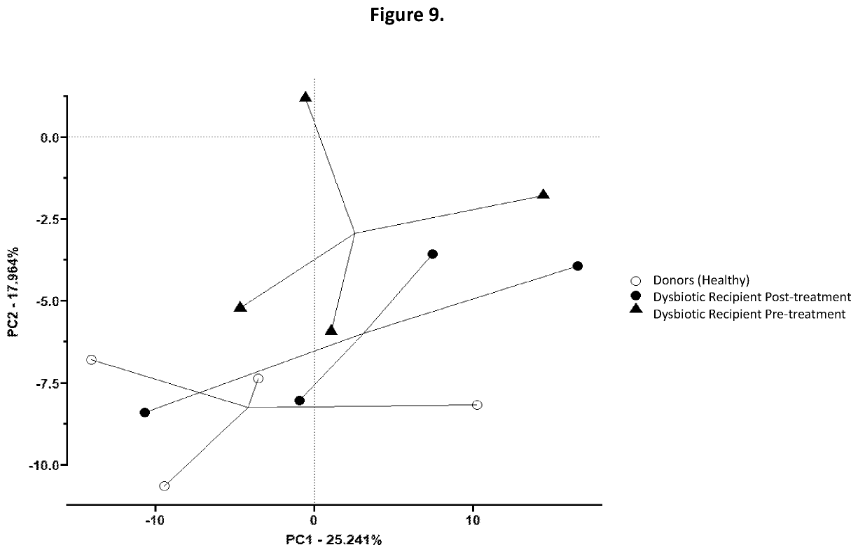

Prior to administration of the vaginal composition to the recipient, the samples of all dysbiotic subjects clustered together by 0-link proteomics, transcriptomics and metabolomics (FIGS. 8A, 8B and 8C). In FIGS. 8A, 8B, 8C each data point (e.g., square, circle, triangle) corresponds to the data of a healthy or dysbiotic subject, before receiving the treatment with the vaginal composition of the invention. In FIG. 9 each data point (solid circles, empty circles, solid triangles) corresponds to the data of a healthy or dysbiotic subject, or to a previously dysbiotic subject after receiving the treatment with the vaginal composition. The knot in each cluster represents the mean of a treatment group (FIGS. 8 and 9). The post-treatment analysis was performed between 1 week to 3 months after the administration of the vaginal composition to the dysbiotic subjects.

While the clusters and means of O-link proteomics data indicate that the dysbiotic (solid circles) and healthy donor (empty circles) vaginal microbiomes are distinct from each other, after administration of the vaginal composition, the cluster (solid triangles) shifted closer to the healthy donor cluster (empty circles in FIG. 9), which is indicative of a successful reversal of the vaginal dysbiosis. The administration of the vaginal composition thus appeared to have shifted the recipient's vaginal microbiome from a dysbiotic microbiome towards the microbiome of the healthy donor (FIG. 9).

Though limited by the small number of available data points, selected pro-inflammatory markers that were differentially expressed in dysbiotic and healthy cohorts (FIG. 8 D) were either not changed or showed a trend of being downregulated post-administration (data not shown), suggesting that the treatment of dysbiosis by administration of the vaginal composition may treat or ameliorate local inflammation in the vaginal cavity.

### Example 10: Multiparameter Assessment of Various Dosage Formulations Using Simulated Vaginal Fluid

A variety of suitable dosage forms was assessed for formulating the vaginal composition described herein, including formed gels, lyophilized gels, tablets, and films. A number of excipients were assessed to achieve suitable dosage forms, including mannitol, micro-crystalline cellulose, mucin (porcine, Sigma), hyaluronic acid (Sigma), maltodextrin, Guar gum (Sigma), inulin (Sigma), alginic acid (sodium alginate, Dupont), polyvinyl alcohol (PVA Parteck SRP 80, Merck), sodium CMC (Ac-Di-sol, sodium carboxymethyl cellulose, DuPont), polyvinylpyrrolidone (Kollidon (PVP), BASF), hydroxypropyl methylcellulose (Methocel K4M (HPMC), Colorcon), poloxamer (poloxamer 407 (Kolliphor), BASF), Carbopol (Carbopol 934, Serva), lactic acid, and acetate buffer.

### Basic Formulations

Formed gels were prepared using the excipients including, e.g., hyaluronic acid, sodium alginate, HPMC/PVP, and poloxamer 407. The gels were pH adjusted to maintain a pH about pH 3.4-pH 3.9. A combination of lactic acid and acetate buffers were assessed.

Tablets were prepared using the excipients such as bulking agents including, e.g., microcrystalline cellulose, HPMC/PVP, maltodextran, and poloxamer 407 and compression.

Lyophilized excipients were also evaluated to determine if mucoadhesion and/or gelling is improved. A combination of lactic acid and acetate buffer salts was assessed for inclusion. Tablet tensile strength was targeted at about 1.0 MPa, to ensure lack of excessive breakages and good processability. The target for pH of a reconstituted tablet (e.g., inside the vaginal cavity) was about pH 3.4-pH 3.9. The disintegration profile in a low liquid volume environment (e.g., inside the vaginal cavity) was also assessed.

For lyophilized gels, a number of excipients were lyophilized, including, e.g., hyaluronic acid, sodium alginate, HPMC/PVP, and poloxamer 407. All gels were pH adjusted to maintain a pH about pH 3.4-pH 3.9. A combination of lactic acid and acetate buffers were assessed. The target lyophilized appearance was a clean, uniform cake. The target water content was generally less than 3% w/w water (e.g., to increase viability of drug substance). The target reconstitution time in a vial was less than 2 minutes with hand swirling.

For films (air-dried) PVA in various concentrations was assessed. For example, PVA was supplemented with sodium CMC and other excipients to modify drying times, final flexibility of films, mucoadhesion etc. A combination of lactic acid and acetate buffers was assessed, and pH effect on immersion in simulated vaginal fluid evaluated. The target film properties included sufficient flexibility for application (e.g., to the vaginal tract), non-tackiness, acceptable drying times for ease of processing, a suitable film thickness, and disintegration profile in a low liquid volume environment (e.g., inside the vaginal cavity), as well as effective release and engrafting of drug substance. The target for pH of a reconstituted film (e.g., inside the vaginal cavity) was about pH 3.4-pH 3.9.

All components were assessed for optimisation of viscosity (e.g., at 37° C., e.g., inside the vaginal cavity), enhancement of mucoadhesion (e.g., to epithelial, mucosal surfaces e.g., inside the vaginal cavity) and/or positive impact on drug substance stability (e.g., bacterial viability upon formulation and storage). Ideal formulations show little to no flow on suitable vertical surfaces and maintain high bacterial viability (e.g., CFU count) both upon formulation and during (long-term) storage.

Formulation selection parameters that were assessed included: Mucoadhesion (of reconstituted product, e.g., in the vaginal tract); viscosity (of reconstituted product), e.g., final viscosity for gel-based product needs to be syringeable at ambient temperature and preferably congealed at 37° C. (at body temperature, e.g., in the vaginal tract); total sugar content (of reconstituted product), e.g., ideally at or lower than physiological concentration (about 0.5-1.0 mg/mL); volume of reconstituted product, e.g., up to 3 mL; hydration rate/disintegration rate (e.g., of gel/matrix), e.g., sufficient physical integrity to provide desired release rate; pH, e.g., between about pH 3.4-3.9 (e.g., to promote inhibition of competitive vaginal bacteria); water activity/moisture content (e.g., of dried formulations), e.g., between 0.5-3% water (e.g., for longer term dried formulation stability); microbial diversity, e.g., relative abundance of Lactobacillus species, such as, e.g., L. crispatus, L. gasseri, L. jensenii, and L. iners; total dose/potency, e.g., preferably above 1×10⁵ CFU/VCC, above 1×10⁶ CFU/VCC, above 1×10⁷ CFU/VCC, or above 1×10⁸ CFU/VCC per administration (per dose), shelf-life (not reconstituted) at various temperatures, and microbial limits, e.g., absence of microorganisms such as, Pseudomonas aeruginosa, Candida albicans, Staphylococcus aureus, Ph Eur criteria 5.1.4, 2.6.12 & 2.6.13).

Testing was performed using standard assays, including plate count (e.g., MRS agar) or viable cell count (VCC, e.g., using Quantom Tx automated counting system (AM620) with fluorescent stain), e.g., for life bacteria count, dose determination, shelf-life; rheometer, e.g., for mucoadhesion and viscosity, pH meter, Karl Fisher/water activity meter, Ph Eur testing, e.g., for microbial loads.

### Gel Formulations

Gel formulations were prepared with the following excipients:
Hyaluronic Acid at 0.5% and at 2%
HPMC+Carbopol+PVP
Poloxamer 20%+Sodium Alginate 2%
PVA 3%+NaCMC 2%
Poloxamer 20%+Guar gum 2%
Guar gum at 0.5% and 2%
Mucin 5%
Inulin 8%

Flow rates (viscosity, mPa×sec/shear rate 1/sec) under gravity at ambient temperature were determined with 0.5 mL on a 56 mm glass slide for Hyaluronic acid 0.5%, HPMC Kollidon 25+Carbopol, Guar gum 0.5%, Simulated mucus and inulin. Values obtained ranged from 300 to 1×10⁷ mPa×sec at shear rate of 0 to 3 to 7,000 mPa×sec at a shear rate of 100 1/sec, depending on formulation. Guar gum (0.5%) and hyaluronic acid (0.5%) did not appear to form gels, yielding low viscosities. Viscosity against temperature from 15 to 45° C. (with fixed shear rate of 10 s⁻¹) was also determined. Poloxamer showed a thermo-reversible gelling behaviour with an increase in viscosity at about 25° C. (from about 10,000 to about 12,000 mPa×sec, while other formulations mostly showed small changes in their viscosities at various levels between about 500 and 12,000 mPa×sec at 15° C. and about 200 to 11,000 at 45° C., depending on formulation.

The next step included and assessment which of the gel bases could be lyophilized and reconstituted to form an acceptable gel. The appearance of a ‘cake’ was also evaluated to indicate homogeneity.

A simulated vaginal fluid (SVF) was produced, with the following composition: 35 mg of NaCl, 14 mg of KOH, 22 mg of calcium hydroxide, 20 mg of lactic acid, 10 mg of acetic acid, 1.6 mg of glycerol, 50 mg of glucose into 10 mL of water, adjusting to pH 4.2 using HCL.

Single vials were used to simulate a single cervicovaginal secretion donation with SVF, and volumes used were considered as representative of the range of −0.5 mL to 1.5 mL expected from a typical donation. After suitable excipients were determined, these excipients were used to form gels by adding 1.5 mL of SVF. The addition of SVF allowed correct pH balance and simulate the addition of a vaginal composition to the excipient base. Samples were hand-shaken within vials to form homogenous gels and then lyophilized. Some excipients produced acceptable appearance post-lyophilisation. PVA, Guar gum and HA, formed acceptable (clear) gels; Kolliphor P 407 (Poloxamer 407) and Methocel K4M formed white, crystalline cakes; maltodextrin and PVP displayed discolorations (e.g., yellow), whereas sodium alginate, mucin, and NaCMC produced non-uniform cakes with discolorations (e.g., brown). Surprisingly, mannitol which typically forms consistently acceptable cakes, was crystalline which is probably due to the salt contents in the SVF.

The resulting gels were reconstituted with 1.5 mL deionised water post lyophilisation. HA, guar gum and PVA reconstituted easily, with reconstitution times of 2:30 min, 2:30 min, and 1:30 min, respectively. HA formed a homogenous gel with a pH of 4.6. Guar gum formed a non-homogenous gel with a pH of 4.5. PVA formed a homogenous liquid with some foaming with a pH of 4.7. PVP, NaCMC, poloxamer, sodium alginate and HPMC formed semi-homogenous gels. All required more than 3 minutes for reconstitution. PVP dissolved slowly with a pH of 4.1.

NaCMC formed a non-homogenous viscous liquid with a pH of 4.9. Poloxamer displayed some foaming with a pH of 5.4. HPMC formed a gel with some foaming with a pH of 4.4. Sodium alginate was non-homogenous with a pH of 5.5. Mannitol formed a homogenous liquid with some crystals remaining with a pH of 4.4.

A muco-adhesion test was conducted to study the effect under gravity of the drug product when applied. For this, a mucosal surface (such as would be present in the vaginal cavity) was simulated. 500 mg of mucin was compressed into disc a with a flat 2 cm tooling to approximately 3 tons of pressure. These were then adhered to a substrate, e.g., the bottom of a plastic box. Each disc was then wetted with approximately 200 μL of deionised water and rubbed with a gloved finger until the surface of the mucin became tacky to the touch. About 0.5 mL of sample (or the complete dosage unit, in the case of tablets and PVA films) was then applied in the horizontal position and allowed to settle for 2 minutes. The box was then raised so that samples were in a vertical position, and a visual assessment was taken with respect to adherence properties (muco-adhesion test) of the Guar gum, poloxamer, mannitol, PVP, hyaluronic acid, sodium alginate, CMC, HPMC, and PVA gel samples. Hyaluronic acid, NaCMC, sodium alginate and HPMC showed the best muco-adhesion in this test.

As poloxamer showed thermo-reversable behavior in earlier tests, the muco-adhesion testing was repeated on a pre-heated plastic box and mucin disc at 37° C. It was confirmed that the gel that formed was more viscous than at ambient temperature and retained on the surface of the wetted mucin disc. This indicates that the poloxamer may be at suitable viscosity at body-temperature Syringeability was also assessed following adjustment of concentrations: 3% (45 mg) carbopol, 4% (60 mg) sodium alginate, 3% (45 mg) HPMC & 1.9% (30 mg) sodium CMC, and 24% (360 mg) poloxamer. These were prepared using an SVF:lactate buffer mix. The inclusion of a lactate buffer in the pH range of −3.4-4 may promote engraftment of the lactic acid bacteria in vivo (e.g., in the vaginal tract), e.g., potentially by minimizing the competition of undesirable bacterial taxa resident in the existing microbial niche. Excipient were mixed with 1.5 mL SVF+lactate buffer. Carbopol and poloxamer both formed homogenous gels after hand shaking with some air bubbles which dispersed when settled. Sodium alginate and HPMC+Na-CMC did not form homogenous gels.

A lyophilized gel format as a dosage form for vaginal composition can, for example, be produced by blending with excipients followed by lyophilization and packaging, e.g., in vials.

The vials can then be reconstituted, e.g., in a clinic setting, with water to form a gel in the vial prior to application of the reconstituted drug product, e.g., by using an applicator, such as a syringe, to administer the vaginal composition to the vaginal tract of a subject.

A frozen gel describes a dosage form for the vaginal composition that is blended with gelling excipients (optionally along with a suitable lactate buffer) and the liquid gel form is then frozen (at −80° C.) and stored in either a vial or a pre-filled syringe, see, e.g., FIG. 13.

### Tablet Evaluation

In addition to gels, tablet-pessaries were generated as an additional dosage form. The following aspects were considered: choice of excipient suitable for compression to form tablets and for lyophilisation, as well as to provide acceptable level of mucoadhesion, optional inclusion of lactate buffer with a pH target of about pH 3.5-4, optionally with the aim to have a single tablet prepared from individual cervicovaginal fluid donations. All of the assessed excipients which produced both acceptable gel formation and acceptable lyophilisation (e.g., to form a reasonably free-flowing powder for further processing) were evaluated (alongside additional excipients) for tabletting quality on a manual tablet press, including Carbopol, HPMC, gelatine, sodium alginate, poloxamer, NaCMC, and pectin. Tablets were compressed at about 1 ton a with 5.5 mm tooling to form tablets. Tablets were gently added to wetted mucin discs and muco-adhesion assessed, as described earlier.

All tablets remained attached for over 5 minutes. Tablets were rinsed every minute to assess impact of adhesion. NaCMC began to swell into a gel. Tablets of carbopol, HPMC, alginate, NaCMC showed sufficient tablet integrity. Poloxamer formed a tablet but with relatively low strength. Pectin and gelatine tablets showed low hardness and some brittleness.

### Polymeric Films

Film forming/casting is a potential route of preserving viability in live biological materials. To investigate this, the following excipient test samples were generated:
PVA 3%
PVA 20%
PVA 3%, NaCMC 2%
PVA 20%, NaCMC 5%

All samples were mixed as necessary to make a gel/film and transfer into plastic blister pack (size 0) and were stored at ambient conditions for about 48 hours to set.

Both samples containing 3% PVA did not form suitable films. Films containing NaCMC displayed brittleness and were difficult to remove from the blister mould. The 20% PVA sample did form a film and could be removed with ease from the blister mould. The film was flexible and resistant to tearing.

To evaluate whether forming a film with the inclusion of vaginal composition is feasible, a ‘sandwich’ approach to film preparation was tried. This was performed in an upturned Karl Fischer lid (with PTFE lining), to produce a mould with a flat disc design. A 12% PVA solution (from the previous 20%) was used to reduce the initial viscosity and allow more consistent sample preparation via pipette. Preparation was performed as follows: 0.25 mL of 120 PVA was pipetted into each mould, followed by 0.5 mL of SVF (to simulate a vaginal composition). Another 0.25 mL of 12% PVA was pipetted on top to complete the ‘sandwich’. The moulds were left to solidify.

After about 48 hours, discs were formed, which were flexible and left no apparent residue on the PTFE lining. This suggests that the PTFE may be a suitable material for moulding, the film was easily recovered from this surface.

Films were gently added to mucin discs and muco-adhesion assessed, as described earlier. The PVA discs showed no movement from the mucin surface, indicating good muco-adhesion. A wash of about 2 mL of SVF was performed after 5 minutes, which did not displace the PVA discs from the mucin surface.

TABLE 5
Formulation summary:

| | | | Lyophilized / | |
|---|---|---|---|---|
| | | | frozen liquid | Film-form |
| Excipient | Functionality | Tablet | gel | (air-dried) |
| Hyaluronic acid | Structuring polymer | ++ | + | − |
| Sodium alginate | Structuring polymer | − | − | − |
| Maltodextran | Bulking agent, carbon | − | − | − |
| | source | | | |
| Guar gum | Bulking agent, prebiotic / | + | + | − |
| | viscosity agent | | | |
| NaCMC + HPMC | Polymer combination | + | + | − |
| Polyvinyl alcohol | Structuring polymer | − | − | ++ |
| Sodium CMC | Polymer / mucoadhsive | ++ | ++ | − |
| Polyvinylpyrrolidone | Structuring polymer | ++ | + | − |
| Hydroxypropyl | Structuring polymer | ++ | ++ | − |
| methylcellulose | | | | |
| (HPMC) | | | | |
| Poloxamer 407 | Structuring polymer | − | ++ | − |
| Carbopol 934 | Polymer / viscosity modifier | ++ | ++ | − |
| Mannitol | Control | − | − | − |
| Sodium lactate | pH modification | ++ | ++ | ++ |
| Lactic acid | pH modification / | ++ | ++ | ++ |
| | enhancement of drug | | | |
| | product effectiveness | | | |

++ suitable for dosage form
+ potentially suitable for dosage form
− not suitable for dosage form

### Example 11: Formulations of Dosage Forms Comprising a Vaginal Composition

Based on excipient data obtained from the formulation work using a simulated vaginal fluid (Example 10) and the viability testing performed (data not shown), a number of formulations were generated that contained vaginal compositions.

### Lyophilized Gel 1:

The formulation comprised NaCMC and a vaginal composition and was subjected to lyophilization.

30 mg of NaCMC was weighed and transferred into a lyophilization vial. 1 Vaginal composition was transferred into the lyophilization vial. 1 mL lactate buffer was used to rinse and take out any remaining vaginal composition from its vial and this was then transferred to the lyophilization vial. The vial was capped and swirled by hand to form a homogeneous gel that was then freeze-dried.

### Lyophilized Gel 2:

The formulation comprised poloxamer and a vaginal composition and was subjected to lyophilization.

240 mg of Poloxamer 407 was weighed and transferred into a lyophilization vial. 1 vaginal composition was transferred into the lyophilization vial. 1 mL lactate buffer was used to rinse and take out any remaining vaginal composition from its vial and this was then transferred to the lyophilization vial. The vial was capped and swirled by hand to form a homogeneous gel that is then freeze-dried.

### Frozen Gel 1:

The formulation comprised hyaluronic acid and a vaginal composition and was subjected to freezing at −80° C.

45 mg of hyaluronic acid was weighed and transferred into a lyophilization vial. 1 vaginal composition was transferred into the lyophilization vial. 1 mL lactate buffer was used to rinse and take out any remaining vaginal composition from its vial and this was then transferred to the lyophilization vial. The vial was capped and swirled by hand to form a homogeneous gel that was then frozen at −80° C.

### Frozen Gel 2:

The formulation comprised poloxamer and a vaginal composition and was subjected to freezing at −80° C.

240 mg of poloxamer 407 was weighed and transferred into a lyophilization vial. 1 vaginal composition was transferred into the lyophilization vial. 1 mL lactate buffer was used to rinse and take out any remaining vaginal composition from its vial and this was then transferred to the lyophilization vial. The vial was capped and swirled by hand to form a homogeneous gel that was then frozen at −80° C.

### Lyophilized Tablet 1:

The formulation comprised NaCMC and a vaginal composition and was subjected to lyophilization.

30 mg of NaCMC was weighed and transferred into a lyophilization vial. 1 vaginal composition was transferred into the lyophilization vial. 1 mL lactate buffer was used to rinse and take out any remaining vaginal composition from its vial and this was then transferred to the lyophilization vial. The vial was capped and swirled by hand to form a homogeneous gel that is then freeze dried. Once lyophilized, 300 mg of NaCMC were added to substance and compressed to 1 ton.

### Lyophilized Tablet 2:

The formulation comprised poloxamer and a vaginal composition and was subjected to lyophilization.

30 mg of Poloxamer 407 was weighed and transferred into a lyophilization vial. 1 vaginal composition was transferred into the lyophilization vial. 1 mL lactate buffer was used to rinse and take out any remaining vaginal composition from its vial and this was then transferred to the lyophilization vial. The vial was capped and swirled by hand to form a homogeneous gel that was then freeze-dried. Once lyophilized, 300 mg of PVP (Kollidon) were added to substance and compressed to 0.5 ton.

### PVA Film Disk

The formulation comprised 12% PVA and a vaginal composition sandwiched between two PVA layers (approximately 0.25 mL of PVA per layer).

Viable cell counts (VCC) were determined at t=0 after formulating the vaginal composition. Viability after 1 month and 2 months is also being determined and both VCC and CFU are assessed.

TABLE 6
Viable cell counts at t = 0 after formulation.

| | Donor #/ | CFU of | | Viable cell |
|---|---|---|---|---|
| Formulation | Visit # | donation | Species | count (VCC) |
| Lyophilized gel 1: CMC | E2 | 1.80E+08 | L. crispatus + L. iners | 5.94E+07 |
| | E6 | 8.70E+07 | L. crispatus + L. iners | 4.12E+07 |
| Lyophilized gel 2: | C4 | 4.60E+08 | L. crispatus + L. iners | 2.20E+08 |
| Poloxamer | C6 | 4.00E+08 | L. crispatus + L. iners | 1.14E+08 |
| Frozen gel 1: Hyaluronic | A1 | 7.30E+09 | L. crispatus | 7.86E+08 |
| acid | | | | |
| Frozen gel 2: Poloxamer | G8 | 1.70E+09 | L. jensenii + L. iners | |
| PVA disc | B3 | 5.10E+08 | L. crispatus | 1.95E+08 |
| Lyophilized tablet 1: CMC | D1 | 1.00E+08 | L. gasseri + L. iners | 6.28E+07 |
| | D7 | 2.20E+08 | L. gasseri + L. iners | 1.00E+07 |
| Lyophilized tablet 2: | D5 | 1.30E+08 | L. gasseri + L. iners | 4.60E+08 |
| Poloxamer/PVP | D9 | 1.30E+08 | L. gasseri + L. iners | 5.04E+08 |

Vaginal compositions can be lyophilized and formulated into, e.g., gels and tablets as well as other dosage forms that can be filled with lyophilized products, such as, e.g., capsules. Vaginal compositions can also be formulated into gels that can be frozen, as well as into liquid media (e.g., with glycerol) that can be frozen. Vaginal compositions can also be formulated into (air-dried) films, that could be, e.g., shaped like disks. Losses in viability range from approximately 0.5 log to 1 log at the formulation step depending on excipient and dosage form.

### Example 12: Successful Treatment of RPL Using Vaginal Composition

This example demonstrates the successful pregnancy resulting in live birth after administration of a vaginal composition to a subject having RPL and a dysbiotic vaginal microbiome.

### Patient History:

A 30-year-old woman with RPL (one successful pregnancy resulting in a liveborn boy (2017) followed by two stillbirths and one second trimester pregnancy loss). The first stillbirth in gestational week 27 (2019) was due to placental abruption. The autopsy was reported as normal.

The second pregnancy ended in gestational week 17 (2020) and was due to cervical insufficiency. Therefore, the patient received a prophylactic cervical cerclage in gestational week 13 of the next pregnancy. Unfortunately, that pregnancy ended due to a PPROM with no clinical sign of infection and resulted in a stillbirth at gestational week 23 (2020).

Following the first stillbirth, the patient was screened for antiphospholipid syndrome and thrombophilia with negative results.

During all prior pregnancies, the patient reported suffering from heavy vaginal discharge with yellow/green color, smelly odor, itching and pain (Table 7). She reported repeated treatments with antifungal medication followed by a recurrence in symptoms after a few weeks or no effect at all.

TABLE 7
Overview of symptoms.

| | | | | | | | Post administration | |
|---|---|---|---|---|---|---|---|---|
| | Pre administration | | Post administration | | | | April | May |
| | July | September | October | November | December | February | 2022 | 2022 |
| Date | 2021 | 2021 | 2021 | 2021 | 2021 | 2022 | GA: 5 + 2 | GA: 10 + 3 |
| Clinical observation (MD): | | | | | | | | |
| Colour (+/−) | + | + | − | − | − | − | − | − |
| Colour of vaginal fluid | Green/yellow | Yellow | White | White | White | White | White | White |
| | | | | | | lumps | | |
| Irritated vaginal m [symbol] | + | + | − | − | − | + | − | − |
| pH | | | | | | | | |
| Patient reported symptoms: | | | | | | | | |
| Itching/pain (+/−) | + | + | − | − | + | + | − | − |
| Odour (+/−) | + | + | − | − | − | + | − | − |
| Colour of vaginal fluid | Green/yellow | Green/yellow | White | White | Transparent | Yellow | White | White |
| Intensity of vaginal fluid | Heavy | Heavy | Light | Light | Light | Heavy | Light | Light |

| | Post administration | | | | | |
|---|---|---|---|---|---|---|
| | June | July | August | September | October | December |
| | 2022 | 2022 | 2022 | 2022 | 2022 | 2022 |
| Date | GA: [symbol] + 3 | GA: [symbol] | GA: [symbol] | GA: 25 + 2 | GA: 32 + 2 | GA: 37 + 5 |
| Clinical observation (MD): | | | | | | |
| Colour (+/−) | − | − | − | − | − | − |
| Colour of vaginal fluid | White | White | White | White | White | White |
| Irritated vaginal m [symbol] | − | − | − | − | − | − |
| pH | | | | | | |
| Patient reported symptoms: | | | | | | |
| Itching/pain (+/−) | − | + | − | − | − | − |
| Odour (+/−) | − | − | − | − | − | − |
| Colour of vaginal fluid | Transparent | White | White | White | White | White |
| Intensity of vaginal fluid | Light | Light | Light | Light | Light | Light |

GA: gestational age. [symbol]
indicates data missing or illegible when filed

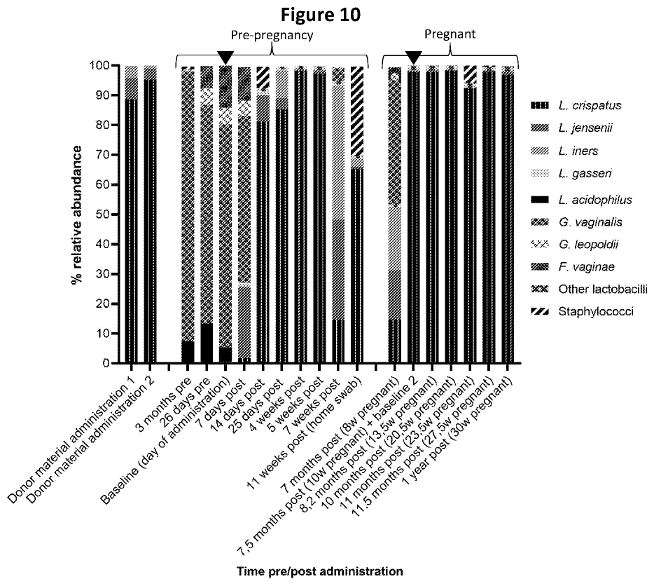

Despite negative results for APS and thrombophilia in 2019 after the first stillbirth, the patient was diagnosed with antiphospholipid syndrome (APS) based on her clinical history and increased values of beta-2-glycoprotein 1-IgG in august 2021. Due to persisting vaginal symptoms listed in Table 7, she was tested and confirmed to have vaginal dysbiosis (FIG. 10).

### Donor and Recipient Vaginal Microbiomes:

Vaginal microbiome sampling of the donor subject and recipient subject (RPL patient) was performed at the time points indicated in FIG. 10 using Zymo Collection Swab, 80XX (Zymo research, California US) were used and stored in COPAN (Brescia, Italy) containers. The sampling methodology was as indicated above in examples 1-5.

### Vaginal Microbiome Analysis/Extraction:

1 mL of each vaginal swab sample or 150 μL CVS was used for DNA extraction and microbiome analysis as in Example 1.

### Microbiome Sequencing and Bioinformatics:

DNA of donor and recipient vaginal samples was sequenced using the Illumina Miseq platform (Illumina, Inc., San Diego) producing 2×150 bp paired-end reads. The quality of the fastqc reads before and after quality trimming was assessed with FastQC v. 0.11.8 (20) Reads were quality trimmed using fastp v. 0.20.1 (21) with the parameters --qualified_quality_phred of 20 and minimum read length of 50. The reads were depleted of human sequences by aligning the trimmed reads to the human genome (hg38, University of California, Santa Cruz) using bowtie2 v. 2.3.4.1 (22) with end-to-end alignment and a maximum fragment length for valid paired-end alignments (−X) of 2000. Microbial profiling of the human DNA depleted reads was performed with Kraken 2 v. 2.1.2 (23) using the PlusPF database available from https://benlangmead.github.io/aws-indexes/k2 which includes RefSeq genomes of archaea, bacteria, viruses, plasmids, human, UniVec Core, protozoa, and fungi. Species and genus level abundances were estimated from the Kraken 2 reports using Bracken v. 2.6.2. The relative abundance of bacterial species and genera detected was calculated relative to the total number of reads classified as bacterial.

### In Vitro Assay for Donor Selection:

To select the best donor for the RPL patient, the ability of the bacteria in donor CVS to inhibit the growth of dysbiosis-associated bacteria in the patient was assessed using a plate diffusion assay. If the donor material inhibits growth of the bacteria in the patient CVS, a halo (inhibition zone) will form. The larger the halo around the well, the stronger the inhibition/exclusion. A CVS sample from the patient was processed according to the above-described protocol for donor CVS processing but without the quality controls. The sample was plated on G. vaginalis selective plates (ThermoFisher Scientific, PB5067A) and grown overnight (~18 hours) at 37° C. in an anaerobic jar with an Anaerogen anaerobic generator (ThermoFisher Scientific) to allow growth of the patient microbiota. After adding 1 mL sterile saline to the plate, colonies were scraped off, resuspended, and diluted to a density of about 0.5 McFarland standard. An amount of 100 μL of the diluted patient sample was plated on four new Gardnerella vaginalis plates and air-dried for 5 min. Next, four holes with a diameter of approx. 3 mm each were punched into each plate to create wells for insertion of donor material. Donor-derived CVS samples processed as described above were deposited into each well. There were 2 replicates with undiluted and 2 replicates with 5× diluted CVS for each donor. The plates were incubated anaerobically at 37° C. in an anaerobic jar with Anaerogen anaerobic generator. The presence and diameter of halos (inhibition zones) formed around the holes were measured after 2 and 5 days of incubation. The Gardnerella vaginalis medium used in the assay contained no antibiotics that are sensitive for lactobacilli for which Lactobacilli are sensitive.

### Administration of the Vaginal Composition:

The processed vaginal composition was stored in a cryovial in a −80° C. freezer. The thawing procedure consisted of: 1 hour at room temperature followed by 30 min in a 37° C. heating cabin. The vaginal composition was then drawn up into a 10 ml syringe on which a 180 mm Intrauterine insemination catheter (Wallace, 180 mm Catheter, Cooper Surgical, CT, USA) was mounted. The catheter was then introduced into the vagina (to the fornix) without the use of a speculum and the content of the syringe was applied. To ensure a complete emptying of the catheter, an additional 0.5 ml of air was drawn into the syringe and applied as well.

### Single Nucleotide Variant (SNV)-Analysis:

Samples at all timepoints where the coverage was high enough to generate SNV profiles both before and after the patient became pregnant, and before and after the two administrations of donor samples (i.e., two vaginal composition samples of the same donor subject), were used for analysis. L. crispatus SNV profiles were generated using MetaSNV under default settings, using a publicly available NCBI L. crispatus genome to generate the reference database. Reads were first aligned to the database using Bowtie2 and filtered to exclude alignments smaller than 45 base pairs and with less than 97% identity. Filtered reads were then used to generate the SNV profile of each metagenomic sample and Manhattan distances were calculated between profiles.

### Pre-Administration of Vaginal Composition:

The first patient sample showed a 90% dominance of G. vaginalis and a lack of Lactobacillus species (FIG. 10). At that time, the patient reported itching, odor, and heavy amounts of vaginal fluid that was green/yellow in color. On evaluation, irritation of the vaginal mucosa was noted, and the presence of odor and discoloration of the vaginal fluid was confirmed (Table 7). A sample taken shortly before the planned administration of the vaginal composition confirmed the microbial dysbiosis, including sustained predominance of G. vaginalis (FIG. 10) and the presence of symptoms associated with vaginal dysbiosis (Table 7).

### In Vitro Assay for Donor Selection:

In order to attempt to “match” a donor sample with the highest likelihood of successful engraftment in the patient, an in vitro competition assay was performed. The aliquots of cervicovaginal secretions (CVS) obtained from four thoroughly screened healthy donors with Lactobacillus-dominated microbiota were evaluated for inhibition of the vaginal dysbiosis (VD)-associated bacteria in the patient CVS using a plate diffusion assay. After 5 days, the undiluted samples from donors 1 and 2 both showed equally large inhibition zones or “halos” around the donor CVS (3 mm diameter), indicating similar inhibition of the VD-associated bacteria (Table 9, FIG. 10, 11). Subsequently, the donor CVS samples were then also diluted 5-fold prior to plating, which resulted in a slight reduction of the halo for donor 1 (1.5-2 mm) and a loss of the inhibitory effect for donor 2. The CVS from donors 3 and 4 were not effective in inhibiting microbial growth at either concentration. Based on these results, the vaginal composition obtained from donor 1 was selected for administration to the patient.

TABLE 8
Overview of donor sample properties and inhibition zones
(halos) in the in vitro assay for donor selection.

| | | | | | | | Halo | |
|---|---|---|---|---|---|---|---|---|
| | L. | L. | L. | L. | Other | | (mm) | Halo |
| Donor | crispatus | gasseri | iners | jensenii | lactobacilli | Selected vaginal | un- | (mm) |
| ID | % | % | % | % | % | pathogens % | diluted | 5x diluted |
| 1 | 88.7 | <0.05 | 4.0 | 7.2 | <0.05 | 0 | 3 | 1.5-2 |
| 2 | 97.6 | 0 | 0.3 | 2.0 | 0.1 | 0 | 3 | 0 |
| 3 | 97.3 | 2.2 | 0 | 0.1 | 0.2 | 0 | 0 | 0 |
| 4 | 0 | 0 | 99.6 | 0 | 0 | 0.1 | 0 | 0 |

The percentages for each species are relative abundances as determined by whole metagenome shotgun sequencing of the donor sample used in the assay. The pH and number of viable cells of the donor samples used in the assay were between 3.8-4.1 and 3.1E08-1.0E11, respectively. The sample of donor 1 was selected for administration to the recipient.

### Administration of the Vaginal Composition to the Patient:

An administration of the donor's vaginal composition to the vaginal cavity of the recipient subject, was performed without antibiotic pretreatment on menstrual cycle day 10. On the day of the administration, the vaginal microbiome was confirmed to be dysbiotic with 80% G. vaginalis and 14% F. vaginae and the pH was 4.25 (FIG. 10). The composition of the donor vaginal composition samples (donor 1 in Table 8) used for the administration to the recipient subject was 88.7% L. crispatus, 7.2% L. jensenii and 4% L. iners with a pH of 4.1 (Table 8). At 14 days post administration, the patient's microbiome had shifted to 81.2% L. crispatus and 9% L. jensenii and the pH was 3.87 (FIG. 10). The patient remained Lactobacillus dominant, mainly driven by L. crispatus at 6 sampling times during a 4-month follow-up period, except for one time, post-administration where L. crispatus was mixed with L. iners and L. jensenii that were also present in the donor vaginal composition sample (FIG. 10).

The patient experienced a significant improvement of VD-symptoms with a reduction in amount of vaginal discharge, change of color of the CVS to transparent/light white, and disappearance of symptoms such as itching and pain only 2 days post administration and throughout the following 4 months after administration of the vaginal composition of the donor (Table 7).

Pregnancy Post Treatment with Donor's Vaginal Composition:

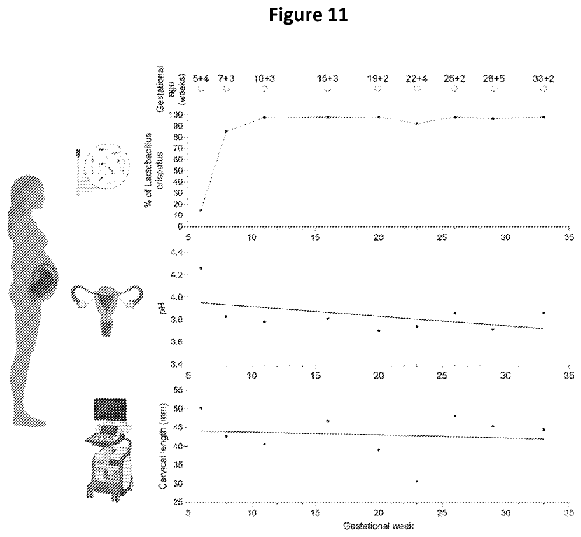

After administration of the vaginal composition, and reverting the dysbiotic microbiome, the patient became pregnant. It is under debate whether acetylsalicylic-acid and heparin have any beneficial effect on RPL, but due to the low risk, patient was treated with daily 75 mg acetylsalicylic-acid from the time of a positive pregnancy test until gestational week 38+0, and low-molecular-weight heparin 4500 Units daily from the time of a positive pregnancy test until six weeks postpartum. Throughout the subject's pregnancy, she was examined at a hospital every 3-4 weeks. A vaginal ultrasound to measure cervical length, a gynecological examination, a vaginal swab for microbiome analysis, sample collection for pH measurements, and an interview regarding vaginal symptoms were performed during these visits (Table 7 and FIG. 11).

FIG. 10 shows the stable L. crispatus dominated vaginal microbiome throughout her pregnancy. At one timepoint post-administration, at gestational age 5+2, the microbiome analysis revealed 41.7% of G. vaginalis. Subsequently, a second administration with a second sample from the same donor was performed. However, the sample taken directly before this second administration showed that the recipient had reverted again to the donor microbiome prior to the second administration and hence the first administration is thought to have recovered from the temporary reversal to dysbiosis without the need for a second intervention.

The compositions of both vaginal composition samples are shown in Table 9.

TABLE 9
Sample properties and quality control results for the two samples
obtained from the donor used for administration to the recipient.

| | | | | | | Selected | | | | | Viable |
|---|---|---|---|---|---|---|---|---|---|---|---|
| | | | | | | vaginal | | | | Viable | cells/ |
| | L. | L. | L. | L. | Other | dysbiosis- | | | | cells/ | dose - |
| | [symbol] rispatus | [symbol] asseri | [symbol] ners | [symbol] ensenii | lactobacilli | associated | | Weight | Dose | dose - | after |
| Sample ID | % | % | % | % | % | bacteria % | pH | (mg) | (mL) | fresh | storage |
| Sample 1 | 88.7 | <0.05 | 4.0 | 7.2 | <0.05 | 0 | 4.1 | 453 | 1.6 | 3.1E08 | 9.7E08 |
| Sample 2 | 95.3 | 0 | 1.0 | 3.7 | 0.1 | 0 | 3.9 | 1035 | 1.8 | 6.5E07 | 1.6E07 |

[symbol]

indicates data missing or illegible when filed

### The Percentages for Each Species are Relative Abundances as Determined by Whole Metagenome Shotgun Sequencing of the Donor Sample Used in the Assay

The cervical length remained stable and above 3 cm throughout pregnancy and vaginal pH measurements were all well below 4.5 in all samples (FIG. 11). In contrast to the subject's previous pregnancies, she did not suffer from VD-symptoms throughout this pregnancy.

The subject underwent a planned, uncomplicated cesarean section at gestational age 37+5 resulting in a live birth.

### Confirmation of Donor Engraftment Via SNV Analysis:

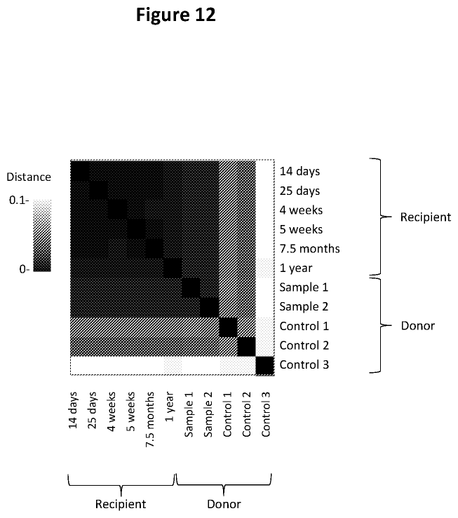

To confirm the engraftment of the donor L. crispatus strain(s), a SNV-analysis was performed to compare the genomic profiles of the L. crispatus sequenced in the vaginal compositions of the samples used for administration to the patient, CVS obtained from other donors that was not used for administration (Control 1-3), and in the patient after administration. The L. crispatus SNV profiles of the patient after administration of the vaginal composition were consistently similar to the two vaginal composition samples used for administration (Donor 1A and Donor 1B) (FIGS. 12 and 13). The SNV profiles of the donor samples were also very similar to each other, reflecting the stable presence of the L. crispatus used for administration to the patient. Moreover, when compared to the L. crispatus genomes in the vaginal composition samples from other donors that were not used to treat the patient, the L. crispatus in the patient samples after administration were more similar to the vaginal composition donor than to other healthy controls. L. crispatus observed in the recipient after the administration of the vaginal composition comprises the same L. crispatus variant that was present in the donor vaginal composition samples and remained the dominant variant throughout pregnancy for up to 1 year after the first administration (FIG. 10, FIG. 12). This confirms at the strain level stable engraftment of the donor's vaginal microbiome in the recipient

The administration of a vaginal composition reverted the dysbiotic microbiome of a subject having RPL, resulting in a successful pregnancy with live birth without the need for antibiotic pre-treatment.

### REFERENCES

Anahtar M N, Byrne E H, Doherty K E, Bowman B A, Yamamoto H S, Soumillon M, Padavattan N, Ismail N, Moodley A, Sabatini M E, Ghebremichael M S, Nusbaum C, Huttenhower C, Virgin H W, Ndung'u T, Dong K L, Walker B D, Fichorova R N, Kwon D S. 2015. Cervicovaginal bacteria are a major modulator of host inflammatory responses in the genital tract. Immunity 42:965-976. https://doi.org/10.1016/j.immuni.2015.04.019
Lennard K, Dabee S, Barnabas S L, Havyarimana E, Blakney A, Jaumdally S Z, Botha G, Mkhize N N, Bekker L-G, Lewis D A, Gray G, Mulder N, Passmore J-A S, Jaspan H B. 2018. Microbial composition predicts genital tract inflammation and persistent bacterial vaginosis in South African adolescent s. Infect Immun 86:e00410-17. https://doi.org/10.1128/IAI.00410-17.
Masson L, Mlisana K, Little F, Werner L, Mkhize N N, Ronacher K, Gamieldien H, Williamson C, Mckinnon L R, Walzl G, Abdool Karim Q, Abdool Karim S S, Passmore J-A S. 2014. Defining genital tract cytokine signatures of sexually transmitted infections and bacterial vaginosis in women at high risk of HIV infection: a cross-sectional study. Sex Transm Infect 90:580-588. https://doi.org/10.1136/sextrans-2014-051601
Fichorova R N, Morrison C S, Chen P-L, Yamamoto H S, Govender Y, Junaid D, Ryan S, Kwok C, Chipato T, Salata R A, Doncel G F. 2020. Aberrant cervical innate immunity predicts onset of dysbiosis and sexually transmitted infections in women of reproductive age. PLoS One 15:e0224359. https://doi.org/10.1371/journal.pone.0224359.
Garrett N., Mtshali, A., Osman, F. et al. Impact of point-of-care testing and treatment of sexually transmitted infections and bacterial vaginosis on genital tract inflammatory cytokines in a cohort of young South African women. Sex Transm Infect. 2021 December; 97(8):555-565.
Camargo Campos A C, Candido Murta E F, Michelin M A, and Reis C. Evaluation of Cytokines in Endocervical Secretion and Vaginal pH from Women with Bacterial Vaginosis or Human Papillomavirus. Obstetrics and Gynecology Volume 2012, Article ID 342075.
Cherne M D, Cole A L, Newberry L, Schmidt-Owens M, Deichen M, Cole A M. Matrix Metalloproteinases Expressed in Response to Bacterial Vaginosis Disrupt the Endocervical Epithelium, Increasing Transmigration of HIV. Infect Immun. 2020 Mar. 23; 88(4):e00041-20.
Haahr T. et al., Abnormal vaginal microbiota may be associated with poor reproductive outcomes: a prospective study in IVF patients. Hum Reprod. 2016 April; 31(4):795-803. doi: 10.1093/humrep/dew026. Epub 2016 Feb. 23. PMID: 26911864.
Koedooder R. et al., The vaginal microbiome as a predictor for outcome of in vitro fertilization with or without intracytoplasmic sperm injection: a prospective study. Hum Reprod. 2019 Jun. 4; 34(6):1042-1054. doi: 10.1093/humrep/dez065. Erratum in: Hum Reprod. 2019 Oct. 2; 34(10):2091-2092. PMID: 31119299.
Moreno I. et al., Endometrial microbiota composition is associated with reproductive outcome in infertile patients. Microbiome. 2022 Jan. 4; 10(1):1. doi: 10.1186/s40168-021-01184-w. PMID: 34980280; PMCID: PMC8725275.
Wiesenfeld H C et al., Lower genital tract infection and endometritis: insight into subclinical pelvic inflammatory disease. Obstet Gynecol. 2002 September; 100(3):456-63. doi: 10.1016/s0029-7844(02)02118-x. PMID: 12220764.
Haggerty C L et al., PID Evaluation and Clinical Health study investigators. Bacterial vaginosis and anaerobic bacteria are associated with endometritis. Clin Infect Dis. 2004 Oct. 1; 39(7):990-5. doi: 10.1086/423963. Epub 2004 Sep. 2. PMID: 15472851.
Campisciano G, Florian F, D'Eustacchio A, Stanković D, Ricci G, De Seta F, Comar M. Subclinical alteration of the cervical-vaginal microbiome in women with idiopathic RPL. J Cell Physiol. 2017 July; 232(7):1681-1688. doi: 10.1002/jcp.25806. Epub 2017 Feb. 16. PMID: 28098358.
Spandorfer S D et al., Relationship of abnormal vaginal flora, proinflammatory cytokines and idiopathic RPL in women undergoing IVF. J Reprod Med. 2001 September; 46(9):806-10. PMID: 11584481.
Zhao C et al., Characterization of the Vaginal Microbiome in Women with RPL and Its Potential Correlation with Hormone Stimulation during In Vitro Fertilization Surgery. mSystems. 2020 Jul. 14; 5(4):e00450-20. doi: 10.1128/mSystems.00450-20. PMID: 32665329; PMCID: PMC7363005.
Wilson J D et al., Rates of bacterial vaginosis in women undergoing in vitro fertilisation for different types of RPL. BJOG. 2002 June; 109(6):714-7. doi: 10.1111/j.1471-0528.2002.01297.x. PMID: 12118653.
Jiang I et al., Intricate Connections between the Microbiota and Endometriosis. Int J Mol Sci. 2021 May 26; 22(11):5644. doi: 10.3390/ijms22115644. PMID: 34073257; PMCID: PMC8198999.
Wei W et al., Microbiota composition and distribution along the reproductive tract of women with endometriosis. Ann Clin Microbiol Antimicrob. 2020 Apr. 16; 19(1):15. doi: 10.1186/s12941-020-00356-0. PMID: 32299442; PMCID: PMC7161132.
Salah et al. Bacterial vaginosis and RPL: Cause or association?Eur J Obstet Gynecol Reprod Biol. 2013 March; 167(1):59-63. doi: 10.1016/j.ejogrb.2012.10.031. PMID: 23199811 Hemmerling et al. Phase 2a study assessing colonization efficiency, safety, and acceptability of lactobacillus crispatus CTV-05 in women with bacterial vaginosis. Sex Transm Dis. 2010 December; 37(12):745-50. doi: 10.1097/OLQ.0b013e3181e50026. PMID: 20644497
Ngugi et al. Effects of bacterial vaginosis-associated bacteria and sexual intercourse on vaginal colonization with the probiotic Lactobacillus crispatus CTV-05. Sex Transm Dis. 2011 November; 38(11):1020-7. doi: 10.1097/OLQ.0b013e3182267ac4. PMID: 21992977 PMCID: PMC3193282
Gajer et al. Temporal dynamics of the human vaginal microbiota. Sci Transl Med. 2012 May 2; 4(132):132ra52. doi: 10.1126/scitranslmed.3003605. PMID: 22553250 PMCID: PMC3722878
Ravel et al. Vaginal microbiome of reproductive-age women. Proc Natl Acad Sci USA. 2011 Mar. 15; 108 Suppl 1(Suppl 1):4680-7. doi: 10.1073/pnas.1002611107. PMID: 20534435 PMCID: PMC3063603

1. A method of treating recurrent pregnancy loss (RPL), comprising administering a therapeutically effective amount of a vaginal composition to a genitourinary tract of a subject in need thereof, wherein the subject exhibits a dysbiotic microbiota in the genitourinary tract, further wherein the vaginal composition comprises:
(i) one, two, three or four bacterial species from the genus Lactobacillus which comprise about 80% to about 99.9% of all detectable bacterial species of the vaginal composition, optionally, wherein the bacterial species are selected from Lactobacillus crispatus, Lactobacillus iners, Lactobacillus jensenii, Lactobacillus gasseri;
(ii) less than 5% of Gardnerella spp., Atopobium spp., and Prevotella spp.; and
(iii) vaginal transudate or mucus.

2. The method of claim 1, wherein the vaginal composition is administered to the vagina of the subject.

3. (canceled)

4. The method of claim 1, wherein the dysbiotic microbiota in the genitourinary tract is reverted to a Lactobacillus-dominated microbiota, wherein the Lactobacillus-dominated microbiota comprises at least 60% Lactobacillus species.

5-7. (canceled)

8. The method of claim 1, wherein the subject is not pregnant at the administration of a first dose of the vaginal composition, and is pregnant at the administration of a second, third, or fourth dose of the vaginal composition.

9. The method of claim 1, wherein the administration of the vaginal composition is maintained for at least 1, and wherein the administration of the vaginal composition comprises up to 4 or more doses.

10. The method of claim 1, wherein the vaginal composition is administered at regular intervals, wherein the regular intervals comprise weekly, biweekly, or monthly.

11-15. (canceled)

16. The method of claim 1, wherein the vaginal composition is administered as part of a combination therapy, wherein the vaginal composition is administered in combination with a second therapeutic agent or assisted reproductive treatment.

17. The method of claim 1, wherein the second therapeutic agent comprises an anticoagulant, acetyl-salicylic acid, heparin, progesterone, thyroid hormone replacement, metformin, insulin, or an antibiotic agent.

18-21. (canceled)

22. The method of claim 16, wherein the second therapeutic agent is administered at the same time as the vaginal composition.

23. The method of claim 16, wherein the second therapeutic agent is administered at a different time point than the vaginal composition.

24. (canceled)

25. The method of claim 1, wherein the use of the vaginal composition increases the rate of full-term births in a population of subjects having dysbiotic microbiota in the genitourinary tract and RPL, relative to a control population.

26. The method of claim 1, wherein the vaginal composition is formulated into a dosage form, wherein the dosage form comprises an effective amount of the vaginal composition in one or more discrete units, wherein the effective amount is predetermined.

27-29. (canceled)

30. The method of claim 1, wherein a vaginal wash is performed with saline, lactic acid or an antiseptic agent.

31. The method of claim 1, wherein:
(a) the subject has been diagnosed with bacterial vaginosis or a vaginal infection, and is pre-treated with an antibiotic prior to administration of the vaginal composition; or
(b) the subject has not been diagnosed with bacterial vaginosis or a vaginal infection, and is not pre-treated with an antibiotic prior to administration of the vaginal composition.

32-37. (canceled)

38. The method of claim 1, wherein after administration of the vaginal composition to the subject:
(a) elevated vaginal levels of pro-inflammatory or inflammasome-associated cytokines in the subject are reduced;
(b) elevated vaginal levels of Th1 cytokines in the subject are reduced; or
(c) reduced vaginal levels of at least IGF-1, IL-4, IL-10, TGFβ or CCL5 in the subject are increased.

39. (canceled)

40. The method of claim 1, wherein the vaginal composition further comprises a pharmaceutically acceptable carrier or diluent.

41-44. (canceled)

45. The method of claim 9, wherein the doses are administered if the genitourinary tract microbiota is determined to be dysbiotic, wherein dysbiotic, comprises a predetermined level of pathogens or pathobionts.

46-47. (canceled)

48. The method of claim 45, wherein a dose of the vaginal composition is at least 1×10³ colony forming units (CFUs).

49-54. (canceled)

55. The method of claim 1, wherein about 80% to about 99.9% of all detectable bacterial species of the vaginal composition consist of:
(a) Lactobacillus crispatus;
(b) Lactobacillus iners;
(c) Lactobacillus crispatus and Lactobacillus iners;
(d) Lactobacillus crispatus and Lactobacillus jensenii;
(e) Lactobacillus crispatus, Lactobacillus iners and Lactobacillus jensenii;
(f) Lactobacillus crispatus, Lactobacillus gasseri and Lactobacillus jensenii;
(g) Lactobacillus iners and Lactobacillus jensenii;
(h) Lactobacillus iners, Lactobacillus jensenii, and Lactobacillus gasseri;
(i) Lactobacillus iners and Lactobacillus gasseri; or
(j) Lactobacillus crispatus, Lactobacillus iners, Lactobacillus jensenii, and Lactobacillus gasseri.

56. (canceled)

57. The method of claim 1, wherein less than 10% of all detectable bacterial species of the dysbiotic microbiota in the genitourinary tract of the subject belong to the genus Lactobacillus, and at least 20% or more of all detectable bacterial species of the dysbiotic microbiota are pathogens or pathobionts comprising Gardnerella spp., Atopobium spp., or Prevotella spp.

58. The method of claim 1, wherein the vaginal composition is in the form of a suspension, spray, gel, cream, ointment, semi-solid foam, film, powder, capsule, solution for lavages or douches, ovules, a vaginal insert, tampon, tablets or a microencapsulated product.

59-61. (canceled)

62. A method of producing a vaginal composition, wherein the method comprises the steps of:
(a) processing a microbiota sample from a donor female genitourinary tract, wherein the microbiota sample comprises a vaginal mucosal sample or cervicovaginal secretion, further comprising urogenital microbes and vaginal mucosal fluid from the vaginal cavity of a healthy donor subject;
(b) assessing the absence of one or more pathogens;
(c) assessing viability or quantity of the urogenital microbes, and
(d) releasing the vaginal composition comprising the microbiota sample from quarantine for use in an assisted reproductive procedure, if a predetermined level is obtained in step (b) and (c).

63-65. (canceled)
